(19) United States
(12) Patent Application Publication
(10) Pub. No.: US 2016/0199399 A1
(43) Pub. Date: Jul. 14, 2016

(54) METHODS FOR PREDICTING DRUG RESPONSIVENESS IN CANCER PATIENTS

(71) Applicant: Medical Prognosis Institute A/S, Horsholm (DK)

(72) Inventor: Steen Knudsen, Scottsdale, AZ (US)

(21) Appl. No.: 14/991,080

(22) Filed: Jan. 8, 2016

Related U.S. Application Data
(60) Provisional application No. 62/101,618, filed on Jan. 9, 2015.

(51) Int. Cl. A61K 31/7048; A61K 31/664; A61N 5/10; A61K 31/704; C12Q 1/68; A61K 45/06; A61K 31/4745

(52) U.S. Cl. CPC A61K 31/7048; A61K 45/06; A61K 31/664; A61K 31/4745; A61K 31/704; C12Q 1/6886; A61N 5/10; C12Q 2600/178; C12Q 2600/158; C12Q 2600/106

(57) ABSTRACT

The invention features methods for predicting responsiveness to chemotherapy drug(s) in cancer patients (e.g., patients with lymphoma (e.g, patients with DLBCL)), by, e.g., determining a level of expression of one or more biomarkers in a biological sample obtained from the patient.

### FIELD OF THE INVENTION

The invention features methods to predict responsiveness to chemotherapy drugs in cancer patients.

### BACKGROUND

Diffuse large B-cell lymphoma (DLBCL) is the most common B-cell malignancy and accounts for 40% of all adult non-Hodgkin type lymphomas. DLBCL is highly heterogenous in both clinical and molecular aspects. The current standard for DLBCL treatment includes the chemotherapy drug combination of cyclophosphamide, doxorubicin, vincristine, and prednisolone or prednisone (known as CHOP), which is often administered in combination with the anti-CD20 monoclonal antibody Rituximab (R-CHOP). Treatment with the combination therapy of R-CHOP has been shown to cure more than 50% of patients with DLBCL and to increase 5 year survival rates by more than 60% (Akyurek et al, Cancer. 118(17): 4173-83 (2011); Feugier et al, J. Clin. Oncol. 23(18): 4117-4126, 2005)). Other treatments administered for DLBCL include the combination of chemotherapy drugs known as DHAP (dexamethasone, ara-c, and cisplatin) and ICE (ifosfamide, carboplatin, and etoposide). However, a significant number of patients will relapse after or will remain refractory to these chemotherapy treatments.

Gene expression analysis can reveal the presence of a disease, such as cancer, in a patient, its type, stage, and origin, and whether genetic mutations are involved. Gene expression analysis can also be used to predict the efficacy of therapies. For example, the National Cancer Institute (NCI) has tested compounds for their effect in limiting the growth of 60 human cancer cell lines. NCI has also measured gene expression in those 60 cancer cell lines using DNA microarrays. Various studies have explored the relationship between gene expression and compound effect using the NCI datasets.

Identification of molecular biomarkers that can be used in clinical settings to predict patient responsiveness to specific treatment regimens would allow for better determinations of which therapies will be most effective for an individual patient and for optimal treatment of patients that suffer a relapse. Thus, there exists a need in the art for methods that can be used to predict the responsiveness of cancer patients, such as DLBCL patients, to treatment with chemotherapeutic drugs.

### SUMMARY OF THE INVENTION

The invention features methods for predicting the responsiveness of a patient (e.g., a cancer patient, such as a lymphoma patient (e.g., a patient with DLBCL)) to a treatment for cancer, e.g., by determining the level of expression of one or more biomarkers (e.g., one, two, three, four, five, ten, twenty, or all biomarkers for each respective therapy shown in Table 1). Exemplary treatments for cancer include, e.g., an anti-cancer agent (e.g., a chemotherapy drug), such as cyclophosphamide, vincristine, and etoposide (COPE); methotrexate, carmustine, and teniposide (MBVP); dexamethasone, ara-c, and cisplatin (DHAP); ifosfamide, carboplatin, and etoposide (ICE); etoposide, ifosfamide, and methotrexate (VIM); methyl-GAG, ifosfamide, methotrexate, and etoposide (MIME); etoposide, cytarabine, and cisplatin (ESHAP); cyclophosphamide, carmustine, and etoposide (CBV); cyclophosphamide, etoposide, vincristine, and doxorubicine (EPOCH); fludarabine, cyclophosphamide, and mitoxantrone (FCM); cyclophosphamide, vincristine, doxorubicin, and dexamethasone (CVAD-A); methotrexate and cytarabine (CVAD-B); ifosfamide, mitoxantrone, and etoposide (MINE); mechlorethamine, vincristine, and procarbazine (MOPP); cyclophosphamide, vincristine, adriamycine, and etoposide (CHOEP); cyclophosphamide, vincristine, adriamycine, and belinostat (BeICHOP); methotrexate (MTX); doxorubicin; bendamustine; adriamycin, bleomycin, vinblastine, and dacarbazine (ABVD); cyclophosphamide, vincristine, adriamycine (CHOP); prednisolone; and gemcitabine and oxaliplatin (GemOx).

A first aspect of the invention features a method of predicting the responsiveness to treatment in a patient (e.g., a cancer patient, such as a lymphoma patient (e.g., a patient with DLBCL)) by determining a level of expression of a first biomarker (e.g., hsa-miR-766_st and/or hsa-miR-34b_st) in a biological sample (e.g., a tumor biopsy) from the patient. The treatment may be selected from, e.g., COPE, MBVP, DHAP, ICE, VIM, MIME, ESHAP, CBV, EPOCH, FCM, CVAD-A, CVAD-B, MINE, MOPP, CHOEP, BeICHOP, methotrexate (MTX), doxorubicin, and/or bendamustine. In particular, a change (e.g., an increase or a decrease) in the level of expression of hsa-miR-766_st and/or hsa-miR-34b_st can be used to indicate whether the patient will be responsive to the treatment.

In an embodiment, the method may also include determining the level of expression of one or more second biomarkers (e.g., one, two, three, four, five, ten, twenty, or all biomarkers for each respective therapy shown in Table 1) in the biological sample (e.g., a tumor biopsy) from the patient (e.g., a cancer patient, such as a lymphoma patient (e.g., a patient with DLBCL)) or in a second biological sample from the patient. In particular, a change (e.g., an increase or decrease) in the level of expression of the one or more of the second biomarkers indicates that the patient will be responsive to the treatment (e.g., treatment with COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, and/or bendamustine).

For example, when the treatment is COPE, the second biomarkers may be selected from any one or more (e.g., one, two, three, four, five, ten, twenty, or all biomarkers) of ACA48_x_st, ENSG00000202498_x_st, HBII-85-26_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and/or hsa-miR-93-star_st.

For example, when the treatment is MBVP, the second biomarkers may be selected from any one or more (e.g., one, two, three, four, five, ten, twenty, or all biomarkers) of ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and/or hsa-miR-93-star_st.

For example, when the treatment is DHAP, the second biomarkers may be selected from any one or more (e.g., one, two, three, four, five, ten, twenty, or all biomarkers) of ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2_s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and/or hsa-miR-938_st.

For example, when the treatment is ICE, the second biomarkers may be selected from any one or more (e.g., one, two, three, four, five, ten, twenty, or all biomarkers) of 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and/or hsa-miR-938_st.

For example, when the treatment is VIM, the second biomarkers may be selected any one or more (e.g., one, two, three, four, five, ten, twenty, or all biomarkers) of from ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, when the treatment is MIME, the second biomarkers may be selected from any one or more (e.g., one, two, three, four, five, ten, twenty, or all biomarkers) of ACA10_s_st, ACA11_st, ACA13_st, ACA18_x_st, ACA21_st, ACA40_x_st, ACA48_x_st, ACA51_x_st, ACA55_st, ACA61_st, ACA7_s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, when the treatment is ESHAP, the second biomarkers may be selected from any one or more (e.g., one, two, three, four, five, ten, twenty, or all biomarkers) of ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and/or hsa-miR-768-5p_st.

For example, when the treatment is CBV, the second biomarkers may be selected from any one or more (e.g., one, two, three, four, five, ten, twenty, or all biomarkers) of ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and/or hsa-miR-938_st.

For example, when the treatment is EPOCH, the second biomarkers may be selected from any one or more (e.g., one, two, three, four, five, ten, twenty, or all biomarkers) is selected from any one or more of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st.

For example, when the treatment is FCM, the second biomarkers may be selected from any one or more (e.g., one, two, three, four, five, ten, twenty, or all biomarkers) of ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and/or hsa-miR-92b_st.

For example, when the treatment is CVAD-A, the second biomarkers may be selected from any one or more (e.g., one, two, three, four, five, ten, twenty, or all biomarkers) of ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and/or hsa-miR-92a-2-star_st.

For example, when the treatment is CVAD-B, the second biomarkers may be selected from any one or more (e.g., one, two, three, four, five, ten, twenty, or all biomarkers) of ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_st, hsa-miR-663b_st, hsa-miR-768-5p_st, and/or hsa-miR-92a_st.

For example, when the treatment is MINE, the second biomarkers is selected from any one or more (e.g., one, two, three, four, five, ten, twenty, or all biomarkers) of 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and/or hsa-miR-938_st.

For example, when the treatment is MOPP, the second biomarkers may be selected from any one or more (e.g., one, two, three, four, five, ten, twenty, or all biomarkers) of ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and/or hsa-miR-92b_st.

For example, when the treatment is CHOEP, the second biomarkers may be selected from any one or more (e.g., one, two, three, four, five, ten, twenty, or all biomarkers) of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st.

For example, when the treatment is BeICHOP, the second biomarkers may be selected from any one or more (e.g., one, two, three, four, five, ten, twenty, or all biomarkers) of ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and/or hsa-miR-93_st.

For example, when the treatment is MTX, the second biomarkers may be selected from any one or more (e.g., one, two, three, four, five, ten, twenty, or all biomarkers) of ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and/or hsa-miR-936_st.

For example, when the treatment is doxorubicin, the second biomarkers may be selected from any one or more (e.g., one, two, three, four, five, ten, twenty, or all biomarkers) of ACA13_st, ACA48_x_st, U104_st, U55_st, U55_x_st, U74_x_st, hsa-miR-106a-star_st, hsa-miR-106b-star_st, hsa-miR-106b_st, hsa-miR-124_st, hsa-miR-1281_st, hsa-miR-1299_st, hsa-miR-140-3p_st, hsa-miR-195-star_st, hsa-miR-297_st, hsa-miR-29b-2-star_st, hsa-miR-33b-star_st, hsa-miR-342-3p_st, hsa-miR-342-5p_st, hsa-miR-432_st, hsa-miR-550-star_st, hsa-miR-629-star_st, hsa-miR-629_st, hsa-miR-652_st, hsa-miR-654-3p_st, hsa-miR-671-5p_st, hsa-miR-768-3p_st, hsa-miR-877-star_st, hsa-miR-93-star_st, and/or hsa-miR-93_st.

For example, when the treatment is bendamustine, the second biomarkers may be selected from any one or more (e.g., one, two, three, four, five, ten, twenty, or all biomarkers) of ACA11_st, ACA24_x_st, ACA7_s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and/or hsa-miR-93-star_st.

In several embodiments of the first aspect of the invention, a change in the level of expression (e.g., an increase or decrease) of hsa-miR-766_st and/or hsa-miR-34b_st indicates increased responsiveness of the cancer patient (e.g., a lymphoma patient, such as a patient with DLBCL) to the treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, and/or bendamustine). A change in the level of expression (e.g., an increase or decrease) of the one or more second biomarkers (e.g., one, two, three, four, five, ten, twenty, or all biomarkers for each respective therapy shown in Table 1) may also be used to indicate increased responsiveness of the patient to the treatment.

A second aspect of the invention features a method of predicting the responsiveness to adriamycin, bleomycin, vinblastine, and dacarbazine (ABVD); cyclophosphamide, vincristine, adriamycine (CHOP); and/or prednisolone treatment in a cancer patient (e.g., a lymphoma patient, such as a patient with DLBCL). The method includes determining a level of expression of hsa-miR-766_st in a biological sample (e.g., a tumor biopsy) from the patient, in which a change (e.g., an increase or a decrease) in the level of expression of hsa-miR-766_st indicates whether the patient will be responsive to the treatment.

In an embodiment of the second aspect of the invention, the method may further include determining the level of expression of one or more second biomarkers ((e.g., one, two, three, four, five, ten, twenty, or all biomarkers for each respective therapy shown in Table 1) in the biological sample (e.g., a tumor biopsy) from the cancer patient (e.g., a lymphoma patient, such as a patient with DLBCL) or in a second biological sample from the patient. In particular, a change (e.g., an increase or a decrease) in the level of expression of the one or more second biomarkers indicates that the patient is responsive to the treatment (e.g., ABVD, CHOP, and/or prednisolone).

For example, when the treatment is ABVD, the second biomarkers may be selected from any one or more (e.g., one, two, three, four, five, ten, twenty, or all biomarkers) of 14qII-14_st, 14qII-14_x_st, 14qII-1_st, 14qII-1_x_st, 14qII-22_x_st, 14qII-26_st, 14qII-26_x_st, HBII-85-26_st, HBII-85-6_x_st, U22_st, U48_st, U55_st, U55_x_st, hsa-miR-124_st, hsa-miR-125b-1-star_st, hsa-miR-127-3p_st, hsa-miR-1271_st, hsa-miR-1281_st, hsa-miR-1299_st, hsa-miR-134_st, hsa-miR-140-3p_st, hsa-miR-143_st, hsa-miR-145_st, hsa-miR-154_st, hsa-miR-195-star_st, hsa-miR-199a-5p_st, hsa-miR-199b-3p_st, hsa-miR-299-3p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-495_st, hsa-miR-629-star_st, hsa-miR-654-3p_st, hsa-miR-665_st, hsa-miR-671-5p_st, hsa-miR-758_st, and/or hsa-miR-923_st.

For example, when the treatment is CHOP, the second biomarkers may be selected from any one or more (e.g., one, two, three, four, five, ten, twenty, or all biomarkers) of ACA48_x_st, U55_x_st, hsa-miR-106b-star_st, hsa-miR-106b_st, hsa-miR-1181_st, hsa-miR-124_st, hsa-miR-1299_st, hsa-miR-25-star_st, hsa-miR-33b-star_st, hsa-miR-432_st, hsa-miR-551b-star_st, hsa-miR-629-star_st, hsa-miR-629_st, hsa-miR-652_st, hsa-miR-654-3p_st, hsa-miR-671-5p_st, hsa-miR-877-star_st, hsa-miR-93-star_st, and/or hsa-miR-93_st.

For example, when the treatment is prednisolone, the second biomarkers may be selected from any one or more (e.g., one, two, three, four, five, ten, twenty, or all biomarkers) of ACA18_x_st, ACA24_s_st, ACA24_x_st, ACA25_x_st, ACA3-2_st, ACA3_st, ACA40_x_st, ACA47_st, ACA48_st, ACA52_st, ACA54_st, ACA62_st, ACA7_s_st, ENSG00000199411_s_st, ENSG00000200394_st, ENSG00000200879_st, ENSG00000207002_st, HBII-180A_x_st, HBII-202_st, HBII-239_st, HBII-382_s_st, HBII-419_st, HBII-429_st, HBII-52-32_x_st, HBII-55_st, U104_st, U17a_st, U17b_st, U17b_x_st, U25_st, U26_st, U27_st, U28_st, U29_st, U3-2_s_st, U30_st, U31_st, U31_x_st, U33_st, U35A_st, U36A_x_st, U38A_st, U38B_st, U41_st, U46_st, U46_x_st, U49A_st, U50B_st, U50B_x_st, U50_st, U55_st, U56_st, U56_x_st, U57_st, U67_st, U67_x_st, U68_st, U68_x_st, U70_x_st, U73a_st, U78_s_st, U78_x_st, U83_st, U93_st, U95_st, hsa-miR-1181_st, hsa-miR-1207-5p_st, hsa-miR-1246_st, hsa-miR-1268_st, hsa-miR-1275_st, hsa-miR-17-star_st, hsa-miR-17_st, hsa-miR-195-star_st, hsa-miR-19b_st, hsa-miR-297_st, hsa-miR-571_st, hsa-miR-768-3p_st, hsa-miR-768-5p_st, hsa-miR-877-star_st, hsa-miR-92a-1-star_st, hsa-miR-92a-2-star_st, hsa-miR-92a_st, hsa-miR-938_st, and/or hsa-miR-939_st.

In several embodiments of the second aspect of the invention, a change in the level of expression (e.g., an increase or decrease) of hsa-miR-766_st indicates increased responsiveness of the cancer patient (e.g., a lymphoma patient, such as a patient with DLBCL) to the treatment (e.g., ABVD, CHOP, and/or prednisolone). In a further embodiment, a change in the level of expression (e.g., an increase or decrease) of the one or more second biomarkers (e.g., one, two, three, four, five, ten, twenty, or all biomarkers for each respective therapy shown in Table 1) indicates increased responsiveness of the patient to the treatment.

A third aspect of the invention features a method of predicting the responsiveness to gemcitabine and oxaliplatin (GemOx) treatment in a cancer patient (e.g., a lymphoma patient, such as a patient with DLBCL), which includes determining a level of expression of hsa-miR-34b_st in a biological sample (e.g., a tumor biopsy) from the patient, in which a change (e.g., an increase or a decrease) in the level of expression of hsa-miR-34b_st indicates whether the patient will be responsive to the treatment (e.g., GemOx).

In an embodiment of the third aspect of the invention, the method may further include determining the level of expression of one or more second biomarkers in the biological sample (e.g., a tumor biopsy) from the cancer patient (e.g., a lymphoma patient, such as a patient with DLBCL) or in a second biological sample from the patient. In particular, a change (e.g., an increase or a decrease) in the level of expression of the one or more second biomarkers may be used to indicate whether the patient will be responsive to treatment with GemOx. The one or more second biomarkers may be selected from any one or more (e.g., one, two, three, four, five, or all biomarkers) of hsa-miR-126_st, hsa-miR-153_st, hsa-miR-19b_st, hsa-miR-424-star_st, hsa-miR-491-3p_st, and hsa-miR-503_st.

In several embodiments of the third aspect of the invention, a change in the level of expression (e.g., an increase or decrease) of hsa-miR-34b_st indicates increased responsiveness of the cancer patient (e.g., a lymphoma patient, such as a patient with DLBCL) to the treatment (e.g., GemOx). In a further embodiment, a change in the level of expression (e.g., an increase or decrease) of the one or more second biomarkers (e.g., one, two, three, four, five, ten, twenty, or all biomarkers for each respective therapy shown in Table 1) may indicate increased responsiveness of the patient to the treatment (e.g., GemOx).

For any of the above aspects, the method may further include administering the treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, prednisolone, and/or GemOx) to the cancer patient (e.g., a lymphoma patient, such as a patient with DLBCL). The determining step may occur prior to the administration, substantially concurrent with the administration, or after the administration. In various embodiments, the determining step occurs multiple times and/or the administration step occurs multiple times. Each of the determining steps may occur prior to each of the administrations, substantially concurrent with each of the administrations, or after each of the administrations.

For any of the above aspects, the method may further include, prior to the determining step, providing the biological sample obtained from the patient. The biological sample may be formalin-fixed paraffin embedded (FFPE) tissue or fresh frozen tissue. In particular, the biological sample may be obtained from a tumor (e.g., a tumor biopsy). For example, the biological sample may include a tumor biopsy from a patient having suffered a relapse of diffuse large B-cell lymphoma after a first, second, or third line therapy.

A fourth aspect of the invention features a method of treating a cancer patient (e.g., a lymphoma patient, such as a patient with DLBCL), in which the patient has been determined to be responsive to a treatment based on the level of expression of hsa-miR-766_st and/or hsa-miR-34b_st. The method includes administering a treatment selected from e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, and/or bendamustine. In an embodiment, prior to the administration, the patient was determined to have a level of expression of hsa-miR-766_st and/or hsa-miR-34b_st that is statistically different from the level of expression of hsa-miR-766_st and/or hsa-miR-34b_st in a control (e.g., expression levels of a population median or expression levels determined from a control sample, such as a sample from a patient known to respond to the subject therapy). In particular, the statistical difference indicates the responsiveness of the patient to the treatment. In another embodiment, prior to the administration, the patient was determined to be responsive to the treatment based on a level of expression of one or more second biomarkers (e.g., one, two, three, four, five, ten, twenty, or all biomarkers for each respective therapy shown in Table 1). For example, the patient was determined to have a level of expression of the second biomarkers that is statistically different from the level of expression of the second biomarkers in a control, such that the statistical difference indicates the responsiveness of the patient to the treatment.

For example, when the treatment is COPE, the second biomarkers may be selected from any one or more (e.g., one, two, three, four, five, ten, twenty, or all biomarkers) of ACA48_x_st, ENSG00000202498_x_st, HBII-85-26_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and/or hsa-miR-93-star_st.

For example, when the treatment is MBVP, the second biomarkers may be selected from any one or more (e.g., one, two, three, four, five, ten, twenty, or all biomarkers) of ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and/or hsa-miR-93-star_st.

For example, when the treatment is DHAP, the second biomarkers may be selected from any one or more (e.g., one, two, three, four, five, ten, twenty, or all biomarkers) of ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2_s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and/or hsa-miR-938_st.

For example, when the treatment is ICE, the second biomarkers may be selected from any one or more (e.g., one, two, three, four, five, ten, twenty, or all biomarkers) of 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and/or hsa-miR-938_st.

For example, when the treatment is VIM, the second biomarkers may be selected any one or more (e.g., one, two, three, four, five, ten, twenty, or all biomarkers) of from ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, when the treatment is MIME, the second biomarkers may be selected from any one or more (e.g., one, two, three, four, five, ten, twenty, or all biomarkers) of ACA10_s_st, ACA11_st, ACA13_st, ACA18_x_st, ACA21_st, ACA40_x_st, ACA48_x_st, ACA51_x_st, ACA55_st, ACA61_st, ACA7_s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, when the treatment is ESHAP, the second biomarkers may be selected from any one or more (e.g., one, two, three, four, five, ten, twenty, or all biomarkers) of ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and/or hsa-miR-768-5p_st.

For example, when the treatment is CBV, the second biomarkers may be selected from any one or more (e.g., one, two, three, four, five, ten, twenty, or all biomarkers) of ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and/or hsa-miR-938_st.

For example, when the treatment is EPOCH, the second biomarkers may be selected from any one or more (e.g., one, two, three, four, five, ten, twenty, or all biomarkers) is selected from any one or more of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st.

For example, when the treatment is FCM, the second biomarkers may be selected from any one or more (e.g., one, two, three, four, five, ten, twenty, or all biomarkers) of ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and/or hsa-miR-92b_st.

For example, when the treatment is CVAD-A, the second biomarkers may be selected from any one or more (e.g., one, two, three, four, five, ten, twenty, or all biomarkers) of ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and/or hsa-miR-92a-2-star_st.

For example, when the treatment is CVAD-B, the second biomarkers may be selected from any one or more (e.g., one, two, three, four, five, ten, twenty, or all biomarkers) of ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_st, hsa-miR-663b_st, hsa-miR-768-5p_st, and/or hsa-miR-92a_st.

For example, when the treatment is MINE, the second biomarkers may be selected from any one or more (e.g., one, two, three, four, five, ten, twenty, or all biomarkers) of 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and/or hsa-miR-938_st.

For example, when the treatment is MOPP, the second biomarkers may be selected from any one or more (e.g., one, two, three, four, five, ten, twenty, or all biomarkers) of ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and/or hsa-miR-92b_st.

For example, when the treatment is CHOEP, the second biomarkers may be selected from any one or more (e.g., one, two, three, four, five, ten, twenty, or all biomarkers) of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st.

For example, when the treatment is BeICHOP, the second biomarkers may be selected from any one or more (e.g., one, two, three, four, five, ten, twenty, or all biomarkers) of ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and/or hsa-miR-93_st.

For example, when the treatment is MTX, the second biomarkers may be selected from any one or more (e.g., one, two, three, four, five, ten, twenty, or all biomarkers) of ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and/or hsa-miR-936_st.

For example, when the treatment is doxorubicin, the second biomarkers may be selected from any one or more (e.g., one, two, three, four, five, ten, twenty, or all biomarkers) of ACA13_st, ACA48_x_st, U104_st, U55_st, U55_x_st, U74_x_st, hsa-miR-106a-star_st, hsa-miR-106b-star_st, hsa-miR-106b_st, hsa-miR-124_st, hsa-miR-1281_st, hsa-miR-1299_st, hsa-miR-140-3p_st, hsa-miR-195-star_st, hsa-miR-297_st, hsa-miR-29b-2-star_st, hsa-miR-33b-star_st, hsa-miR-342-3p_st, hsa-miR-342-5p_st, hsa-miR-432_st, hsa-miR-550-star_st, hsa-miR-629-star_st, hsa-miR-629_st, hsa-miR-652_st, hsa-miR-654-3p_st, hsa-miR-671-5p_st, hsa-miR-768-3p_st, hsa-miR-877-star_st, hsa-miR-93-star_st, and/or hsa-miR-93_st.

For example, when the treatment is bendamustine, the second biomarkers may be selected from any one or more (e.g., one, two, three, four, five, ten, twenty, or all biomarkers) of ACA11_st, ACA24_x_st, ACA7s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and/or hsa-miR-93-star_st.

In several embodiments of the fourth aspect of the invention, a change in the level of expression (e.g., an increase or decrease) of hsa-miR-766_st and/or hsa-miR-34b_st indicates increased responsiveness of the patient (e.g., a lymphoma patient, such as a patient with DLBCL) to the treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, and/or bendamustine). In a further embodiment, a change in the level of expression (e.g., an increase or decrease) of the one or more second biomarkers (e.g., one, two, three, four, five, ten, twenty, or all biomarkers for each respective therapy shown in Table 1) indicates increased responsiveness of the patient to the treatment.

A fifth aspect of the invention features a method of treating a cancer patient (e.g., a lymphoma patient, such as a patient with DLBCL), in which the patient has been determined to be responsive to a treatment based on the level of expression of hsa-miR-766_st. The method includes administering a treatment selected from, e.g., adriamycin, bleomycin, vinblastine, and dacarbazine (ABVD); cyclophosphamide, vincristine, adriamycine (CHOP); and/or prednisolone. In an embodiment, prior to the administration, the patient was determined to have a level of expression of hsa-miR-766_st that is statistically different from the level of expression of hsa-miR-766_st in a control (e.g., expression levels of a population median or expression levels determined from a control sample, such as a sample from a patient known to respond to the subject therapy). In particular, the statistical difference indicates the responsiveness of the patient to the treatment. In another embodiment, prior to the administration, the patient was determined to be responsive to the treatment based on a level of expression of one or more second biomarkers (e.g., one, two, three, four, five, ten, twenty, or all biomarkers for each respective therapy shown in Table 1). For example, the patient was determined to have a level of expression of the second biomarkers that is statistically different from the level of expression of the second biomarkers in a control, such that the statistical difference indicates the responsiveness of the patient to the treatment.

For example, when the treatment is ABVD, the second biomarkers may be selected from any one or more (e.g., one, two, three, four, five, ten, twenty, or all biomarkers) of 14qII-14_st, 14qII-14_x_st, 14qII-1_st, 14qII-1_x_st, 14qII-22_x_st, 14qII-26_st, 14qII-26_x_st, HBII-85-26_st, HBII-85-6_x_st, U22_st, U48_st, U55_st, U55_x_st, hsa-miR-124_st, hsa-miR-125b-1-star_st, hsa-miR-127-3p_st, hsa-miR-1271_st, hsa-miR-1281_st, hsa-miR-1299_st, hsa-miR-134_st, hsa-miR-140-3p_st, hsa-miR-143_st, hsa-miR-145_st, hsa-miR-154_st, hsa-miR-195-star_st, hsa-miR-199a-5p_st, hsa-miR-199b-3p_st, hsa-miR-299-3p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-495_st, hsa-miR-629-star_st, hsa-miR-654-3p_st, hsa-miR-665_st, hsa-miR-671-5p_st, hsa-miR-758_st, and/or hsa-miR-923_st.

For example, when the treatment is CHOP, the second biomarkers may be selected from any one or more (e.g., one, two, three, four, five, ten, twenty, or all biomarkers) of ACA48_x_st, U55_x_st, hsa-miR-106b-star_st, hsa-miR-106b_st, hsa-miR-1181_st, hsa-miR-124_st, hsa-miR-1299_st, hsa-miR-25-star_st, hsa-miR-33b-star_st, hsa-miR-432_st, hsa-miR-551b-star_st, hsa-miR-629-star_st, hsa-miR-629_st, hsa-miR-652_st, hsa-miR-654-3p_st, hsa-miR-671-5p_st, hsa-miR-877-star_st, hsa-miR-93-star_st, and/or hsa-miR-93_st.

For example, when the treatment is prednisolone, the second biomarkers may be selected from any one or more (e.g., one, two, three, four, five, ten, twenty, or all biomarkers) of ACA18_x_st, ACA24_s_st, ACA24_x_st, ACA25_x_st, ACA3-2_st, ACA3_st, ACA40_x_st, ACA47_st, ACA48_st, ACA52_st, ACA54_st, ACA62_st, ACA7_s_st, ENSG00000199411_s_st, ENSG00000200394_st, ENSG00000200879_st, ENSG00000207002_st, HBII-180A_x_st, HBII-202_st, HBII-239_st, HBII-382_s_st, HBII-419_st, HBII-429_st, HBII-52-32_x_st, HBII-55_st, U104_st, U17a_st, U17b_st, U17b_x_st, U25_st, U26_st, U27_st, U28_st, U29_st, U3-2_s_st, U30_st, U31_st, U31_x_st, U33_st, U35A_st, U36A_x_st, U38A_st, U38B_st, U41_st, U46_st, U46_x_st, U49A_st, U50B_st, U50B_x_st, U50_st, U55_st, U56_st, U56_x_st, U57_st, U67_st, U67_x_st, U68_st, U68_x_st, U70_x_st, U73a_st, U78_s_st, U78_x_st, U83_st, U93_st, U95_st, hsa-miR-1181_st, hsa-miR-1207-5p_st, hsa-miR-1246_st, hsa-miR-1268_st, hsa-miR-1275_st, hsa-miR-17-star_st, hsa-miR-17_st, hsa-miR-195-star_st, hsa-miR-19b_st, hsa-miR-297_st, hsa-miR-571_st, hsa-miR-768-3p_st, hsa-miR-768-5p_st, hsa-miR-877-star_st, hsa-miR-92a-1-star_st, hsa-miR-92a-2-star_st, hsa-miR-92a_st, hsa-miR-938_st, and/or hsa-miR-939_st.

In several embodiments of the fifth aspect of the invention, a change in the level of expression (e.g., an increase or decrease) of hsa-miR-766_st indicates increased responsiveness of the cancer patient (e.g., a lymphoma patient, such as a patient with DLBCL) to the treatment (e.g., ABVD, CHOP, and/or prednisolone).

A sixth aspect of the invention features a method of treating a cancer patient (e.g., a lymphoma patient, such as a patient with DLBCL), in which the patient has been determined to be responsive to a treatment based on the level of expression of hsa-miR-34b_st. The method includes administering GemOx treatment. In an embodiments, prior to the administration, the patient was determined to have a level of expression of hsa-miR-34b_st that is statistically different from the level of expression of hsa-miR-34b_st in a control (e.g., expression levels of a population median or expression levels determined from a control sample, such as a sample from a patient known to respond to the subject therapy). In another embodiment, prior to the administration, the patient was determined to be responsive to the treatment based on a level of expression of one or more second biomarkers (e.g., one, two, three, four, five, ten, twenty, or all biomarkers for each respective therapy shown in Table 1). For example, the patient was determined to have a level of expression of the second biomarkers that is statistically different from the level of expression of the second biomarkers in a control (e.g., expression levels of a population median or expression levels determined from a control sample, such as a sample from a patient known to respond to the subject therapy). In particular, the statistical difference indicates the responsiveness of the patient to the treatment. In preferred embodiments, the one or more second biomarkers may be selected from any one or more of hsa-miR-126_st, hsa-miR-153_st, hsa-miR-19b_st, hsa-miR-424-star_st, hsa-miR-491-3p_st, and hsa-miR-503_st.

For any of the above aspects, the level of expression of one, at least two, at least three, at least four, at least five, or each of the second biomarkers was determined in the patient.

In several embodiments of the method of the first, second, third, fourth, fifth, and sixth aspects of the invention, the method may further include administering one or more additional therapies to the patient. The one or more additional therapies may be administered concurrently with administration of the treatment, separately from administration of the treatment, prior to administration of the treatment, or after administration of the treatment. For example, the one or more additional therapies may include one or more additional therapeutic agents (e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, prednisolone, and/or GemOx), surgery, and/or radiation therapy. The treatment may be administered to the patient intravenously, orally, intraperitoneally, intramuscularly, topically, rectally, cutaneously, subcutaneously, nasally, intracerebroventricularly, intraparenchymally, intrathecally, intracranially, ocularly, via inhalation, or through the skin. Preferably, the treatment is administered to the patient intravenously. In various embodiments, the method may further include administering the treatment two or more times, such that treatment is administered within a 24 hour period or once daily (e.g., once daily for up to five days). In certain embodiments, administration of the treatment is repeated 30 days after the completion of the previous administration. Treatment may be administered to the patient once weekly (e.g., in six to eight week cycles), once every other week, or once every three weeks (e.g., in six to eight three week cycles). In various embodiments, administration of the treatment is repeated for at least 12 to 60 months.

For any of the above aspects, the treatment may be administered in a dosage form (e.g., a solid, such as a table, capsule, or pill, or a liquid). In preferred embodiments, the patient is a human. In certain embodiments, the patient has suffered a relapse of diffuse large B-cell lymphoma, e.g., prior to the determining step.

For any of the above aspects, the level of expression of the biomarker (e.g., the biomarkers for each respective therapy shown in Table 1) in the cancer patient (e.g., a lymphoma patient, such as a patient with DLBCL) may be significantly different (e.g., higher or lower) than in the control (e.g., expression levels of a population median or expression levels determined from a control sample, such as a sample from a patient known to respond to the subject therapy). The level of expression of the biomarker may be determined by detecting the level of microRNA in the biological sample (e.g., a tumor biopsy), such as, e.g., in a cell (e.g., a cancer cell). The method may further include, prior to the determining, amplifying and/or reverse transcribing nucleic acid molecules (e.g., RNA or DNA) corresponding to hsa-miR-766 and/or hsa-miR-34b and/or one or more second biomarkers (e.g., the biomarkers shown in Table 1) in the biological sample. The product of the amplification, the product of the reverse transcription, or the biological sample itself may be cDNA. The levels of expression of the biomarkers may be determined using, e.g., a device, such as a microarray (e.g., an array containing full-length cDNAs complementary to an RNA or cDNA fragments (e.g., a solid support microarray)) or sequencing (e.g., 454 pyrosequencing, Illumina sequencing by synthesis, SOLiD sequencing, Ion Torrent sequencing, or PacBio RS sequencing). The microarray may contain a plurality of nucleic acid probes (e.g., the sequences shown in Table 2), such that each of the nucleic acid probes is configured to hybridize to a target RNA molecule or a target cDNA molecule (e.g., a RNA or cDNA molecule that is identical or complementary to all or a portion of the nucleic acid sequence of at least one of the biomarkers). In particular embodiments, each of the nucleic acid probes (e.g., a nucleic acid probe of, e.g., about 5 nt, 10 nt, 15 nt, 20 nt, 25 nt, 30 nt, 50 nt, 75 nt, 100 nt, 150 nt, or 200 nt in length) includes a sequence having at least, e.g., 75%, 85%, 90%, 95%, 98%, 99%, or 100% sequence identity to a continuous sequence within the target RNA molecule or the target cDNA molecule (e.g., the biomarkers shown in Table 1).

In various embodiments of any aspect of the invention, the level of expression of hsa-miR-766_st may be determined using a probe capable of hybridizing to a nucleic acid molecule having the sequence of SEQ ID NO. 246. In further embodiments of any aspect of the invention, the level of expression of hsa-miR-34b_st may be determined using a probe capable of hybridizing to a nucleic acid molecule having the sequence of SEQ ID NO. 185. The nucleic acid sequence of the probe for hsa-miR-766_st or hsa-miR-34b_st may include at least 15 continuous nucleotides (e.g., at least 16, at least 17, at least 18, at least 19, at least 20, at least 21, at least 22, at least 23, at least 24, at least 25, or at least 30) and at least 85% (e.g., at least 90%, at least 95%, or 100%) sequence identity to a sequence complementary to SEQ ID NO. 246 or SEQ ID NO. 185, respectively. The method may further include that the level of expression of the biomarkers (e.g., one or more of the biomarkers of Table 1) is determined using a probe capable of hybridizing to a nucleic acid molecule including the nucleic acid sequence of the biomarkers (e.g., a nucleic acid sequence including at least 15 (e.g., at least 16, at least 17, at least 18, at least 19, at least 20, at least 21, at least 22, at least 23, at least 24, at least 25, or at least 30) continuous nucleotides and at least 85% (e.g., at least 90%, at least 95%, or 100%) sequence identity to a sequence complementary to SEQ ID NOs. 1-263).

For any of the above aspects, the method may further include converting the level of expression of the biomarker into a mean score for the treatment (e.g., the mean score identifies the responsiveness of the patient to the treatment). In various embodiments of each of the methods, a change in the level of expression (e.g., an increase or decrease) of one or more of the second biomarkers (e.g., the biomarkers shown in Table 1) indicates increased responsiveness of the cancer patient (e.g., a lymphoma patient, such as a patient with DLBCL) to the treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, prednisolone, and/or GemOx).

### DEFINITIONS

The terms “responsive” and “responsiveness,” as used herein with respect to a patient's responsiveness to a treatment, e.g., treatment with a compound, such as an anti-cancer agent (e.g., doxorubicin, bendamustine, prednisolone, cyclophosphamide, MTX, vincristine, or Romidepsin), or combination treatment (CVP, COPE, MBVP, DHAP, ICE, VIM, MIME, ESHAP, ABVD, BEACOPP, CBV, CEPP, COPP, EPOCH, FCM, GemOx, CVAD-A, CVAD-B, MINE, MOPP, CHOP, CHOEP, or BeICHOP), or treatment with radiation, refer to the likelihood that the treatment has (e.g., induces) a desired effect, or alternatively refers to the strength of a desired effect caused or induced by the treatment in a cell, a tumor, or patient (e.g., a mammal, such as a human). For example, the desired effect can include inhibition of the growth of a cell, e.g., a cancer cell, in vitro or in a tumor, person, or living organism by more than 10%, 20%, 30%, 40%, 50%, 60%, 70%, 80%, 90%, or 100% relative to the growth of a cell not exposed to the treatment. The desired effect can also include reduction in tumor mass by, e.g., about 10%, 20%, 30%, 40%, 50%, 60%, 70%, 80%, 90%, or 100%. Responsiveness to treatment may be determined by a cell-based assay that measures the growth of treated cells as a function of the cells' absorbance of an incident light beam as used to perform the NCI60 assays described herein. In this example, lesser absorbance indicates lesser cell growth, and thus, sensitivity to the treatment. A greater reduction in growth indicates more sensitivity to the treatment. According to the invention, “responsiveness” is a measure of the sensitivity or resistance of a patient to a treatment, e.g., for cancer, for example, by treatment with a treatment or combination treatment (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP).

By “biomarker” and “biomarker gene” is meant a gene (e.g., a protein-coding DNA, mRNA, microRNA, or noncoding RNA) in a cell, the expression of which correlates to responsiveness (e.g., sensitivity or resistance) of the cell (and thus the patient containing the cell or from which the cell was obtained) to a treatment (e.g., exposure to a compound or combination treatment of the invention).

The terms “expression level” and “level of expression,” as used herein, refer to the amount of a gene product in a cell, tissue, biological sample, organism, or patient, e.g., amounts of DNA, RNA, or proteins, amounts of modifications of DNA, RNA, or protein, such as splicing, phosphorylation, acetylation, or methylation, or amounts of activity of DNA, RNA, or proteins associated with a given gene.

“Complement” of a nucleic acid sequence or a “complementary” nucleic acid sequence as used herein refers to an oligonucleotide which is in “antiparallel association” when it is aligned with the nucleic acid sequence such that the 5′ end of one sequence is paired with the 3′ end of the other. Nucleotides and other bases may have complements and may be present in complementary nucleic acids. Bases not commonly found in natural nucleic acids that may be included in the nucleic acids of the invention include, for example, inosine and 7-deazaguanine. “Complementarity” may not be perfect; stable duplexes of complementary nucleic acids may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

When complementary nucleic acid sequences form a stable duplex, they are said to be “hybridized” or to “hybridize” to each other or it is said that “hybridization” has occurred. Nucleic acids are referred to as being “complementary” if they contain nucleotides or nucleotide homologues that can form hydrogen bonds according to Watson-Crick base-pairing rules (e.g., G with C, A with T or A with U) or other hydrogen bonding motifs such as for example diaminopurine with T, 5-methyl C with G, 2-thiothymidine with A, inosine with C, pseudoisocytosine with G, etc. Anti-sense RNA may be complementary to other oligonucleotides, e.g., mRNA.

“Gene” as used herein indicates a coding or noncoding gene whose activity can be determined by measuring a gene product, e.g., RNA or protein. Examples include protein coding genes, microRNAs, small nuclear RNAs and other RNAs with catalytic, regulatory or coding properties.

“Microarray” as used herein means a device employed by any method that quantifies one or more subject oligonucleotides, e.g., DNA or RNA, or analogues thereof, at a time. One exemplary class of microarrays consists of DNA probes attached to a glass or quartz surface. For example, many microarrays, including those made by Affymetrix, use several probes for determining the expression of a single gene. The DNA microarray may contain oligonucleotide probes that may be, e.g., full-length cDNAs complementary to an RNA or cDNA fragments that hybridize to part of an RNA. Exemplary RNAs include mRNA, miRNA, and miRNA precursors. Exemplary microarrays also include a “nucleic acid microarray” having a substrate-bound plurality of nucleic acids, hybridization to each of the plurality of bound nucleic acids being separately detectable. The substrate may be solid or porous, planar or non-planar, unitary or distributed. Exemplary nucleic acid microarrays include all of the devices so called in Schena (ed.), DNA Microarrays: A Practical Approach (Practical Approach Series), Oxford University Press (1999); Nature Genet. 21(1)(suppl.):1-60 (1999); Schena (ed.), Microarray Biochip: Tools and Technology, Eaton Publishing Company/BioTechniques Books Division (2000). Additionally, exemplary nucleic acid microarrays include substrate-bound plurality of nucleic acids in which the plurality of nucleic acids are disposed on a plurality of beads, rather than on a unitary planar substrate, as is described, inter alia, in Brenner et al., Proc. Natl. Acad. Sci. USA 97(4):1665-1670 (2000). Examples of nucleic acid microarrays may be found in U.S. Pat. Nos. 6,391,623, 6,383,754, 6,383,749, 6,380,377, 6,379,897, 6,376,191, 6,372,431, 6,351,712 6,344,316, 6,316,193, 6,312,906, 6,309,828, 6,309,824, 6,306,643, 6,300,063, 6,287,850, 6,284,497, 6,284,465, 6,280,954, 6,262,216, 6,251,601, 6,245,518, 6,263,287, 6,251,601, 6,238,866, 6,228,575, 6,214,587, 6,203,989, 6,171,797, 6,103,474, 6,083,726, 6,054,274, 6,040,138, 6,083,726, 6,004,755, 6,001,309, 5,958,342, 5,952,180, 5,936,731, 5,843,655, 5,814,454, 5,837,196, 5,436,327, 5,412,087, 5,405,783, the disclosures of which are incorporated herein by reference in their entireties.

Exemplary microarrays may also include “peptide microarrays” or “protein microarrays” having a substrate-bound plurality of polypeptides, the binding of a oligonucleotide, a peptide, or a protein to each of the plurality of bound polypeptides being separately detectable. Alternatively, the peptide microarray, may have a plurality of binders, including but not limited to monoclonal antibodies, polyclonal antibodies, phage display binders, yeast 2 hybrid binders, aptamers, which can specifically detect the binding of specific oligonucleotides, peptides, or proteins. Examples of peptide arrays may be found in WO 02/31463, WO 02/25288, WO 01/94946, WO 01/88162, WO 01/68671, WO 01/57259, WO 00/61806, WO 00/54046, WO 00/47774, WO 99/40434, WO 99/39210, WO 97/42507 and U.S. Pat. Nos. 6,268,210, 5,766,960, 5,143,854, the disclosures of which are incorporated herein by reference in their entireties.

As used herein, the term “percent (%) sequence identity” refers to the percentage of amino acid (or nucleic acid) residues of a candidate sequence, e.g., a probe or primer of the invention, that are identical to the amino acid (or nucleic acid) residues of a reference sequence, e.g., a biomarker sequence of the invention, after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. In particular embodiments, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence, aligned for comparison purposes, is at least 30%, e.g., at least 40%, e.g., at least 50%, 60%, 70%, 80%, 90%, or 100% of the length of the reference sequence. When a position in the candidate sequence is occupied by the same amino acid residue as the corresponding position in the reference sequence, then the molecules are identical at that position.

As used herein, the term “substantially identical” refers to a polypeptide or polynucleotide sequence that has the same polypeptide or polynucleotide sequence, respectively, as a reference sequence (e.g., a biomarker sequence of the invention), or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids or more (e.g., the full-length sequence). For nucleic acids, the length of comparison sequences will generally be at least 5, 10, 11, 12, 13, 14, 15, 16, 17, 18, 19, 20, 21, 22, 23, 24, 25, 50, 75, 100, 200, 300, 400, 500, 1000, 2000, 3000, 4000, 5000 or more contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, insertions, deletions, and other modifications.

The term “biological sample,” as used herein, refers to any specimen (e.g., tissue, fluid, and cells (e.g., a tissue sample obtained by biopsy) taken from a patient. Preferably, the sample is taken from a portion of the body affected by or at risk of cancer, such as lymphoma (e.g., DLBCL). Biopsy (e.g., tumor biopsy) may involve fine needle aspiration biopsy, core needle biopsy (e.g., stereotactic core needle biopsy, vacuum-assisted core biopsy, or magnetic resonance imaging (MRI) guided biopsy), or surgical biopsy (e.g., incisional biopsy or excisional biopsy). The sample may undergo additional purification and processing, for example, to remove cell debris and other unwanted molecules. Additional processing may further involve amplification, e.g., using PCR (e.g., RT-PCR). The standard methods of sample purification, such as removal of unwanted molecules, are known in the art.

The terms “patient” and “subject,” as used interchangeably herein, refer to any animal (e.g., a mammal, e.g., a human). A patient to be treated or tested for responsiveness to a treatment according to the methods described herein may be one who has been diagnosed with a cancer, such as lymphoma (e.g., DLBCL, cutaneous T-cell lymphoma, peripheral T-cell lymphoma, Hodgkin's lymphoma, non-Hodgkin's lymphoma, Waldenstrom's macroglobulinemia, or lymphocytic lymphoma.). Diagnosis may be performed by any method or techniques known in the art, such as self-exam, x-ray, MRI, or biopsy. A patient may have been identified using techniques and methods known in the art. To minimize exposure of a patient to drug treatments that may not be therapeutic, the patient may be determined to be either responsive or non-responsive to a specific treatment regimen (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP) according to the methods of the invention. A “control” or “control subject” refers, for example, to a subject not suffering from or not susceptible to a disease, disorder, or condition treatable or diagnosable with the methods described herein (e.g., a subject without a cancer, such as lymphoma (e.g., DLBCL)), or to a population median (e.g., a median level measured in a set of subjects not suffering from or not susceptible to a disease, disorder, or condition treatable or diagnosable with the methods described herein (e.g., a subject not having cancer, such as lymphoma (e.g., DLBCL))).

“Treatment,” “medical treatment,” to “treat,” and “therapy” mean administering or exposing a subject, a cell, or a tumor to, e.g., a compound, such as an anti-cancer agent (e.g., a drug, a protein, an antibody, a nucleic acid, a chemotherapeutic agent, or a radioactive agent), or to some other form of medical intervention used to treat or prevent a disease, disorder, or condition (e.g., surgery, cryotherapy, radiation therapy, or combinations thereof). In certain embodiments, the disease to be treated is lymphoma or symptoms of lymphoma (e.g., DLBCL). Radiation therapy includes the administration of a radioactive agent to a subject, or exposure of a subject to radiation. The radiation may be generated from sources such as particle accelerators and related medical devices or agents that emit, e.g., X-radiation, gamma radiation, or electron (Beta radiation) beams. A treatment may further include surgery, e.g., to remove a tumor from a subject or living organism.

The term “diffuse large B-cell lymphoma” or “DLBCL” as used herein refers to a class of lymphoma distinguished as a molecularly heterogeneous condition. Traditionally, gene expression profiling has identified at least two molecular subtypes of DLBCL that are biologically and clinically distinct (Rosenwald et al, N. Engl. J. Med., 346: 1937-47 (2002); Alizadeh et al, Nature, 403: 503-1 1 (2000)). The germinal center B cell-like (GCB) DLBCL subtype likely arises from normal germinal center B cells, whereas the activated B cell-like (ABC) DLBCL subtype may arise from a post-germinal center B cell that is blocked during plasmacytic differentiation. Certain genetic alterations are more common in specific subtypes: GCB DLBCLs have recurrent translocations, whereas ABC DLBCLs more often have recurrent trisomy 3 and deletion of the INK4a ARF locus as well as constitutive activation of the anti-apoptotic NF-kB signalling pathway (Rosenwald et al, N. Engl. J. Med., 346: 1937-47 (2002); Bea et al, Blood, 106: 3183-90 (2005); Tagawa et al, Blood, 106: 1770-77 (2005); Davis et al, J. Exp. Med., 194:1861-74 (2001); Ngo et al, Nature, 441: 106-10 (2006); Lenz et al, Science, 319: 1676-79 (2008)). The current standard of care for the treatment of diffuse large B cell lymphoma (DLBCL) includes anthracycline-based chemotherapy regimens such as CHOP in combination with the administration of the anti-CD20 monoclonal antibody Rituximab. This combination regimen (R-CHOP) can treat more than 50% of patients with DLBCL and has improved the overall survival of DLBCL patients by 10-15% (Coiffier et al, N. Engl. J. Med., 346: 235-42 (2002)).

The terms “cancer” and “cancerous” refer to or describe the physiological condition in mammals that is typically characterized by unregulated cell proliferation. Examples of cancer include but are not limited to, lymphoma (such as DLBCL), carcinoma, blastoma, sarcoma, and leukemia. More particular examples of such cancers include B-cell lymphoma (including low grade/follicular non-Hodgkin's lymphoma (NHL), such as small lymphocytic (SL) NHL, intermediate grade/follicular NHL, intermediate grade diffuse NHL, high grade immunoblastic NHL, high grade lymphoblastic NHL, high grade small non-cleaved cell NHL, and bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, post-transplant lymphoproliferative disorder (PTLD), abnormal vascular proliferation associated with phakomatoses, edema (such as that associated with brain tumors), Meigs' syndrome, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma, and various types of head and neck cancer.

As used herein, “about” refers to an amount ±10 of the recited value.

As used herein, “a” or “an” means “at least one” or “one or more” unless otherwise indicated. In addition, the singular forms “a”, “an”, and “the” include plural referents unless the context clearly dictates otherwise.

Other features and advantages of the invention will be apparent from the following Detailed Description, the drawings, and the claims.

### BRIEF DESCRIPTION OF THE DRAWINGS

FIG. 1 is a graph showing the correlation between predicted sensitivity of DLBCL patients to CHOP/CHOEP (CHOP in combination with etoposide) and response to treatment (CC=0.24, P=0.006) using the biomarkers listed in Table 1 for treatment with CHOP/CHOEP. CR=Complete Remission, CRu=Complete Remission unconfirmed, PR=Partial Remission, PD=Progressive Disease, Dead=dead before response evaluation. A Wilcoxon rank test comparing CR to all other responses gives a p-value of 0.03. The pre-specified cutoff is shown with an orange line. The patient in the last column with a high predicted sensitivity (prediction score 82) died from a relapse within 148 days of diagnosis.

FIG. 2 is a graph showing overall survival after second and third line therapy among thirteen patients with disease relapse. The top line shows the Kaplan-Meier curve of five patients that are predicted sensitive to second and third line treatment received, and the bottom line shows the eight patients predicted resistant.

### DETAILED DESCRIPTION OF THE INVENTION

We have discovered that expression levels of certain biomarkers (e.g., the biomarkers shown in Table 1, in particular hsa-miR-766_st and/or hsa-miR-34b_st) may be used to predict the responsiveness of a cancer patient, e.g., a lymphoma patient, such as a patient with DLBCL, to a diversity of monotherapies and combination therapies, e.g., cyclophosphamide and vincristine (CVP); cyclophosphamide, vincristine, and etoposide (COPE); methotrexate, carmustine, and teniposide (MBVP); methotrexate (MTX); dexamethasone, ara-c, and cisplatin (DHAP); ifosfamide, carboplatin, and etoposide (ICE); etoposide, ifosfamide, and methotrexate (VIM); methyl-GAG, ifosfamide, methotrexate, and etoposide (MIME); adriamycin, bleomycin, and vinblastine (ABVD); etoposide, cytarabine, methylprednisolone, and cisplatin (ESHAP); bleomycin, etoposide, adriamycin, cyclophosphamide, vincristine, and procarbazine (BEACOPP); cyclophosphamide, vincristine, and procarbazine (CBV); cyclophosphamide, vincristine, and procarbazine (COPP); cyclophosphamide, etoposide, vincristine, and doxorubicine (EPOCH); fludarabine, cyclophosphamide, and mitoxantrone (FCM); gemcitabine and oxaliplatin (GemOx); cyclophosphamide, vincristine, doxorubicin, and dexamethasone (CVAD-A); methotrexate and cytarabine (CVAD-B); ifosfamide, mitoxantrone, and etoposide (MINE); mechlorethamine, vincristine, and procarbazine (MOPP); cyclophosphamide, vincristine, and adriamycine (CHOP); cyclophosphamide, vincristine, adriamycine, and etoposide (CHOEP); cyclophosphamide, vincristine, adriamycine, and belinostat (BeICHOP); doxorubicin; and/or bendamustine. The invention features sets of biomarkers (e.g., microRNAs or miRNAs) that enable the prediction of the responsiveness of a cancer patient, e.g., a lymphoma patient, such as a patient with DLBCL, to the above mentioned treatments at various stages of disease progression and a different times during the treatment process (e.g., before or after first line treatment, second line treatment, and/or third line treatment).

In particular, the invention provides methods useful for predicting whether a patient is likely to be responsive to a specific cancer treatment by, e.g., determining the expression level (e.g., mRNA or protein expression level) of one or more biomarkers (e.g., hsa-miR-766_st and/or hsa-miR-34b_st) in a biological sample (e.g., a tumor biopsy) obtained from the subject. The expression levels may then be compared to the expression levels of a control (e.g., expression levels of a population median or expression levels determined from a control sample, such as a sample from a patient known to respond to the subject therapy) to predict the patient's responsiveness to administration of various combination treatments. Biomarkers of the invention, e.g., hsa-miR-766_st and hsa-miR-34b_st, may also be used to predict the responsive of a cancer patient, e.g., a lymphoma patient, such as a patient with DLBCL, to a monotherapy, e.g, therapy with methotrexate (MTX), doxorubicin, or bendamustine. Furthermore, a single biomarker, e.g., hsa-miR-766_st, may be used to predict patient responsiveness to combination therapy or monotherapy, e.g., therapy with adriamycin, bleomycin, vinblastine, and dacarbazine (ABVD); cyclophosphamide, vincristine, adriamycine (CHOP); or prednisolone. Other single biomarkers, e.g, hsa-miR-34b_st, may be used to predict the response of a cancer patient, e.g., a lymphoma patient, such as a patient with DLBCL, to a combination treatment, e.g., therapy with gemcitabine and oxaliplatin (GemOx), administered for lymphoma (e.g., DLBCL). The invention also features the use of at least a second biomarker, e.g., one or more of the biomarkers listed in Table 1, for the respective combination therapies or monotherapies to predict a cancer patient's (e.g., a DLBCL patient) responsiveness to one or more of the indicated treatments.

TABLE 1
microRNAs used to predict treatment response for the drug and drug combinations
listed, which are routinely used in the treatment of lymphoma.

| Treatment | microRNAs used to predict treatment response |
|---|---|
| CVP | hsa-miR-106b-star_st, hsa-miR-1181_st, hsa-miR-25-star_st, hsa-miR- |
| | 432_st, hsa-miR-551b-star_st, hsa-miR-652_st, hsa-miR-671-5p_st, hsa- |
| | miR-93-star_st |
| COPE | hsa-miR-766_st, hsa-miR-34b_st, ACA48_x_st, |
| | ENSG00000202498_x_st, HBII-85-26_st, HBII-85-6_x_st, hsa-miR-106b- |
| | star_st, hsa-miR-1181_st, hsa-miR-124_st, hsa-miR-1281_st, hsa-miR- |
| | 1299_st, hsa-miR-140-3p_st, hsa-miR-195-star_st, hsa-miR-25-star_st, |
| | hsa-miR-33b-star_st, hsa-miR-409-3p_st, hsa-miR-432_st, hsa-miR-551b- |
| | star_st, hsa-miR-631_st, hsa-miR-671-5p_st, hsa-miR-93-star_st |
| MBVP | hsa-miR-766_st, hsa-miR-34b_st, ACA10_s_st, ACA11_st, ACA13_st, |
| | ACA18_x_st, ACA21_st, ACA40_x_st, ACA41_x_st, ACA48_st, |
| | ACA48_x_st, ACA51_x_st, ACA57_st, ACA61_st, ACA7_s_st, ACA9_st, |
| | ENSG00000199411_s_st, ENSG00000200879_st, |
| | ENSG00000200932_st, ENSG00000201859_x_st, |
| | ENSG00000202252_st, ENSG00000207002_st, |
| | ENSG00000207002_x_st, HBII-115_st, HBII-135_x_st, HBII-180A_x_st, |
| | HBII-180C_x_st, HBII-202_st, HBII-239_st, HBII-336_st, HBII-429_st, |
| | HBII-55_st, HBII-85-26_st, HBII-85-6_x_st, U104_st, U13_st, U17a_st, |
| | U17a_x_st, U17b_st, U17b_x_st, U22_st, U25_st, U27_st, U29_st, U3- |
| | 2_s_st, U30_st, U31_x_st, U32A_x_st, U33_st, U34_st, U38A_st, |
| | U38B_st, U41_st, U48_st, U49A_st, U49A_x_st, U49B_s_st, U52_st, |
| | U55_st, U55_x_st, U56_st, U56_x_st, U57_st, U64_st, U67_st, U67_x_st, |
| | U68_st, U68_x_st, U71a_st, U71b_x_st, U71d_st, U71d_x_st, U74_x_st, |
| | U78_s_st, U78_x_st, U83B_st, U89_st, U8_x_st, U95_st, hsa-miR- |
| | 106a_st, hsa-miR-106b-star_st, hsa-miR-1183_st, hsa-miR-1246_st, hsa- |
| | miR-124_st, hsa-miR-1254_st, hsa-miR-1275_st, hsa-miR-1281_st, hsa- |
| | miR-1299_st, hsa-miR-140-3p_st, hsa-miR-142-3p_st, hsa-miR-142- |
| | 5p_st, hsa-miR-153_st, hsa-miR-17-star_st, hsa-miR-17_st, hsa-miR- |
| | 181a-star_st, hsa-miR-18a-star_st, hsa-miR-18a_st, hsa-miR-195-star_st, |
| | hsa-miR-19a_st, hsa-miR-19b_st, hsa-miR-20a_st, hsa-miR-223_st, hsa- |
| | miR-25-star_st, hsa-miR-297_st, hsa-miR-33b-star_st, hsa-miR-423- |
| | 3p_st, hsa-miR-423-5p_st, hsa-miR-491-3p_st, hsa-miR-595_st, hsa-miR- |
| | 631_st, hsa-miR-663b_st, hsa-miR-671-5p_st, hsa-miR-768-3p_st, hsa- |
| | miR-768-5p_st, hsa-miR-769-5p_st, hsa-miR-874_st, hsa-miR-877- |
| | star_st, hsa-miR-92a-1-star_st, hsa-miR-92a_st, hsa-miR-93-star_st |
| MTX | hsa-miR-766_st, hsa-miR-34b_st, ACA10_s_st, ACA18_x_st, |
| | ACA48_x_st, ACA51_x_st, ENSG00000199411_s_st, |
| | ENSG00000200879_st, HBII-180A_x_st, HBII-180C_x_st, HBII-202_st, |
| | HBII-429_st, HBII-55_st, U104_st, U17a_st, U17b_st, U17b_x_st, U25_st, |
| | U26_st, U27_st, U29_st, U3-2_s_st, U30_st, U31_x_st, U33_st, U38A_st, |
| | U48_st, U49A_st, U49A_x_st, U49B_s_st, U49B_x_st, U55_st, U55_x_st, |
| | U56_st, U56_x_st, U57_st, U67_st, U67_x_st, U74_x_st, U78_x_st, |
| | U89_st, hsa-miR-106a_st, hsa-miR-1246_st, hsa-miR-1254_st, hsa-miR- |
| | 1275_st, hsa-miR-17-star_st, hsa-miR-17_st, hsa-miR-18a-star_st, hsa- |
| | miR-18a_st, hsa-miR-18b_st, hsa-miR-19a_st, hsa-miR-19b_st, hsa-miR- |
| | 20a_st, hsa-miR-25-star_st, hsa-miR-297_st, hsa-miR-33b-star_st, hsa- |
| | miR-663b_st, hsa-miR-768-5p_st, hsa-miR-92a-1-star_st, hsa-miR- |
| | 92a_st, hsa-miR-936_st |
| DHAP | hsa-miR-766_st, hsa-miR-34b_st, ACA7_s_st, ENSG00000199282_st, |
| | ENSG00000200879_st, ENSG00000201299_x_st, |
| | ENSG00000201859_x_st, ENSG00000202252_st, |
| | ENSG00000202498_x_st, HBII-202_st, HBII-336_st, HBII-429_st, HBII- |
| | 438A_s_st, HBII-55_st, HBII-85-11_st, HBII-85-26_st, HBII-85-29_x_st, |
| | HBII-85-2_x_st, HBII-85-6_x_st, U104_st, U13_st, U17a_st, U17a_x_st, |
| | U17b_st, U17b_x_st, U3-2_s_st, U30_st, U33_st, U41_st, U48_st, |
| | U49A_st, U49A_x_st, U49B_s_st, U52_st, U55_st, U55_x_st, U57_st, |
| | U67_st, U71b_x_st, U78_s_st, U83_st, hsa-miR-106b-star_st, hsa-miR- |
| | 1183_st, hsa-miR-1207-5p_st, hsa-miR-1268_st, hsa-miR-1281_st, hsa- |
| | miR-140-3p_st, hsa-miR-150_st, hsa-miR-155_st, hsa-miR-181a-star_st, |
| | hsa-miR-181a_st, hsa-miR-181b_st, hsa-miR-181c_st, hsa-miR-195- |
| | star_st, hsa-miR-198_st, hsa-miR-20b-star_st, hsa-miR-223_st, hsa-miR- |
| | 297_st, hsa-miR-33b-star_st, hsa-miR-342-3p_st, hsa-miR-342-5p_st, |
| | hsa-miR-424-star_st, hsa-miR-432_st, hsa-miR-503_st, hsa-miR-574- |
| | 5p_st, hsa-miR-613_st, hsa-miR-615-3p_st, hsa-miR-631_st, hsa-miR- |
| | 768-5p_st, hsa-miR-877-star_st, hsa-miR-92a-2-star_st, hsa-miR-938_st |
| Prednisolone | hsa-miR-766_st, ACA18_x_st, ACA24_s_st, ACA24_x_st, ACA25_x_st, |
| | ACA3-2_st, ACA3_st, ACA40_x_st, ACA47_st, ACA48_st, ACA52_st, |
| | ACA54_st, ACA62_st, ACA7_s_st, ENSG00000199411_s_st, |
| | ENSG00000200394_st, ENSG00000200879_st, ENSG00000207002_st, |
| | HBII-180A_x_st, HBII-202_st, HBII-239_st, HBII-382_s_st, HBII-419_st, |
| | HBII-429_st, HBII-52-32_x_st, HBII-55_st, U104_st, U17a_st, U17b_st, |
| | U17b_x_st, U25_st, U26_st, U27_st, U28_st, U29_st, U3-2_s_st, U30_st, |
| | U31_st, U31_x_st, U33_st, U35A_st, U36A_x_st, U38A_st, U38B_st, |
| | U41_st, U46_st, U46_x_st, U49A_st, U50B_st, U50B_x_st, U50_st, |
| | U55_st, U56_st, U56_x_st, U57_st, U67_st, U67_x_st, U68_st, U68_x_st, |
| | U70_x_st, U73a_st, U78_s_st, U78_x_st, U83_st, U93_st, U95_st, hsa- |
| | miR-1181_st, hsa-miR-1207-5p_st, hsa-miR-1246_st, hsa-miR-1268_st, |
| | hsa-miR-1275_st, hsa-miR-17-star_st, hsa-miR-17_st, hsa-miR-195- |
| | star_st, hsa-miR-19b_st, hsa-miR-297_st, hsa-miR-571_st, hsa-miR-768- |
| | 3p_st, hsa-miR-768-5p_st, hsa-miR-877-star_st, hsa-miR-92a-1-star_st, |
| | hsa-miR-92a-2-star_st, hsa-miR-92a_st, hsa-miR-938_st, hsa-miR-939_st |
| Cyclophosp | ENSG00000200879_st, ENSG00000202252_st, HBII-142_st, HBII- |
| | 142_x_st, HBII-202_st, U42A_st, U59B_st, hsa-miR-1202_st, hsa-miR- |
| | 184_st, hsa-miR-187_st, hsa-miR-191_st, hsa-miR-196a_st, hsa-miR- |
| | 203_st, hsa-miR-29b-2-star_st, hsa-miR-29c-star_st, hsa-miR-328_st, |
| | hsa-miR-375_st, hsa-miR-423-3p_st, hsa-miR-423-5p_st, hsa-miR- |
| | 449a_st, hsa-miR-449b_st, hsa-miR-489_st, hsa-miR-768-3p_st |
| Doxorubicin | hsa-miR-766_st, hsa-miR-34b_st, ACA13_st, ACA48_x_st, U104_st, |
| | U55_st, U55_x_st, U74_x_st, hsa-miR-106a-star_st, hsa-miR-106b- |
| | star_st, hsa-miR-106b_st, hsa-miR-124_st, hsa-miR-1281_st, hsa-miR- |
| | 1299_st, hsa-miR-140-3p_st, hsa-miR-195-star_st, hsa-miR-297_st, hsa- |
| | miR-29b-2-star_st, hsa-miR-33b-star_st, hsa-miR-342-3p_st, hsa-miR- |
| | 342-5p_st, hsa-miR-432_st, hsa-miR-550-star_st, hsa-miR-629-star_st, |
| | hsa-miR-629_st, hsa-miR-652_st, hsa-miR-654-3p_st, hsa-miR-671- |
| | 5p_st, hsa-miR-768-3p_st, hsa-miR-877-star_st, hsa-miR-93-star_st, hsa- |
| | miR-93_st |
| Vincristine | hsa-miR-106b-star_st, hsa-miR-25-star_st, hsa-miR-432_st, hsa-miR- |
| | 551b-star_st, hsa-miR-671-5p_st, hsa-miR-93-star_st |
| ICE | hsa-miR-766_st, hsa-miR-34b_st, 14qII-14_st, 14qII-1_st, 14qII-26_st, |
| | ACA48_x_st, ENSG00000199282_st, ENSG00000201859_x_st, |
| | ENSG00000202498_x_st, HBII-85-26_st, HBII-85-6_x_st, U52_st, |
| | U55_st, U55_x_st, U8_x_st, hsa-miR-106b-star_st, hsa-miR-124_st, hsa- |
| | miR-127-3p_st, hsa-miR-1281_st, hsa-miR-1299_st, hsa-miR-140-3p_st, |
| | hsa-miR-181a-star_st, hsa-miR-181a_st, hsa-miR-181b_st, hsa-miR- |
| | 181c_st, hsa-miR-181d_st, hsa-miR-195-star_st, hsa-miR-199b-3p_st, |
| | hsa-miR-297_st, hsa-miR-299-3p_st, hsa-miR-342-3p_st, hsa-miR-342- |
| | 5p_st, hsa-miR-377-star_st, hsa-miR-409-3p_st, hsa-miR-409-5p_st, hsa- |
| | miR-410_st, hsa-miR-432_st, hsa-miR-487b_st, hsa-miR-615-3p_st, hsa- |
| | miR-631_st, hsa-miR-654-3p_st, hsa-miR-671-5p_st, hsa-miR-92b_st, |
| | hsa-miR-93-star_st, hsa-miR-938_st |
| VIM | hsa-miR-766_st, hsa-miR-34b_st, ACA10_s_st, ACA11_st, ACA13_st, |
| | ACA18_x_st, ACA21_st, ACA48_st, ACA48_x_st, ACA7_s_st, |
| | ENSG00000199411_s_st, ENSG00000200879_st, |
| | ENSG00000200932_st, ENSG00000201859_x_st, |
| | ENSG00000202252_st, ENSG00000202498_x_st, |
| | ENSG00000207002_st, HBII-115_st, HBII-180A_x_st, HBII-202_st, HBII- |
| | 336_st, HBII-429_st, HBII-55_st, HBII-85-26_st, HBII-85-6_x_st, U104_st, |
| | U13_st, U17a_st, U17b_st, U17b_x_st, U25_st, U27_st, U29_st, U3- |
| | 2_s_st, U30_st, U31_x_st, U32A_x_st, U33_st, U34_st, U38A_st, U41_st, |
| | U48_st, U49A_st, U49A_x_st, U49B_s_st, U52_st, U55_st, U55_x_st, |
| | U56_st, U56_x_st, U57_st, U64_st, U67_st, U67_x_st, U68_st, U68_x_st, |
| | U71b_x_st, U71d_x_st, U74_x_st, U83B_st, U8_x_st, hsa-miR-106a_st, |
| | hsa-miR-106b-star_st, hsa-miR-1246_st, hsa-miR-124_st, hsa-miR- |
| | 1254_st, hsa-miR-1275_st, hsa-miR-1281_st, hsa-miR-1307_st, hsa-miR- |
| | 153_st, hsa-miR-17-star_st, hsa-miR-17_st, hsa-miR-181a-star_st, hsa- |
| | miR-18a-star_st, hsa-miR-18a_st, hsa-miR-195-star_st, hsa-miR-19a_st, |
| | hsa-miR-19b_st, hsa-miR-223_st, hsa-miR-25-star_st, hsa-miR-297_st, |
| | hsa-miR-33b-star_st, hsa-miR-423-3p_st, hsa-miR-423-5p_st, hsa-miR- |
| | 663b_st, hsa-miR-768-3p_st, hsa-miR-768-5p_st, hsa-miR-769-5p_st, |
| | hsa-miR-92a-1-star_st, hsa-miR-92a_st, hsa-miR-93-star_st |
| MIME | hsa-miR-766_st, hsa-miR-34b_st, ACA10_s_st, ACA11_st, ACA13_st, |
| | ACA18_x_st, ACA21_st, ACA40_x_st, ACA48_x_st, ACA51_x_st, |
| | ACA55_st, ACA61_st, ACA7_s_st, ACA9_st, ENSG00000199282_st, |
| | ENSG00000199411_s_st, ENSG00000200879_st, |
| | ENSG00000202252_st, ENSG00000202498_x_st, |
| | ENSG00000207002_st, ENSG00000207002_x_st, HBII-180A_x_st, HBII- |
| | 180C_x_st, HBII-202_st, HBII-239_st, HBII-336_st, HBII-429_st, HBII- |
| | 55_st, HBII-85-26_st, HBII-85-6_x_st, HBII-85-8_x_st, U104_st, U13_st, |
| | U17a_st, U17a_x_st, U17b_st, U17b_x_st, U22_st, U25_st, U26_st, |
| | U27_st, U28_st, U29_st, U3-2_s_st, U30_st, U31_x_st, U33_st, U34_st, |
| | U36A_st, U38A_st, U41_st, U43_x_st, U48_st, U49A_st, U49A_x_st, |
| | U49B_s_st, U52_st, U55_st, U55_x_st, U56_st, U56_x_st, U57_st, |
| | U59B_st, U64_st, U67_st, U68_st, U71a_st, U71b_x_st, U71d_x_st, |
| | U74_x_st, U75_st, U78_s_st, U78_x_st, U83B_st, U83_st, hsa-miR- |
| | 106a_st, hsa-miR-106b-star_st, hsa-miR-1183_st, hsa-miR-1246_st, hsa- |
| | miR-124_st, hsa-miR-1254_st, hsa-miR-1275_st, hsa-miR-1281_st, hsa- |
| | miR-1307_st, hsa-miR-153_st, hsa-miR-17-star_st, hsa-miR-17_st, hsa- |
| | miR-181a-star_st, hsa-miR-181b_st, hsa-miR-18a-star_st, hsa-miR- |
| | 18a_st, hsa-miR-18b_st, hsa-miR-195-star_st, hsa-miR-19a_st, hsa-miR- |
| | 19b_st, hsa-miR-20a_st, hsa-miR-223_st, hsa-miR-25-star_st, hsa-miR- |
| | 297_st, hsa-miR-33b-star_st, hsa-miR-423-3p_st, hsa-miR-423-5p_st, |
| | hsa-miR-425-star_st, hsa-miR-595_st, hsa-miR-615-3p_st, hsa-miR- |
| | 663b_st, hsa-miR-768-3p_st, hsa-miR-768-5p_st, hsa-miR-769-5p_st, |
| | hsa-miR-92a-1-star_st, hsa-miR-92a_st, hsa-miR-93-star_st |
| Bendamustine | hsa-miR-766_st, hsa-miR-34b_st, ACA11_st, ACA24_x_st, ACA7_s_st, |
| | HBII-115_st, HBII-438A_s_st, HBII-85-11_st, U13_st, U49B_s_st, hsa- |
| | miR-106b-star_st, hsa-miR-128_st, hsa-miR-1299_st, hsa-miR-142-5p_st, |
| | hsa-miR-153_st, hsa-miR-155_st, hsa-miR-15a-star_st, hsa-miR-15a_st, |
| | hsa-miR-181a-star_st, hsa-miR-181a_st, hsa-miR-181b_st, hsa-miR- |
| | 181c_st, hsa-miR-20b-star_st, hsa-miR-29b-2-star_st, hsa-miR-29c- |
| | star_st, hsa-miR-29c_st, hsa-miR-342-5p_st, hsa-miR-361-5p_st, hsa- |
| | miR-363-star_st, hsa-miR-647_st, hsa-miR-93-star_st |
| ESHAP | hsa-miR-766_st, hsa-miR-34b_st, ACA48_x_st, ACA7_s_st, |
| | ENSG00000201859_x_st, HBII-202_st, HBII-429_st, HBII-438A_s_st, |
| | HBII-55_st, HBII-85-11_st, HBII-85-26_st, HBII-85-2_x_st, HBII-85-6_x_st, |
| | HBII-85-8_x_st, U104_st, U17b_st, U17b_x_st, U33_st, U48_st, U52_st, |
| | U55_st, U55_x_st, U57_st, U67_st, U74_x_st, U78_s_st, U78_x_st, |
| | U95_st, hsa-miR-124_st, hsa-miR-1281_st, hsa-miR-140-3p_st, hsa-miR- |
| | 181a-star_st, hsa-miR-181a_st, hsa-miR-181b_st, hsa-miR-181c_st, hsa- |
| | miR-195-star_st, hsa-miR-223_st, hsa-miR-297_st, hsa-miR-342-3p_st, |
| | hsa-miR-342-5p_st, hsa-miR-424-star_st, hsa-miR-768-5p_st |
| ABVD | hsa-miR-766_st, 14qII-14_st, 14qII-14_x_st, 14qII-1_st, 14qII-1_x_st, |
| | 14qII-22_x_st, 14qII-26_st, 14qII-26_x_st, HBII-85-26_st, HBII-85-6_x_st, |
| | U22_st, U48_st, U55_st, U55_x_st, hsa-miR-124_st, hsa-miR-125b-1- |
| | star_st, hsa-miR-127-3p_st, hsa-miR-1271_st, hsa-miR-1281_st, hsa- |
| | miR-1299_st, hsa-miR-134_st, hsa-miR-140-3p_st, hsa-miR-143_st, hsa- |
| | miR-145_st, hsa-miR-154_st, hsa-miR-195-star_st, hsa-miR-199a-5p_st, |
| | hsa-miR-199b-3p_st, hsa-miR-299-3p_st, hsa-miR-342-3p_st, hsa-miR- |
| | 370_st, hsa-miR-376c_st, hsa-miR-377-star_st, hsa-miR-379_st, hsa- |
| | miR-381_st, hsa-miR-382_st, hsa-miR-409-3p_st, hsa-miR-409-5p_st, |
| | hsa-miR-411_st, hsa-miR-431_st, hsa-miR-432_st, hsa-miR-433_st, hsa- |
| | miR-485-3p_st, hsa-miR-485-5p_st, hsa-miR-487a_st, hsa-miR-487b_st, |
| | hsa-miR-493_st, hsa-miR-495_st, hsa-miR-629-star_st, hsa-miR-654- |
| | 3p_st, hsa-miR-665_st, hsa-miR-671-5p_st, hsa-miR-758_st, hsa-miR- |
| | 923_st |
| BEACOPP | 14qII-14_st, 14qII-14_x_st, 14qII-1_st, 14qII-1_x_st, 14qII-22_x_st, 14qII- |
| | 26_st, 14qII-26_x_st, 14qII-3_st, ENSG00000202498_x_st, HBII-85- |
| | 26_st, HBII-85-6_x_st, hsa-miR-106b-star_st, hsa-miR-124_st, hsa-miR- |
| | 127-3p_st, hsa-miR-127-5p_st, hsa-miR-1281_st, hsa-miR-1299_st, hsa- |
| | miR-134_st, hsa-miR-140-3p_st, hsa-miR-154_st, hsa-miR-181a-star_st, |
| | hsa-miR-195-star_st, hsa-miR-199a-5p_st, hsa-miR-214-star_st, hsa-miR- |
| | 299-3p_st, hsa-miR-337-5p_st, hsa-miR-339-5p_st, hsa-miR-342-3p_st, |
| | hsa-miR-370_st, hsa-miR-376c_st, hsa-miR-377-star_st, hsa-miR-379_st, |
| | hsa-miR-381_st, hsa-miR-382_st, hsa-miR-409-3p_st, hsa-miR-409- |
| | 5p_st, hsa-miR-410_st, hsa-miR-411_st, hsa-miR-431_st, hsa-miR- |
| | 432_st, hsa-miR-433_st, hsa-miR-485-3p_st, hsa-miR-485-5p_st, hsa- |
| | miR-487a_st, hsa-miR-487b_st, hsa-miR-493_st, hsa-miR-615-3p_st, |
| | hsa-miR-654-3p_st, hsa-miR-665_st, hsa-miR-671-5p_st, hsa-miR-768- |
| | 3p_st, hsa-miR-92b_st |
| CBV | hsa-miR-766_st, hsa-miR-34b_st, ACA48_x_st, ACA7_s_st, |
| | ENSG00000199282_st, ENSG00000202252_st, |
| | ENSG00000202498_x_st, ENSG00000207002_st, HBII-202_st, HBII-85- |
| | 26_st, HBII-85-6_x_st, U104_st, U17a_st, U34_st, U41_st, U52_st, |
| | U55_st, U55_x_st, U57_st, U67_st, U74_x_st, hsa-miR-106a-star_st, hsa- |
| | miR-106b-star_st, hsa-miR-1181_st, hsa-miR-1183_st, hsa-miR-1228_st, |
| | hsa-miR-124_st, hsa-miR-1281_st, hsa-miR-1299_st, hsa-miR-140-3p_st, |
| | hsa-miR-181a-star_st, hsa-miR-181a_st, hsa-miR-181b_st, hsa-miR- |
| | 181c_st, hsa-miR-195-star_st, hsa-miR-223_st, hsa-miR-297_st, hsa- |
| | miR-339-5p_st, hsa-miR-342-3p_st, hsa-miR-342-5p_st, hsa-miR-432_st, |
| | hsa-miR-551b-star_st, hsa-miR-610_st, hsa-miR-615-3p_st, hsa-miR- |
| | 631_st, hsa-miR-671-5p_st, hsa-miR-768-3p_st, hsa-miR-768-5p_st, hsa- |
| | miR-769-5p_st, hsa-miR-92b_st, hsa-miR-93-star_st, hsa-miR-938_st |
| CEPP | 14qII-12_st, 14qII-14_st, 14qII-14_x_st, 14qII-1_st, 14qII-1_x_st, 14qII- |
| | 22_x_st, 14qII-26_st, 14qII-26_x_st, 14qII-3_st, ENSG00000202498_x_st, |
| | HBII-336_st, HBII-85-26_st, HBII-85-6_x_st, hsa-miR-125b-1-star_st, hsa- |
| | miR-127-3p_st, hsa-miR-127-5p_st, hsa-miR-1271_st, hsa-miR-1281_st, |
| | hsa-miR-134_st, hsa-miR-140-3p_st, hsa-miR-143-star_st, hsa-miR- |
| | 143_st, hsa-miR-145_st, hsa-miR-154_st, hsa-miR-155_st, hsa-miR- |
| | 181a-star_st, hsa-miR-181a_st, hsa-miR-181b_st, hsa-miR-181c_st, hsa- |
| | miR-199a-5p_st, hsa-miR-199b-3p_st, hsa-miR-21-star_st, hsa-miR-214- |
| | star_st, hsa-miR-27a_st, hsa-miR-299-3p_st, hsa-miR-337-5p_st, hsa- |
| | miR-339-5p_st, hsa-miR-342-3p_st, hsa-miR-34c-3p_st, hsa-miR-370_st, |
| | hsa-miR-376c_st, hsa-miR-377-star_st, hsa-miR-379_st, hsa-miR-381_st, |
| | hsa-miR-382_st, hsa-miR-409-3p_st, hsa-miR-409-5p_st, hsa-miR- |
| | 411_st, hsa-miR-431_st, hsa-miR-432_st, hsa-miR-433_st, hsa-miR-485- |
| | 3p_st, hsa-miR-485-5p_st, hsa-miR-487a_st, hsa-miR-487b_st, hsa-miR- |
| | 493_st, hsa-miR-494_st, hsa-miR-543_st, hsa-miR-574-3p_st, hsa-miR- |
| | 615-3p_st, hsa-miR-654-3p_st, hsa-miR-665_st, hsa-miR-671-5p_st, hsa- |
| | miR-758_st, hsa-miR-92b_st |
| COPP | hsa-miR-106b-star_st, hsa-miR-1181_st, hsa-miR-432_st, hsa-miR-551b- |
| | star_st, hsa-miR-652_st, hsa-miR-671-5p_st, hsa-miR-93-star_st, hsa- |
| | miR-93_st |
| EPOCH | hsa-miR-766_st, hsa-miR-34b_st, ACA48_x_st, |
| | ENSG00000202498_x_st, HBII-85-26_st, HBII-85-6_x_st, U55_st, |
| | U55_x_st, hsa-miR-106b-star_st, hsa-miR-106b_st, hsa-miR-1181_st, |
| | hsa-miR-124_st, hsa-miR-127-3p_st, hsa-miR-1281_st, hsa-miR-1299_st, |
| | hsa-miR-140-3p_st, hsa-miR-195-star_st, hsa-miR-25-star_st, hsa-miR- |
| | 33b-star_st, hsa-miR-409-3p_st, hsa-miR-432_st, hsa-miR-551b-star_st, |
| | hsa-miR-629-star_st, hsa-miR-652_st, hsa-miR-654-3p_st, hsa-miR-671- |
| | 5p_st, hsa-miR-877-star_st, hsa-miR-92b_st, hsa-miR-93-star_st, hsa- |
| | miR-93_st |
| FCM | hsa-miR-766_st, hsa-miR-34b_st, ACA7_s_st, HBII-438A_s_st, HBII-85- |
| | 11_st, HBII-85-2_x_st, HBII-85-6_x_st, U104_st, U52_st, hsa-miR- |
| | 1281_st, hsa-miR-1299_st, hsa-miR-140-3p_st, hsa-miR-155_st, hsa- |
| | miR-181a-2-star_st, hsa-miR-181a-star_st, hsa-miR-181a_st, hsa-miR- |
| | 181b_st, hsa-miR-181c_st, hsa-miR-181d_st, hsa-miR-223_st, hsa-miR- |
| | 342-3p_st, hsa-miR-342-5p_st, hsa-miR-34c-3p_st, hsa-miR-424-star_st, |
| | hsa-miR-489_st, hsa-miR-503_st, hsa-miR-768-5p_st, hsa-miR-874_st, |
| | hsa-miR-92b_st |
| GemOx | hsa-miR-34b_st, hsa-miR-126_st, hsa-miR-153_st, hsa-miR-19b_st, hsa- |
| | miR-424-star_st, hsa-miR-491-3p_st, hsa-miR-503_st |
| CVAD | hsa-miR-766_st, hsa-miR-34b_st, ENSG00000201125_x_st, |
| Course A | ENSG00000201859_x_st, ENSG00000202498_x_st, HBII-85-26_st, HBII- |
| | 85-6_x_st, U17b_st, U17b_x_st, U48_st, U49A_st, U49A_x_st, |
| | U49B_s_st, U55_st, U55_x_st, hsa-miR-106a-star_st, hsa-miR-106b- |
| | star_st, hsa-miR-1183_st, hsa-miR-1207-5p_st, hsa-miR-1271_st, hsa- |
| | miR-1281_st, hsa-miR-128_st, hsa-miR-150_st, hsa-miR-181c-star_st, |
| | hsa-miR-195-star_st, hsa-miR-20b-star_st, hsa-miR-223_st, hsa-miR- |
| | 297_st, hsa-miR-324-3p_st, hsa-miR-33b-star_st, hsa-miR-342-3p_st, |
| | hsa-miR-342-5p_st, hsa-miR-361-5p_st, hsa-miR-424-star_st, hsa-miR- |
| | 424_st, hsa-miR-432_st, hsa-miR-503_st, hsa-miR-631_st, hsa-miR-671- |
| | 5p_st, hsa-miR-769-5p_st, hsa-miR-92a-2-star_st |
| CVAD | hsa-miR-766_st, hsa-miR-34b_st, ACA21_st, ACA48_st, ACA48_x_st, |
| Course B | ENSG00000200879_st, HBII-202_st, HBII-336_st, HBII-429_st, HBII- |
| | 438A_s_st, HBII-55_st, U104_st, U17a_x_st, U17b_st, U17b_x_st, |
| | U25_st, U27_st, U29_st, U30_st, U31_x_st, U32A_x_st, U33_st, U34_st, |
| | U48_st, U49A_st, U49A_x_st, U49B_s_st, U55_st, U55_x_st, U56_st, |
| | U56_x_st, U57_st, U67_st, U74_x_st, U78_s_st, U78_x_st, hsa-miR- |
| | 153_st, hsa-miR-17_st, hsa-miR-181a-star_st, hsa-miR-18a-star_st, hsa- |
| | miR-18a_st, hsa-miR-223_st, hsa-miR-25-star_st, hsa-miR-297_st, hsa- |
| | miR-33b-star_st, hsa-miR-663b_st, hsa-miR-768-5p_st, hsa-miR-92a_st |
| MINE | hsa-miR-766_st, hsa-miR-34b_st, 14qII-14_st, 14qII-14_x_st, 14qII-1_st, |
| | 14qII-26_st, 14qII-26_x_st, ENSG00000202498_x_st, HBII-85-26_st, |
| | HBII-85-6_x_st, U104_st, U52_st, U55_st, U55_x_st, hsa-miR-124_st, |
| | hsa-miR-127-3p_st, hsa-miR-1281_st, hsa-miR-1299_st, hsa-miR-140- |
| | 3p_st, hsa-miR-181a-star_st, hsa-miR-181a_st, hsa-miR-181b_st, hsa- |
| | miR-181c-star_st, hsa-miR-181c_st, hsa-miR-181d_st, hsa-miR-195- |
| | star_st, hsa-miR-297_st, hsa-miR-299-3p_st, hsa-miR-29b-2-star_st, hsa- |
| | miR-339-5p_st, hsa-miR-33b-star_st, hsa-miR-342-3p_st, hsa-miR-342- |
| | 5p_st, hsa-miR-377-star_st, hsa-miR-379_st, hsa-miR-381_st, hsa-miR- |
| | 409-3p_st, hsa-miR-409-5p_st, hsa-miR-411_st, hsa-miR-432_st, hsa- |
| | miR-433_st, hsa-miR-487b_st, hsa-miR-493_st, hsa-miR-654-3p_st, hsa- |
| | miR-671-5p_st, hsa-miR-768-3p_st, hsa-miR-768-5p_st, hsa-miR-92b_st, |
| | hsa-miR-938_st |
| MOPP | hsa-miR-766_st, hsa-miR-34b_st, ACA13_st, ACA21_st, ACA48_x_st, |
| | ENSG00000199282_st, ENSG00000201299_x_st, HBII-239_st, HBII- |
| | 336_st, HBII-85-26_st, HBII-85-2_x_st, HBII-85-6_x_st, U104_st, U13_st, |
| | U22_st, U55_st, U55_x_st, U74_x_st, U8_x_st, hsa-miR-124_st, hsa-miR- |
| | 1281_st, hsa-miR-1299_st, hsa-miR-140-3p_st, hsa-miR-181a-star_st, |
| | hsa-miR-181a_st, hsa-miR-181b_st, hsa-miR-181c_st, hsa-miR-195- |
| | star_st, hsa-miR-297_st, hsa-miR-29b-2-star_st, hsa-miR-331-5p_st, hsa- |
| | miR-33b-star_st, hsa-miR-342-3p_st, hsa-miR-342-5p_st, hsa-miR-424- |
| | star_st, hsa-miR-432_st, hsa-miR-489_st, hsa-miR-768-3p_st, hsa-miR- |
| | 768-5p_st, hsa-miR-874_st, hsa-miR-877-star_st, hsa-miR-92b_st |
| Romidepsin | hsa-miR-105_st, hsa-miR-106b_st, hsa-miR-185_st, hsa-miR-188-5p_st, |
| | hsa-miR-25_st, hsa-miR-320c_st, hsa-miR-362-5p_st, hsa-miR-500- |
| | star_st, hsa-miR-500_st, hsa-miR-501-3p_st, hsa-miR-501-5p_st, hsa- |
| | miR-502-3p_st, hsa-miR-532-3p_st, hsa-miR-532-5p_st, hsa-miR-584_st, |
| | hsa-miR-611_st, hsa-miR-652_st, hsa-miR-660_st, hsa-miR-767-5p_st, |
| | hsa-miR-769-3p_st, hsa-miR-877_st, hsa-miR-93_st |
| CHOP | hsa-miR-766_st, ACA48_x_st, U55_x_st, hsa-miR-106b-star_st, hsa-miR- |
| | 106b_st, hsa-miR-1181_st, hsa-miR-124_st, hsa-miR-1299_st, hsa-miR- |
| | 25-star_st, hsa-miR-33b-star_st, hsa-miR-432_st, hsa-miR-551b-star_st, |
| | hsa-miR-629-star_st, hsa-miR-629_st, hsa-miR-652_st, hsa-miR-654- |
| | 3p_st, hsa-miR-671-5p_st, hsa-miR-877-star_st, hsa-miR-93-star_st, hsa- |
| | miR-93_st |
| CHOEP | hsa-miR-766_st, hsa-miR-34b_st, ACA48_x_st, |
| | ENSG00000202498_x_st, HBII-85-26_st, HBII-85-6_x_st, U55_st, |
| | U55_x_st, hsa-miR-106b-star_st, hsa-miR-106b_st, hsa-miR-1181_st, |
| | hsa-miR-124_st, hsa-miR-127-3p_st, hsa-miR-1281_st, hsa-miR-1299_st, |
| | hsa-miR-140-3p_st, hsa-miR-195-star_st, hsa-miR-25-star_st, hsa-miR- |
| | 33b-star_st, hsa-miR-409-3p_st, hsa-miR-432_st, hsa-miR-551b-star_st, |
| | hsa-miR-629-star_st, hsa-miR-652_st, hsa-miR-654-3p_st, hsa-miR-671- |
| | 5p_st, hsa-miR-877-star_st, hsa-miR-92b_st, hsa-miR-93-star_st, hsa- |
| | miR-93_st |
| BelCHOP | hsa-miR-766_st, hsa-miR-34b_st, ACA10_s_st, ACA13_st, ACA18_x_st, |
| | ACA48_x_st, ACA9_st, ENSG00000200932_st, HBII-180A_x_st, HBII- |
| | 55_st, HBII-85-26_st, HBII-85-6_x_st, U104_st, U13_st, U17a_st, |
| | U17b_st, U17b_x_st, U22_st, U38A_st, U49B_s_st, U51_st, U55_st, |
| | U55_x_st, U56_st, U56_x_st, U57_st, U67_st, U71a_st, hsa-miR-106a_st, |
| | hsa-miR-106b-star_st, hsa-miR-106b_st, hsa-miR-1181_st, hsa-miR- |
| | 1228_st, hsa-miR-1246_st, hsa-miR-124_st, hsa-miR-1254_st, hsa-miR- |
| | 1268_st, hsa-miR-1281_st, hsa-miR-1299_st, hsa-miR-17-star_st, hsa- |
| | miR-181c-star_st, hsa-miR-18a-star_st, hsa-miR-18a_st, hsa-miR-18b_st, |
| | hsa-miR-195-star_st, hsa-miR-19a_st, hsa-miR-19b_st, hsa-miR-25- |
| | star_st, hsa-miR-25_st, hsa-miR-297_st, hsa-miR-33b-star_st, hsa-miR- |
| | 346_st, hsa-miR-551b-star_st, hsa-miR-593-star_st, hsa-miR-595_st, hsa- |
| | miR-611_st, hsa-miR-629-star_st, hsa-miR-629_st, hsa-miR-631_st, hsa- |
| | miR-652_st, hsa-miR-671-5p_st, hsa-miR-769-5p_st, hsa-miR-877- |
| | star_st, hsa-miR-93-star_st, hsa-miR-93_st |

Abbreviations used for specific treatment combinations are as follows: CVP = cyclophosphamide, vincristine; COPE = cyclophosphamide, vincristine, etoposide; MBVP = methotrexate, carmustine, teniposide; MTX = methotrexate; DHAP = dexamethasone, ara-C, cisplatin; ICE = Ifosfamide, carboplatin, etoposide; VIM = etoposide, ifosfamide, methotrexate; ESHAP = etoposide, cytarabine, methylprednisolone, and cisplatin; MIME = methyl-GAG, ifosfamide, methotrexate, etoposide; ABVD = adriamycin, bleomycin, vinblastine, dacarbazine; BEACOPP = bleomycin, etoposide, adriamycin, cyclophosphamide, vincristine, procarbazine; CBV = cyclophosphamide, carmustine (BCNU), etoposide; COPP = cyclophosphamide, vincristine, procarbazine; EPOCH = cyclophosphamide, etoposide, vincristine, doxorubicine; FCM = fludarabine, cyclophosphamide, mitoxantrone; GemOx = gemcitabine, oxaliplatin; CVAD-A = cyclophosphamide, vincristine, doxorubicin, dexamethasone; CVAD-B = methotrexate and cytarabine, MINE = ifosfamide, mitoxantrone, etoposide, MOPP = mechlorethamine, vincristine, procarbazine, CHOP = cyclophosphamide, vincristine, adriamycine, CHOEP = cyclophosphamide, vincristine, adriamycine, and etoposide, and BelCHOP = cyclophosphamide, vincristine, adriamycine, and belinostat.

Pre-clinical and clinical validation of such treatment response predictors have been developed by the present inventor for a number of other drug treatments (see, e.g., U.S. Pat. No. 8,445,198; PCT Publication No. WO2011/135459; PCT Publication No. WO2012163541; Chen et al., Mol. Cancer Ther. 11(1): 34-44, 2012; Bullinger et al., 2013, presented at ESMO conference 2012; Wang et al., J. National Cancer Inst. 105(17): 1284-91, 2013; Knudsen et al., PLOS ONE, 9(2): e87415, 2014; each of which is incorporated herein in its entirety).

The invention further features a method for predicting the responsiveness of a patient with DLBCL to a diversity of treatments (e.g., combination treatment) by assaying a level of expression of at least one biomarker (e.g., one, two, three, four, five, ten, twenty, or all biomarkers for each respective therapy shown in Table 1) in a biological sample from the patient, e.g., using a device, such as an array (e.g., a microarray). Methods of the invention featuring a device may include an array (e.g., a microarray) with one or more single-stranded oligonucleotide probes that have substantial identity (e.g., at least 85%, 90%, 95%, 99%, or 100% sequence identity) to a target nucleic acid molecule having a sequence that is complementary or identical to the nucleic acid sequence of one or more biomarkers described herein. For example, the probes can be used to detect one or more (e.g., two, three, four, five, ten, twenty, or all) of the biomarkers listed in Table 1, such as hsa-miR-766_st and hsa-miR-34b_st, or one or more biomarkers corresponding to a specific monotherapy or combination therapy shown in Table 1. As such, the methods and devices described herein can be used to, e.g., determine a patient's responsive to one or more cancer treatments (e.g., monotherapies or combination therapies) for DLBCL.

### Methods for Predicting a Patient's Responsiveness to Combination Treatment

The invention features diagnostic methods useful for the detection and screening of lymphoma patients (e.g., patients with DLBCL) that may be responsive to cancer treatments (e.g., combination therapies) using certain biomarkers, e.g., hsa-miR-766_st and hsa-miR-34b_st alone or in combination with one or more of the biomarkers shown in Table 1 for the specified treatment. The methods of the invention may be used for predicting a patient's responsiveness to treatment (e.g., one or more treatments shown in Table 1), and optionally, treating the cancer patient throughout the stages of DLBCL and/or in cases of disease relapse (e.g., after a first line treatment, a second line treatment, and a third line treatment). The methods can be performed using a biological sample from the patient. The biological sample can include, for example, cells, tissue (e.g., a tissue sample obtained from a tumor biopsy), blood, or serum. In certain embodiments, the biological sample is fresh frozen or formalin-fixed paraffin embedded (FFPE) tissue obtained from the subject, such as a tumor sample (e.g., a biopsy) from the tissue of interest (e.g., lymph nodes, thymus, spleen, and bone marrow).

Numerous methods of determining changes in the gene expression (e.g., mRNA or protein expression level) of the biomarkers of the invention (e.g., those listed in Table 1) are known in the art. Examples of sequencing based technologies include, but are not limited to, PCR, an array, a massarray, a beadarray, a microarray, a genechip, pyrosequencing, nanopore sequencing, sequencing by synthesis, sequencing by expansion, single molecule real time technology, true single molecule sequencing technology, sequencing by ligation, microfluidics, infrared fluorescence, next generation sequencing, and combinations thereof. Such techniques are not limited to ribonucleic acids (e.g., mRNA transcripts), but may also include amino acids using proteomic techniques (e.g., mass spectrometry or protein arrays). In certain embodiments, primers and probes comprising the sequences described herein are used to detect changes in the biomarkers (e.g., Table 2). Such changes in the nucleic acid sequence may include, but are not limited to, an increase or decrease in gene expression of one or more of the biomarkers (e.g., one or more of the biomarkers of Table 1) relative to a control sample or to other biomarkers. In specific embodiments, the biomarkers of the invention may include, but are not limited to, hsa-miR-766_st and/or hsa-miR-34b_st (e.g., the expression level of at least a second biomarker from among the biomarkers listed in Table 1 for the corresponding treatment may be assessed).

TABLE 2
Target sequence for each of the biomarkers listed
in Table 1. The target sequence is either a microRNA
or small nuclear RNA. The array (e.g., the
microarray, such as an Affymetrix array) described
in the methods of the invention may contain several
nucleotide probes for each target sequence.
Each probe is complementary to the target sequence.

| SEQ | | |
|---|---|---|
| ID NO | Nucleotide Probe | Target Sequence |
| 1 | 14qII-12_st | TGGACCAATGATGACAAATACCGGCGTATGAGTCTTGGA |
| | | TGATGAATAATACGTGTCTGGAACTCTGAGGTCCA |
| 2 | 14qII-14_st | TGGACCAATGATGACAACTGCCGGCGTATGAGTGTTGGG |
| | | TGATGAATAATACGTGTCTAGAACTCTGAGGTCCA |
| 3 | 14qII-14_x_st | TGGACCAATGATGACAACTGCCGGCGTATGAGTGTTGGG |
| | | TGATGAATAATACGTGTCTAGAACTCTGAGGTCCA |
| 4 | 14qII-1_st | TGGACCTATGATGATGACTGGTGGCGTATGAGTCATTGA |
| | | CGGTGAATACAGGTCTGGAAGTCTGAGGTCCA |
| 5 | 14qII-1_x_st | TGGACCTATGATGATGACTGGTGGCGTATGAGTCATTGA |
| | | CGGTGAATACAGGTCTGGAAGTCTGAGGTCCA |
| 6 | 14qII-22_x_st | TGGATCGATGATGACTACCGGTGGCGTATGAGTCATATG |
| | | TGATGAATACGTGTTTGGAACTCTGAGGTCCA |
| 7 | 14qII-26_st | TGGATCGATGATGAGCACTGGTGGAGTATGAGTCACATA |
| | | CGATGAATACGTGTCTGGAACTCTGAGGTCCA |
| 8 | 14qII-26_x_st | TGGATCGATGATGAGCACTGGTGGAGTATGAGTCACATA |
| | | CGATGAATACGTGTCTGGAACTCTGAGGTCCA |
| 9 | 14qII-3_st | TGGACCAATGATGACCACTGGTGGCGTTTGAGTCATGGA |
| | | CGATGAATACTACGTGTCTGAAACTCTGAGGTCCA |
| 10 | ACA10_s_st | GGTCTCTCAGCTCCGCTTAACCACACGGGTCCAGTGTGT |
| | | GCTTGGCGTGTTTTCAGGGAGGCAGAGAAAGGCTCTCCT |
| | | AATGCACGACAGACCCGCCCAGAATGGCCTCTCTGTTCC |
| | | TAGGAGTGCGACAATT |
| 11 | ACA11_st | GCGCTCACTAAGGCTCGGTCCTCTCCACGTGGTCCTGAC |
| | | CTGTCCTCTGTGAGCAAGAGAAACAGGACTGGTTTGGGG |
| | | GTGTCCTGTCTCAGTGGACACAGGACACCACGGTTTTCA |
| | | GTACAACA |
| 12 | ACA13_st | AGCCTTTGTGTTGCCCATTCACTTTGGAAACTAGTGAATG |
| | | TGGTGTCAAAAAAGGCGTAAATTAAACGCTTTGCAGCCTT |
| | | TTCCTGCCCTTAAATTTGATACCTTTGGTGTAGGAGCTGC |
| | | ATAAGTAACAGTT |
| 13 | ACA18_x_st | GTTGAGGTCTATCCCGATGGGGCTTTTCCTGTAGCCTGC |
| | | ACATCGTTGGAAACGCCTCATAGAGTAACTCTGTGGTTTT |
| | | ACTTTACTCACAGGACTATTGTTAGATCTGTGGGAAGGAA |
| | | TTACAAGACAGTT |
| 14 | ACA21_st | CCCCCTTTTAAAAGCACTCAATGGGCCTGTGGCTAATGA |
| | | CCTATTGAGCCGTCAAGAAAGGGGAGAGTGAAAACATCG |
| | | CTTTTGGGTGAAGTGGCAACATGTGTTGTTTGCTTCAATC |
| | | GGTGGTGTGACAAGG |
| 15 | ACA24_x_st | CTCCATGTATCTTTGGGACCTGTCAGCCGTGGCAGTCTC |
| | | CCTTCCTAGCCATGGAAGAGCATATCCTTGTTTATTGGCA |
| | | AAGCTGTCACCATTTAATTGGTATCAGATTCTGACTTGCA |
| | | CAAGTAACATTC |
| 16 | ACA40_x_st | TGCACTTATGTATGTTTTTGTTTAACGTGGACAAAGACTT |
| | | ACAGATAGGTGCAAAAAATAAATCCTCTTTTGCAACCCAG |
| | | AACTCATTGTTCAGTATGAGTTTTGATACATATAAGAAGG |
| | | GATATTA |
| 17 | ACA41_x_st | TTCCACAGCTACTGGTCTGCAGCTGTTCTTATGGTAGCA |
| | | GTTGTGGCATTCCTCTGTGGGAAAGAAACTGTTAACACA |
| | | AACACCTCTTTCTTAGCAAAACAGAAAGTGGGTATATATG |
| | | TGTGACAGACACAA |
| 18 | ACA48_st | TGTCCCTGACCTGGGTAGAGTGGCATCTGGTTGGTGATG |
| | | CCCATCTCATATCAGCCAGGGACAAAGCAACTCCTTGTT |
| | | CATCCCAGCTTGGCTTTTGATCCGTGCCCATGCCTGGTT |
| | | CATGCCTTGGACACATAG |
| 19 | ACA48_x_st | TGTCCCTGACCTGGGTAGAGTGGCATCTGGTTGGTGATG |
| | | CCCATCTCATATCAGCCAGGGACAAAGCAACTCCTTGTT |
| | | CATCCCAGCTTGGCTTTTGATCCGTGCCCATGCCTGGTT |
| | | CATGCCTTGGACACATAG |
| 20 | ACA51_x_st | GGCCTCCTGGTGCTTACCACAGGCTGTGTTCTTACACTG |
| | | ACTGTATAGAAAGAGGAGGTAGAGTAAACCTACCCCATA |
| | | TACACCTCAGCTCAGGCCCTGTGCCTGGTCTGTATTGTG |
| | | AATGGGGGAACATAG |
| 21 | ACA55_st | GAGCACCTGAATCTTTCCCATTCCTTGCTGCCTCGTGCC |
| | | GGTGTGGGGACAGATGGTGCTACAGAATGAGCAGAGGA |
| | | AATCCAGACAGGTTGTTTTCCATTTGTCTTGGGGCCTGTC |
| | | TCTACAGCTCTGCCACATTT |
| 22 | ACA57_st | TTGTCCTGGCCTATTTTTCTGCTCCCCTGTGCTCAGTTCT |
| | | AACAGGGTAGTCTGGCAGGACACACAGCAATTCCCTCTC |
| | | AGTTTAGGAGGGCCGTCCTAAGAATAGGGCTGGCTCTTA |
| | | AAGGCACGAGAGGACAATT |
| 23 | ACA61_st | ATCCTCCTGATCCCTTTCCCATCGGATCTGAACACTGGT |
| | | CTTGGTGGTCGTAAAAGGAGGAAAAGTAATAGTGAAGCT |
| | | GGCCTAAATGTTGTAATCTGGTATATGGCATGTGGGCTA |
| | | GTTTCAGACAGGT |
| 24 | ACA7_s_st | GACCTCCTGGGATCGCATCTGGAGAGTGCCTAGTATTCT |
| | | GCCAGCTTCGGAAAGGGAGGGAAAGCAAGCCTGGCAGA |
| | | GGCACCCATTCCATTCCCAGCTTGCTCCGTAGCTGGCGA |
| | | TTGGAAGACACTCTGCGACAGTG |
| 25 | ACA9_st | TAGCAAGCCTCCAGCGTGCTTGGGTCTGCGGTGACCCT |
| | | ATGCATTCCTTCAGTGCTTGCTAGAACAGTTTTGAAACGG |
| | | TTTGAGGCCTTGCCCTGCTCCATCCAGAGCAAGGTTATA |
| | | GAAATTTCAGACAATG |
| 26 | ENSG00000199282_st | TGGCAAGCCTCCAGCGTGCGTGGGTCTGTGGTGATGTG |
| | | TGCATTCCTACACTGCTTGCCAGAACAGTATTGAAATAGT |
| | | GGAGCGTTTGTCCTGCTCCATCCAGAGCAAGGTTATAGA |
| | | AATTTCAGACAATT |
| 27 | ENSG00000199411_s_st | TCTTAGTGACATAATTCTAATAGTTTGTTCCGACCTTCCA |
| | | CTGTGGACTCAATAGCAGGGAGATGAAGAGGACAGTGAT |
| | | TGCATGA |
| 28 | ENSG00000200879_st | TCACAATGATGAATGGTCCAAAACATTCGCGGTTTCCAC |
| | | CAGAATTCAAGGTGTTGGCAACTACCTTCCTTGGATGTCT |
| | | GAGTGA |
| 29 | ENSG00000200932_st | GCCGTGTGAGGCCACCTCTGCCATGCTGCAGGCTCCCC |
| | | AGCACGCAGTGGGGAACCCAACCTTGGAGAGCCGAGCC |
| | | GGG |
| 30 | ENSG00000201125_x_st | CTGGGGAATTCAAACTTGTGTTAAGAAAATGTGTCCCAGT |
| | | GTGCAATGGCTGCAAACAGCAGCTTCCTTGGTAGTGTAT |
| | | GCAGCCTGTTTGTTGTACGGGTTGCTCTAAAGGGCCTTG |
| | | GAGACAGTC |
| 31 | ENSG00000201299_x_st | ACTCACTGATGAGTAGCTTCTGACTTTCATTCTGAGTTTG |
| | | CTGAACCCAGATGCCATTCCTGGGAAGG |
| 32 | ENSG00000201859_x_st | CCTCATTGATTAGTAGCTTCTGACTTTTGTTCTGAGTTTG |
| | | CTGAAGCTAGATGCCATTCCAGATAAGA |
| 33 | ENSG00000202252_st | TCGCTGTGATGAGTGATTGTTAAACATTCGTAGTTTCCAC |
| | | CAAAAGCTTGGCTAATGATGGCAACACCTTCCTTGGATG |
| | | TCTGAGCGA |
| 34 | ENSG00000202498_x_st | AGATCATTGATGACTTCCATATATCCATTCCTTGGAAAGC |
| | | TGAACAACATGAGTGAAAACTCTACTGAAAAAAGAAAAGA |
| | | AATGGGAGGCCG |
| 35 | ENSG00000207002_st | TGCAGCTGTGTCAAATTTGGTGCCTGCCCTGCCTGTGTA |
| | | GGCACTGACCCAGAAGGCTGCCACAGAAACACTGACTC |
| | | ATGGGCCCTGTTCCTGTGTCTCGGGCTCAGGGATAAATT |
| | | TGGTTACAGATACCA |
| 36 | ENSG00000207002_x_st | TGCAGCTGTGTCAAATTTGGTGCCTGCCCTGCCTGTGTA |
| | | GGCACTGACCCAGAAGGCTGCCACAGAAACACTGACTC |
| | | ATGGGCCCTGTTCCTGTGTCTCGGGCTCAGGGATAAATT |
| | | TGGTTACAGATACCA |
| 37 | HBII-115_st | TGCCCAGTGATGACACCATCCTTGCTCCCCGTGCCCCCC |
| | | AGGGGCTATGGGCGACACCATGGCTGCCCCTGGGCTGG |
| | | GCCAGTGGGGCCAATGCCCAGGGGCTGAGGGCA |
| 38 | HBII-135_x_st | AAATGATGAAATCACCCAAAATAGCTGGAATTACCGGCA |
| | | GATTGTGTAGTGGTGAACCTATGGTTTTCTGAAG |
| 39 | HBII-180A_x_st | CCGGGGCCTCCATGATGTCCAGCACTGGGCTCCGACTG |
| | | CCACTGAGGACACGGTGCCCCCCGGGACCTTTGACACC |
| | | CGGGGGTCTGAGGGGCCCTGG |
| 40 | HBII-180C_x_st | CTGGGGCTCCCATGATGTCCAGCACTGGGCTCTGATCAC |
| | | CCCTGAGGACACAGTGCACCCCAGGACCTTTGACACCT |
| | | GGGGGTCTGAGGGGCCCCAG |
| 41 | HBII-202_st | CGCGTGATGACATTCTCCGGAATCGCTGTACGGCCTTGA |
| | | TGAAAGCACATTTGAACCCTTTTCCATCTGATT |
| 42 | HBII-239_st | TGTGTGTTGGAGGATGAAAGTACGGAGTGATCCATCGGC |
| | | TAAGTGTCTTGTCACAATGCTGACACTCAAACTGCTGACA |
| | | GCACACG |
| 43 | HBII-336_st | TGGCCAAGGATGAGAACTCTAATCTGATTTTATGTGCTTC |
| | | TGCTGTGATGGATTAAAGGATTTACCTGAGGCCA |
| 44 | HBII-429_st | GCTGTACATGATGACAACTGGCTCCCTCTACTGAACTGC |
| | | CATGAGGAAACTGCCATGTCACCCTTCTGATTACAGC |
| 45 | HBII-438A_s_st | GGATCGATGATGAGAATAATTGTCTGAGGATGCTGAGGG |
| | | ACTCATTCCAGATGTCAATCTGAGGTCC |
| 46 | HBII-55_st | TTGCAGTGATGACTTGCGAATCAAATCTGTCAATCCCCTG |
| | | AGTGCAATCACTGATGTCTCCATGTCTCTGAGCAA |
| 47 | HBII-85-11_st | TGGATCAATGATGACTTCCATACGTGGGTTCCTTGGAAA |
| | | GTTGAACAAAATGAGTGAAAACTTTATACTGTCATCCTCT |
| | | TCAAACTGAGGTCCA |
| 48 | HBII-85-26_st | TGGATCGATGATGACTATAAAAAAAATGGATCTCATCGGA |
| | | ATCTGAACAAAATGAGTGACCAAATCATTTCTGTGCCACT |
| | | TCTGTGAGCTGAGGTCCA |
| 49 | HBII-85-29_x_st | TGGATCGATGATGACTTAAAAAAATGGAAACCTTGGAAAT |
| | | CTGAACAAAATGAGTGACCAAGACACTTCTGTGAGCTGA |
| | | GGTCCA |
| 50 | HBII-85-2_x_st | TGGATCGATGATGAGTCCCCAAAAAAAACATTCCTTGGA |
| | | AAAGCTGAACAAAATGAGTGAAAACTCATACCGTCATTCT |
| | | CATCGGAACTGAGGTCCA |
| 51 | HBII-85-6_x_st | TGGATCGATGATGAGTCCTCCAAAAAAAACATTCCTTGGA |
| | | AAAGCTGAACAAAATGAGTGAAAACTCATACCGTCATTCT |
| | | CATCGGAACTGAGGTCCA |
| 52 | HBII-85-8_x_st | TGGATCGATGATGAGTCCTCCAAAAAAACATTCCTTGGAA |
| | | AAGCTGAACAAAATGAGTGAGAACTCATACCGTCGTTCT |
| | | CATCGGAACTGAGGTCCA |
| 53 | U104_st | GGCCTGCTGTGATGACATTCCAATTAAAGCACGTGTTAG |
| | | ACTGCTGACGCGGGTGATGCGAACTGGAGTCTGAGCCT |
| | | GCC |
| 54 | U13_st | GATCCTTTTGTAGTTCATGAGCGTGATGATTGGGTGTTCA |
| | | TACGCTTGTGTGAGATGTGCCACCCTTGAACCTTGTTAC |
| | | GACGTGGGCACATTACCCGTCTGACC |
| 55 | U17a_st | TCCAACGTGGATACACCCGGGAGGTCACTCTCCCCGGG |
| | | CTCTGTCCAAGTGGCGTAGGGGAGCATAGGGCTCTGCC |
| | | CCATGATGTACAAGTCCCTTTCCACAACGTTGGAAATAAA |
| | | GCTGGGCCTCGTGTCTGCGCCTGCATATTCCTACAGCTT |
| | | CCCAGAGTCCTGTCGACAATTACTGGGGAGACAAACCAT |
| | | GCAGGAAACAGCC |
| 56 | U17a_x_st | TCCAACGTGGATACACCCGGGAGGTCACTCTCCCCGGG |
| | | CTCTGTCCAAGTGGCGTAGGGGAGCATAGGGCTCTGCC |
| | | CCATGATGTACAAGTCCCTTTCCACAACGTTGGAAATAAA |
| | | GCTGGGCCTCGTGTCTGCGCCTGCATATTCCTACAGCTT |
| | | CCCAGAGTCCTGTCGACAATTACTGGGGAGACAAACCAT |
| | | GCAGGAAACAGCC |
| 57 | U17b_st | TCCAACGTGGATACCCTGGGAGGTCACTCTCCCCAGGCT |
| | | CTGTCCAAGTGGCATAGGGGAGCTTAGGGCTCTGCCCC |
| | | ATGATGTACAGTCCCTTTCCACAACGTTGAAGATGAAGCT |
| | | GGGCCTCGTGTCTGCGCCTGCATATTCCTACAGCTTCCC |
| | | AGAGTCCTGTGGACAATGACTGGGGAGACAAACCATGCA |
| | | GGAAACATAT |
| 58 | U17b_x_st | TCCAACGTGGATACCCTGGGAGGTCACTCTCCCCAGGCT |
| | | CTGTCCAAGTGGCATAGGGGAGCTTAGGGCTCTGCCCC |
| | | ATGATGTACAGTCCCTTTCCACAACGTTGAAGATGAAGCT |
| | | GGGCCTCGTGTCTGCGCCTGCATATTCCTACAGCTTCCC |
| | | AGAGTCCTGTGGACAATGACTGGGGAGACAAACCATGCA |
| | | GGAAACATAT |
| 59 | U22_st | TCCCAATGAAGAAACTTTCACATGTCTTACTCTCTGTCCT |
| | | AGTCCCAGAGCCTGTAAAGGTGAACCCACTGGGACTGG |
| | | CTGGGGGAGAAGAGGAAGATTTGTTCCAGAAGGAACTGT |
| | | CTGAGGGAT |
| 60 | U25_st | TTCCTATGATGAGGACCTTTTCACAGACCTGTACTGAGCT |
| | | CCGTGAGGATAAATAACTCTGAGGAGA |
| 61 | U26_st | CTACGGGGATGATTTTACGAACTGAACTCTCTCTTTCTGA |
| | | TGGATTAGTGGAGAAAACAGAAAATTCTGAGTAGC |
| 62 | U27_st | ACTCCATGATGAACACAAAATGACAAGCATATGGCTGAA |
| | | CTTTCAAGTGATGTCATCTTACTACTGAGAAGT |
| 63 | U28_st | GTCAGATGATTTGAATTGATAAGCTGATGTTCTGTGAGGT |
| | | ACAAAAGTTAATAGCATGTTAGAGTTCTGATGGCA |
| 64 | U29_st | TTTCTATGATGAATCAAACTAGCTCACTATGACCGACAGT |
| | | GAAAATACATGAACACCTGAGAAAC |
| 65 | U3-2_s_st | AAGACTATACTTTCAGGGATCATTTCTATAGTGTGTTACT |
| | | AGAGAAGTTTCTCTGAACGTGTAGAGCACCGAAAACCCC |
| | | GAGGAAGAGAGGTAGCGTTTTCTCCTGAGCGTGAAGCC |
| | | GGCTTTCTGGCGTTGCTTGGCTGCAACTGCCGTCAGCCA |
| | | TTGATGATCGTTCTTCTCTCCGTATTGGGGAGTGAGAGG |
| | | GAGAGAACGCGGTCTGAGTGGT |
| 66 | U30_st | GTTTGTGATGACTTACATGGAATCTCGTTCGGCTGATGA |
| | | CTTGCTGTTGAGACTCTGAAATCTGATTTTC |
| 67 | U31_x_st | CTCACCAGTGATGAGTTGAATACCGCCCCAGTCTGATCA |
| | | ATGTGTGACTGAAAGGTATTTTCTGAGCTGTG |
| 68 | U32A_x_st | GTCAGTGATGAGCAACATTCACCATCTTTCGTTTGAGTCT |
| | | CACGGCCATGAGATCAACCCCATGCACCGCTCTGAGA |
| 69 | U33_st | GGCCGGTGATGAGAACTTCTCCCACTCACATTCGAGTTT |
| | | CCCGACCATGAGATGACTCCACATGCACTACCATCTGAG |
| | | GCCAC |
| 70 | U34_st | CGTCCATGATGTTCCGCAACTACCTACATTGTTTGATCCT |
| | | CATGAAAGCAGCACTGGCTGAGACGC |
| 71 | U36A_st | TTGCAATGATGTGAATCTCTCACTGAATTCAACCTTGAAG |
| | | TGCGAATCCATGAGCTTTTTAACCCTGAGCAA |
| 72 | U38A_st | TTCTCGTGATGAAAACTCTGTCCAGTTCTGCTACTGAAGG |
| | | GAGAGAGATGAGAGCCTTTTAGGCTGAGGAA |
| 73 | U38B_st | TCTCAGTGATGAAAACTTTGTCCAGTTCTGCTACTGACAG |
| | | TAAGTGAAGATAAAGTGTGTCTGAGGAGA |
| 74 | U41_st | TGGGAAGTGATGACACCTGTGACTGTTGATGTGGAACTG |
| | | ATTTATCGCGTATTCGTACTGGCTGATCCTG |
| 75 | U43_x_st | CACAGATGATGAACTTATTGACGGGCGGACAGAAACTGT |
| | | GTGCTGATTGTCACGTTCTGATT |
| 76 | U48_st | AGTGATGATGACCCCAGGTAACTCTTGAGTGTGTCGCTG |
| | | ATGCCATCACCGCAGCGCTCTGACC |
| 77 | U49A_st | TGCTCTGATGAAATCACTAATAGGAAGTGCCGTCAGAAG |
| | | CGATAACTGACGAAGACTACTCCTGTCTGATT |
| 78 | U49A_x_st | TGCTCTGATGAAATCACTAATAGGAAGTGCCGTCAGAAG |
| | | CGATAACTGACGAAGACTACTCCTGTCTGATT |
| 79 | U49B_s_st | CTGATGATACTTGTAATAGGAAGTGCCGTCAGAAGCGAT |
| | | AACTGACGA |
| 80 | U49B_x_st | CTGATGATACTTGTAATAGGAAGTGCCGTCAGAAGCGAT |
| | | AACTGACGA |
| 81 | U51_st | GTTGCATGATGAATAAAATCAAATCACCATCTTTCGGCTG |
| | | AGTTCGTGATGGATTTGCTTTTTTCTGATT |
| 82 | U52_st | GGGAATGATGATTTCACAGACTAGAGTCTCCGATGCTGG |
| | | TCATGATGTCAAAACTAAGTTCTGA |
| 83 | U55_st | GTGTATGATGACAACTCGGTAATGCTGCATACTCCCGAG |
| | | TGCGCGGTGGGGAAGCCAACCTTGGAGAGCTGAGC |
| 84 | U55_x_st | GTGTATGATGACAACTCGGTAATGCTGCATACTCCCGAG |
| | | TGCGCGGTGGGGAAGCCAACCTTGGAGAGCTGAGC |
| 85 | U56_st | CCACAATGATGGCAATATTTTTCGTCAACAGCAGTTCACC |
| | | TAGTGAGTGTTGAGACTCTGGGTCTGAGTGA |
| 86 | U56_x_st | CCACAATGATGGCAATATTTTTCGTCAACAGCAGTTCACC |
| | | TAGTGAGTGTTGAGACTCTGGGTCTGAGTGA |
| 87 | U57_st | TGGAGGTGATGAACTGTCTGAGCCTGACCTTGTAGAATG |
| | | GAGGCAAAAAAACTGATTTAATGAGCCTGATCC |
| 88 | U59B_st | TATTCCTCACTGATGAGTACGTTCTGACTTTCGTTCTTCT |
| | | GAGTTTGCTGAAGCCAGATGCAATTTCTGAGAAGG |
| 89 | U64_st | ACTCTCTCGGCTCTGCATAGTTGCACTTGGCTTCACCCG |
| | | TGTGACTTTCGTAACGGGGAGAGAGAGAAAAGATCTCCT |
| | | CAGGACCTCGGATGGGCCTTACTGTGGCCTCTCTTTCCT |
| | | TGAGGGGTGCAACAGGC |
| 90 | U67_st | ATCCAAGGTGATTCCCTCTCCAAGGGGACATCAGTGCCT |
| | | CTCAGGAAAGTAGCAGCTTGGAATAGAATCTGGCATGCC |
| | | TAAGGCCTTTGGGGAACTGGGATGCTTATTTCCTCTGCC |
| | | TTCCTTGGCTGCCCACATGG |
| 91 | U67_x_st | ATCCAAGGTGATTCCCTCTCCAAGGGGACATCAGTGCCT |
| | | CTCAGGAAAGTAGCAGCTTGGAATAGAATCTGGCATGCC |
| | | TAAGGCCTTTGGGGAACTGGGATGCTTATTTCCTCTGCC |
| | | TTCCTTGGCTGCCCACATGG |
| 92 | U68_st | ATTGCACCTAAACCCAAGAATCACTGTTTCTTATAGCGGT |
| | | GGTTTAAACAGAGGTGCAAACAGCAAGCGGATCTTGTCG |
| | | CCTTTGGGGGGCTGTGGCCGTGCCCCTCAAAGTGAATTT |
| | | GGAGGTTCCACAACT |
| 93 | U68_x_st | ATTGCACCTAAACCCAAGAATCACTGTTTCTTATAGCGGT |
| | | GGTTTAAACAGAGGTGCAAACAGCAAGCGGATCTTGTCG |
| | | CCTTTGGGGGGCTGTGGCCGTGCCCCTCAAAGTGAATTT |
| | | GGAGGTTCCACAACT |
| 94 | U71a_st | CTCCTGCATCCGAAAGTGATCATAGGCTGCCTGTGCCTA |
| | | GGTCATTGATAGTGCAGGGAGAGGAACGCTGAAAGAGG |
| | | TTGTCCCCGTGTTTGGAGGGTCCACCCTATCCCATCCGA |
| | | ACCCTGAAGCTTTCACACAACT |
| 95 | U71b_x_st | TCCCTGTATTCGAAAGTGATCGTGGGCTGCCTTTGCCCT |
| | | GGTCATTGATAGTGCAGGGAGAGGAATCAATGAAAGCGC |
| | | TTCCCCGTGTTTGAAGGGTCCACTCCTATCCCTTCCAAA |
| | | CTCTGGAGCTTTCGTACATGC |
| 96 | U71d_st | CACCTGTATTCGAAAGTGATCGTGGGCTGCCTGTGCCCT |
| | | GGTCATTGATAGTGCAGGGAAAGAAATCGCGGAAAGTGC |
| | | TTCCCCGTGTTTGGAGGGTCCGCTCCTGTCCCTTTCAAA |
| | | CTCTGGAGCTTTCTCACACCT |
| 97 | U71d_x_st | CACCTGTATTCGAAAGTGATCGTGGGCTGCCTGTGCCCT |
| | | GGTCATTGATAGTGCAGGGAAAGAAATCGCGGAAAGTGC |
| | | TTCCCCGTGTTTGGAGGGTCCGCTCCTGTCCCTTTCAAA |
| | | CTCTGGAGCTTTCTCACACCT |
| 98 | U74_x_st | CTGCCTCTGATGAAGCCTGTGTTGGTAGGGACATCTGAG |
| | | AGTAATGATGAATGCCAACCGCTCTGATGGTGG |
| 99 | U75_st | AGCCTGTGATGCTTTAAGAGTAGTGGACAGAAGGGATTT |
| | | CTGAAATTCTATTCTGAGGCT |
| 100 | U78_s_st | GTGTAATGATGTTGATCAAATGTCTGACCTGAAATGAGCA |
| | | TGTAGACAAAGGTAACACTGAAGAA |
| 101 | U78_x_st | GTGTAATGATGTTGATCAAATGTCTGACCTGAAATGAGCA |
| | | TGTAGACAAAGGTAACACTGAAGAA |
| 102 | U83B_st | GCTGTTCAGTGATGAGGCCTGGAATGTGCGCTGGGCAC |
| | | AGCGCCCGAGACAGACTGCGGAACCGTTCCTTGTTGCC |
| | | TTCCTTCTGAGAACAGC |
| 103 | U83_st | GCCAAATGATGTTTATTTGAAACAGGAGCACCTCAGTGC |
| | | AAGGACGACTCTTATCTATCACCCATGACTGATGGCT |
| 104 | U89_st | CAGGCTGATGAGACTAAGGCGAATGCGACTCCGTGCTCT |
| | | CTGGCCCTTGGCTCCTTGTTGGGGGTGGGGACTACAGA |
| | | TGAGATCTGAAATCTTAGTGGTAGTACCTGAGCCATGAC |
| | | TCCCCACTGTAAGGCCAGATCAATAGCATTGGTGGCCTT |
| | | GCCTTCATTTCTGGTGCTGCCCCTAGTTCCTGGCAGCAG |
| | | CCTGCAGGGAGGCCCACAGGTGGGGTCCACGGTAGGG |
| | | CTGGGCACAAGCCACCTGAGCGCAACCTTGGATCTGAC |
| | | A |
| 105 | U8_x_st | ATCGTCAGGTGGGATAATCCTTACCTGTTCCTCCTCCGG |
| | | AGGGCAGATTAGAACATGATGATTGGAGATGCATGAAAC |
| | | GTGATTAACGTCTCTGCGTAATCAGGACTTGCAACACCC |
| | | TGATTGCTCCTGTCTGATT |
| 106 | U95_st | GCGGTGATGACCCCAACATGCCATCTGAGTGTCGGTGCT |
| | | GAAATCCAGAGGCTGTTTCTGAGC |
| 107 | hsa-miR-105_st | UCAAAUGCUCAGACUCCUGUGGU |
| 108 | hsa-miR-106a-star_st | CUGCAAUGUAAGCACUUCUUAC |
| 109 | hsa-miR-106a_st | AAAAGUGCUUACAGUGCAGGUAG |
| 110 | hsa-miR-106b-star_st | CCGCACUGUGGGUACUUGCUGC |
| 111 | hsa-miR-106b_st | UAAAGUGCUGACAGUGCAGAU |
| 112 | hsa-miR-1181_st | CCGUCGCCGCCACCCGAGCCG |
| 113 | hsa-miR-1183_st | CACUGUAGGUGAUGGUGAGAGUGGGCA |
| 114 | hsa-miR-1207-5p_st | UGGCAGGGAGGCUGGGAGGGG |
| 115 | hsa-miR-1228_st | UCACACCUGCCUCGCCCCCC |
| 116 | hsa-miR-1246_st | AAUGGAUUUUUGGAGCAGG |
| 117 | hsa-miR-124_st | UAAGGCACGCGGUGAAUGCC |
| 118 | hsa-miR-1254_st | AGCCUGGAAGCUGGAGCCUGCAGU |
| 119 | hsa-miR-125b-1-star_st | ACGGGUUAGGCUCUUGGGAGCU |
| 120 | hsa-miR-1268_st | CGGGCGUGGUGGUGGGGG |
| 121 | hsa-miR-126_st | UCGUACCGUGAGUAAUAAUGCG |
| 122 | hsa-miR-127-3p_st | UCGGAUCCGUCUGAGCUUGGCU |
| 123 | hsa-miR-127-5p_st | CUGAAGCUCAGAGGGCUCUGAU |
| 124 | hsa-miR-1271_st | CUUGGCACCUAGCAAGCACUCA |
| 125 | hsa-miR-1275_st | GUGGGGGAGAGGCUGUC |
| 126 | hsa-miR-1281_st | UCGCCUCCUCCUCUCCC |
| 127 | hsa-miR-128_st | UCACAGUGAACCGGUCUCUUU |
| 128 | hsa-miR-1299_st | UUCUGGAAUUCUGUGUGAGGGA |
| 129 | hsa-miR-1307_st | ACUCGGCGUGGCGUCGGUCGUG |
| 130 | hsa-miR-134_st | UGUGACUGGUUGACCAGAGGGG |
| 131 | hsa-miR-140-3p_st | UACCACAGGGUAGAACCACGG |
| 132 | hsa-miR-142-3p_st | UGUAGUGUUUCCUACUUUAUGGA |
| 133 | hsa-miR-142-5p_st | CAUAAAGUAGAAAGCACUACU |
| 134 | hsa-miR-143-star_st | GGUGCAGUGCUGCAUCUCUGGU |
| 135 | hsa-miR-143_st | UGAGAUGAAGCACUGUAGCUC |
| 136 | hsa-miR-145_st | GUCCAGUUUUCCCAGGAAUCCCU |
| 137 | hsa-miR-150_st | UCUCCCAACCCUUGUACCAGUG |
| 138 | hsa-miR-153_st | UUGCAUAGUCACAAAAGUGAUC |
| 139 | hsa-miR-154_st | UAGGUUAUCCGUGUUGCCUUCG |
| 140 | hsa-miR-155_st | UUAAUGCUAAUCGUGAUAGGGGU |
| 141 | hsa-miR-15a-star_st | CAGGCCAUAUUGUGCUGCCUCA |
| 142 | hsa-miR-15a_st | UAGCAGCACAUAAUGGUUUGUG |
| 143 | hsa-miR-17-star_st | ACUGCAGUGAAGGCACUUGUAG |
| 144 | hsa-miR-17_st | CAAAGUGCUUACAGUGCAGGUAG |
| 145 | hsa-miR-181a-2-star_st | ACCACUGACCGUUGACUGUACC |
| 146 | hsa-miR-181a-star_st | ACCAUCGACCGUUGAUUGUACC |
| 147 | hsa-miR-181a_st | AACAUUCAACGCUGUCGGUGAGU |
| 148 | hsa-miR-181b_st | AACAUUCAUUGCUGUCGGUGGGU |
| 149 | hsa-miR-181c-star_st | AACCAUCGACCGUUGAGUGGAC |
| 150 | hsa-miR-181c_st | AACAUUCAACCUGUCGGUGAGU |
| 151 | hsa-miR-181d_st | AACAUUCAUUGUUGUCGGUGGGU |
| 152 | hsa-miR-185_st | UGGAGAGAAAGGCAGUUCCUGA |
| 153 | hsa-miR-188-5p_st | CAUCCCUUGCAUGGUGGAGGG |
| 154 | hsa-miR-18a-star_st | ACUGCCCUAAGUGCUCCUUCUGG |
| 155 | hsa-miR-18a_st | UAAGGUGCAUCUAGUGCAGAUAG |
| 156 | hsa-miR-18b_st | UAAGGUGCAUCUAGUGCAGUUAG |
| 157 | hsa-miR-195-star_st | CCAAUAUUGGCUGUGCUGCUCC |
| 158 | hsa-miR-198_st | GGUCCAGAGGGGAGAUAGGUUC |
| 159 | hsa-miR-199a-5p_st | CCCAGUGUUCAGACUACCUGUUC |
| 160 | hsa-miR-199b-3p_st | ACAGUAGUCUGCACAUUGGUUA |
| 161 | hsa-miR-19a_st | UGUGCAAAUCUAUGCAAAACUGA |
| 162 | hsa-miR-19b_st | UGUGCAAAUCCAUGCAAAACUGA |
| 163 | hsa-miR-20a_st | UAAAGUGCUUAUAGUGCAGGUAG |
| 164 | hsa-miR-20b-star_st | ACUGUAGUAUGGGCACUUCCAG |
| 165 | hsa-miR-21-star_st | CAACACCAGUCGAUGGGCUGU |
| 166 | hsa-miR-214-star_st | UGCCUGUCUACACUUGCUGUGC |
| 167 | hsa-miR-223_st | UGUCAGUUUGUCAAAUACCCCA |
| 168 | hsa-miR-25-star_st | AGGCGGAGACUUGGGCAAUUG |
| 169 | hsa-miR-25_st | CAUUGCACUUGUCUCGGUCUGA |
| 170 | hsa-miR-27a_st | UUCACAGUGGCUAAGUUCCGC |
| 171 | hsa-miR-297_st | AUGUAUGUGUGCAUGUGCAUG |
| 172 | hsa-miR-299-3p_st | UAUGUGGGAUGGUAAACCGCUU |
| 173 | hsa-miR-29b-2-star_st | CUGGUUUCACAUGGUGGCUUAG |
| 174 | hsa-miR-29c-star_st | UGACCGAUUUCUCCUGGUGUUC |
| 175 | hsa-miR-29c_st | UAGCACCAUUUGAAAUCGGUUA |
| 176 | hsa-miR-320c_st | AAAAGCUGGGUUGAGAGGGU |
| 177 | hsa-miR-324-3p_st | ACUGCCCCAGGUGCUGCUGG |
| 178 | hsa-miR-331-5p_st | CUAGGUAUGGUCCCAGGGAUCC |
| 179 | hsa-miR-337-5p_st | GAACGGCUUCAUACAGGAGUU |
| 180 | hsa-miR-339-5p_st | UCCCUGUCCUCCAGGAGCUCACG |
| 181 | hsa-miR-33b-star_st | CAGUGCCUCGGCAGUGCAGCCC |
| 182 | hsa-miR-342-3p_st | UCUCACACAGAAAUCGCACCCGU |
| 183 | hsa-miR-342-5p_st | AGGGGUGCUAUCUGUGAUUGA |
| 184 | hsa-miR-346_st | UGUCUGCCCGCAUGCCUGCCUCU |
| 185 | hsa-miR-34b_st | CAAUCACUAACUCCACUGCCAU |
| 186 | hsa-miR-34c-3p_st | AAUCACUAACCACACGGCCAGG |
| 187 | hsa-miR-361-5p_st | UUAUCAGAAUCUCCAGGGGUAC |
| 188 | hsa-miR-362-5p_st | AAUCCUUGGAACCUAGGUGUGAGU |
| 189 | hsa-miR-363-star_st | CGGGUGGAUCACGAUGCAAUUU |
| 190 | hsa-miR-370_st | GCCUGCUGGGGUGGAACCUGGU |
| 191 | hsa-miR-376c_st | AACAUAGAGGAAAUUCCACGU |
| 192 | hsa-miR-377-star_st | AGAGGUUGCCCUUGGUGAAUUC |
| 193 | hsa-miR-379_st | UGGUAGACUAUGGAACGUAGG |
| 194 | hsa-miR-381_st | UAUACAAGGGCAAGCUCUCUGU |
| 195 | hsa-miR-382_st | GAAGUUGUUCGUGGUGGAUUCG |
| 196 | hsa-miR-409-3p_st | GAAUGUUGCUCGGUGAACCCCU |
| 197 | hsa-miR-409-5p_st | AGGUUACCCGAGCAACUUUGCAU |
| 198 | hsa-miR-410_st | AAUAUAACACAGAUGGCCUGU |
| 199 | hsa-miR-411_st | UAGUAGACCGUAUAGCGUACG |
| 200 | hsa-miR-423-3p_st | AGCUCGGUCUGAGGCCCCUCAGU |
| 201 | hsa-miR-423-5p_st | UGAGGGGCAGAGAGCGAGACUUU |
| 202 | hsa-miR-424-star_st | CAAAACGUGAGGCGCUGCUAU |
| 203 | hsa-miR-424_st | CAGCAGCAAUUCAUGUUUUGAA |
| 204 | hsa-miR-425-star_st | AUCGGGAAUGUCGUGUCCGCCC |
| 205 | hsa-miR-431_st | UGUCUUGCAGGCCGUCAUGCA |
| 206 | hsa-miR-432_st | UCUUGGAGUAGGUCAUUGGGUGG |
| 207 | hsa-miR-433_st | AUCAUGAUGGGCUCCUCGGUGU |
| 208 | hsa-miR-485-3p_st | GUCAUACACGGCUCUCCUCUCU |
| 209 | hsa-miR-485-5p_st | AGAGGCUGGCCGUGAUGAAUUC |
| 210 | hsa-miR-487a_st | AAUCAUACAGGGACAUCCAGUU |
| 211 | hsa-miR-487b_st | AAUCGUACAGGGUCAUCCACUU |
| 212 | hsa-miR-489_st | GUGACAUCACAUAUACGGCAGC |
| 213 | hsa-miR-491-3p_st | CUUAUGCAAGAUUCCCUUCUAC |
| 214 | hsa-miR-493_st | UGAAGGUCUACUGUGUGCCAGG |
| 215 | hsa-miR-494_st | UGAAACAUACACGGGAAACCUC |
| 216 | hsa-miR-495_st | AAACAAACAUGGUGCACUUCUU |
| 217 | hsa-miR-500-star_st | AUGCACCUGGGCAAGGAUUCUG |
| 218 | hsa-miR-500_st | UAAUCCUUGCUACCUGGGUGAGA |
| 219 | hsa-miR-501-3p_st | AAUGCACCCGGGCAAGGAUUCU |
| 220 | hsa-miR-501-5p_st | AAUCCUUUGUCCCUGGGUGAGA |
| 221 | hsa-miR-502-3p_st | AAUGCACCUGGGCAAGGAUUCA |
| 222 | hsa-miR-503_st | UAGCAGCGGGAACAGUUCUGCAG |
| 223 | hsa-miR-532-3p_st | CCUCCCACACCCAAGGCUUGCA |
| 224 | hsa-miR-532-5p_st | CAUGCCUUGAGUGUAGGACCGU |
| 225 | hsa-miR-543_st | AAACAUUCGCGGUGCACUUCUU |
| 226 | hsa-miR-551b-star_st | GAAAUCAAGCGUGGGUGAGACC |
| 227 | hsa-miR-574-3p_st | CACGCUCAUGCACACACCCACA |
| 228 | hsa-miR-574-5p_st | UGAGUGUGUGUGUGUGAGUGUGU |
| 229 | hsa-miR-584_st | UUAUGGUUUGCCUGGGACUGAG |
| 230 | hsa-miR-593-star_st | AGGCACCAGCCAGGCAUUGCUCAGC |
| 231 | hsa-miR-595_st | GAAGUGUGCCGUGGUGUGUCU |
| 232 | hsa-miR-611_st | GCGAGGACCCCUCGGGGUCUGAC |
| 233 | hsa-miR-613_st | AGGAAUGUUCCUUCUUUGCC |
| 234 | hsa-miR-615-3p_st | UCCGAGCCUGGGUCUCCCUCUU |
| 235 | hsa-miR-629-star_st | GUUCUCCCAACGUAAGCCCAGC |
| 236 | hsa-miR-629_st | UGGGUUUACGUUGGGAGAACU |
| 237 | hsa-miR-631_st | AGACCUGGCCCAGACCUCAGC |
| 238 | hsa-miR-647_st | GUGGCUGCACUCACUUCCUUC |
| 239 | hsa-miR-652_st | AAUGGCGCCACUAGGGUUGUG |
| 240 | hsa-miR-654-3p_st | UAUGUCUGCUGACCAUCACCUU |
| 241 | hsa-miR-660_st | UACCCAUUGCAUAUCGGAGUUG |
| 242 | hsa-miR-663b_st | GGUGGCCCGGCCGUGCCUGAGG |
| 243 | hsa-miR-665_st | ACCAGGAGGCUGAGGCCCCU |
| 244 | hsa-miR-671-5p_st | AGGAAGCCCUGGAGGGGCUGGAG |
| 245 | hsa-miR-758_st | UUUGUGACCUGGUCCACUAACC |
| 246 | hsa-miR-766_st | ACUCCAGCCCCACAGCCUCAGC |
| 247 | hsa-miR-767-5p_st | UGCACCAUGGUUGUCUGAGCAUG |
| 248 | hsa-miR-768-3p_st | UCACAAUGCUGACACUCAAACUGCUGAC |
| 249 | hsa-miR-768-5p_st | GUUGGAGGAUGAAAGUACGGAGUGAU |
| 250 | hsa-miR-769-3p_st | CUGGGAUCUCCGGGGUCUUGGUU |
| 251 | hsa-miR-769-5p_st | UGAGACCUCUGGGUUCUGAGCU |
| 252 | hsa-miR-874_st | CUGCCCUGGCCCGAGGGACCGA |
| 253 | hsa-miR-877-star_st | UCCUCUUCUCCCUCCUCCCAG |
| 254 | hsa-miR-877_st | GUAGAGGAGAUGGCGCAGGG |
| 255 | hsa-miR-923_st | GUCAGCGGAGGAAAAGAAACU |
| 256 | hsa-miR-92a-1-star_st | AGGUUGGGAUCGGUUGCAAUGCU |
| 257 | hsa-miR-92a-2-star_st | GGGUGGGGAUUUGUUGCAUUAC |
| 258 | hsa-miR-92a_st | UAUUGCACUUGUCCCGGCCUGU |
| 259 | hsa-miR-92b_st | UAUUGCACUCGUCCCGGCCUCC |
| 260 | hsa-miR-93-star_st | ACUGCUGAGCUAGCACUUCCCG |
| 261 | hsa-miR-936_st | ACAGUAGAGGGAGGAAUCGCAG |
| 262 | hsa-miR-938_st | UGCCCUUAAAGGUGAACCCAGU |
| 263 | hsa-miR-93_st | CAAAGUGCUGUUCGUGCAGGUAG |

### Biomarkers of Drug Responsiveness

Particular subsets of a patient population (e.g., a DLBCL patient population) may be responsive to a particular combination treatment. The invention provides sets of biomarkers (e.g., the biomarkers listed in Table 1), the expression levels of which (e.g., as detected in a biological sample obtained from a patient, such as a human subject in need of treatment) are indicative of responsiveness to the corresponding monotherapy and/or combination treatments (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP). The biomarkers were identified using methods similar to those previously described in, e.g., Chen et al. (Mol. Cancer Ther. 11:34-33, 2012), Wang et al. (J. Nat. Cancer Inst. 105: 1284-1291, 2013), and Knudsen et al. (PLoS One, 9: e87415, 2014), each of which are incorporated by reference herein in their entirety. In certain embodiments, an algorithm based on growth inhibition values (GI50) of a cell line (e.g., NCI60 cells) is subjected to treatment with a target drug and gene expression is determined (e.g., by microarray analysis, next generation sequencing, or NanoString). After normalization, genes with, e.g., a Pearson correlation greater than 0.25 or below −0.25 can be classified as biomarkers of sensitivity or resistance, respectively.

Biomarkers of responsiveness identified as described above can be used to predict responsiveness to drug combination treatment in subjects, e.g., patients suffering from or susceptible to a disease, disorder, or condition ((e.g., lymphoma (e.g., DLBCL)) treatable with the drug, by measuring their expression level in a biological sample obtained from the subject. For example, the expression level of one or more biomarkers, such as those shown in Table 1, can be measured in a biological sample obtained from the subject, and the resultant measurement can be used to assess the subject's responsiveness to a diversity of drug treatments and combination treatments (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP).

The expression level of a biomarker can be measured in a biological sample, e.g., using a device or method of the invention. For example, a microarray containing probes directed to one or more such biomarkers can be used to measure the mRNA expression level of the biomarkers. Each probe can have, e.g., at least 10, 15, 20, 25, 30 or more contiguous nucleic acid residues (e.g., at least 15) that are complementary to or identical to a nucleic acid sequence of a selected biomarker. The probe sequence can also be at least 85% (e.g., 90%, 95%, 99%, or 100%) identical to the sequence of the biomarker or its complement sequence. Preferably, the probe sequences are complementary to the nucleic acid sequence of the biomarker(s). In some embodiments, subjects showing elevated expression levels of a biomarker, e.g., relative to a control or the population median, are predicted to be more responsive to treatment with the drug combination, while subjects showing reduced expression of a biomarker, e.g., relative to a control or the population median, are predicted to be less responsive to treatment with the drug. In one embodiment, subjects with an elevated level of hsa-miR-766_st and/or hsa-miR-34b_st expression relative to the population median are predicted to be more responsive to COPE, while subjects with an expression level of hsa-miR-766_st and/or hsa-miR-34b_st below the population median are predicted to be less responsive to treatment with COPE. In other embodiments, subjects showing elevated expression levels of one or more of the biomarkers shown in Table 1 for the indicated therapy, e.g., relative to a control or the population median, are predicted to be less responsive to treatment with the indicated drug or drug combination, while subjects showing reduced expression levels of one or more of the biomarkers, e.g., relative to the population median, are predicted to be more responsive to treatment with the indicated drug or drug combination.

The expression level of multiple biomarkers (e.g., hsa-miR-766_st and hsa-miR-34b_st) in a biological sample (e.g., a biological sample obtained from the patient (e.g., a biopsy sample)) can be compared to: (i) each other, (ii) a predetermined cutoff level (for example, the expression level of a control, a median expression level observed in a population of controls, or the median expression level in the overall population), or (iii) an expression level in patients that are responsive to drug treatment or combination treatment (e.g., one or more of COPE, MBVP, MTX, DHAP, ICE, VIM, MIME, ESHAP, CBV, EPOCH, FCM, CVAD-A, CVAD-B, MINE, MOPP, CHOEP, BeICHOP, doxorubicin, bendamustine, ABVD, CHOP, prednisolone, and GemOx). Such comparisons can be used to determine a subject's responsiveness to one or more of the drug treatments shown in Table 1, which may be administered to a cancer patient, e.g., for treatment of lymphoma (e.g., DLBCL).

In some embodiments, a minimum difference in expression level of a biomarker (e.g., hsa-miR-766_st and/or hsa-miR-34b_st) between a patient and a control (e.g., a population median) can be sufficient to classify the subject as responsive to a drug combination or drug treatment. In certain embodiments, the minimum difference is a statistically significant difference (e.g., as determined according to statistical methods well known in the art, such as a t-test or a Wilcoxon rank test). In various embodiments, the minimum difference is at least about 1% to about 20% (e.g., 1, 2, 3, 4, 5, 6, 7, 8, 9, 10, 11, 12, 13, 14, 15, 16, 17, 18, 19, or 20%). In other embodiments, a maximum difference in expression level of a biomarker (e.g., hsa-miR-766_st and/or hsa-miR-34b_st) between a patient and a control (e.g., a population median) can be sufficient to classify the subject as responsive to the drug or drug combination treatment. In certain embodiments, the maximum difference is a statistically significant difference (e.g., as determined according to statistical methods well known in the art, such as a t-test or a Wilcoxon rank test). In various embodiments, the maximum difference is at least about 20% to about 100% (e.g., 20, 25, 30, 35, 40, 45, 50, 55, 60, 65, 70, 75, 80, 85, 90, or 100%). In specific embodiments, the survival of patients (e.g., lymphoma patients (e.g., patients with DLBCL)) for a certain amount of time after treatment may be calculated from lifetime data to estimate the survival function of patients (e.g., a Kaplan-Meier curve) after drug treatment or combination treatment (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP).

hsa-miR-766_st

The biomarker hsa-miR-766_st may be used to assess a cancer patient's (e.g, a lymphoma patient, such as a patient with DLBCL) responsiveness to a cancer treatment (e.g., treatment with, one or more of, e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, and prednisolone). The expression level of hsa-miR-766_st or changes in the nucleic acid sequence of hsa-miR-766_st may be assessed using nucleic acid amplification methods (e.g., PCR) using the paired forward and reverse primers of the invention (SEQ ID NO. 246, listed in Table 2). In the methods of the invention, the expression level of hsa-miR-766_st biomarker may increase or decrease in a biological sample from a cancer patient, (e.g, a lymphoma patient, such as a patient with DLBCL), relative to a control or a population median. Such changes in hsa-miR-766_st expression level can be used to determine the cancer patient's responsiveness to a diversity of drug treatments and combination treatments (e.g., treatment with, e.g., one or more of COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, and prednisolone). The methods of the invention may be used to determine such changes in the level of expression of hsa-miR-766_st, and the detection of such changes in expression level can be used to determine the responsiveness of the cancer patient to drug treatments or combination treatments of the invention (e.g., treatment with, e.g., one or more of COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, and prednisolone).

The biomarker hsa-miR-766_st may be used alone to predict cancer patient responsiveness (e.g, a lymphoma patient, such as a DLBCL patient) to treatment with COPE or in combination with one or more additional biomarkers (e.g., one, two, three, four, five, ten, or all of the biomarkers shown in Table 1 for COPE therapy), e.g., one or more of hsa-miR-34b_st, ACA48_x_st, ENSG00000202498_x_st, HBII-85-26_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with MBVP or in combination with one or more additional biomarkers (e.g., one, two, three, four, five, ten, twenty, or all of the biomarkers shown in Table 1 for MBVP therapy), e.g., one or more of hsa-miR-34b_st, ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with DHAP or in combination with one or more additional biomarkers (e.g., one, two, three, four, five, ten, twenty, or all of the biomarkers shown in Table 1 for DHAP therapy), e.g., one or more of hsa-miR-34b_st, ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2_s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with ICE or in combination with one or more additional biomarkers (e.g., one, two, three, four, five, ten, twenty, or all of the biomarkers shown in Table 1 for ICE therapy), e.g., one or more of hsa-miR-34b_st, 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with VIM or in combination with one or more additional biomarkers (e.g., one, two, three, four, five, ten, twenty, or all of the biomarkers shown in Table 1 for VIM therapy), e.g., one or more of hsa-miR-34b_st, ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with MIME or in combination with one or more additional biomarkers (e.g., one, two, three, four, five, ten, twenty, or all of the biomarkers shown in Table 1 for MIME therapy), e.g., one or more of hsa-miR-34b_st, ACA10_s_st, ACA11_st, ACA13_st, ACA18_x_st, ACA21_st, ACA40_x_st, ACA48_x_st, ACA51_x_st, ACA55_st, ACA61_st, ACA7_s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with ESHAP or in combination with one or more additional biomarkers (e.g., one, two, three, four, five, ten, twenty, or all of the biomarkers shown in Table 1 for ESHAP therapy), e.g., one or more of hsa-miR-34b_st, ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and/or hsa-miR-768-5p_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with CBV or in combination with one or more additional biomarkers (e.g., one, two, three, four, five, ten, twenty, or all of the biomarkers shown in Table 1 for CBV therapy), e.g., one or more of hsa-miR-34b_st, ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with EPOCH or in combination with one or more additional biomarkers (e.g., one, two, three, four, five, ten, twenty, or all of the biomarkers shown in Table 1 for EPOCH therapy), e.g., one or more of hsa-miR-34b_st, ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with FCM or in combination with one or more additional biomarkers (e.g., one, two, three, four, five, ten, twenty, or all of the biomarkers shown in Table 1 for FCM therapy), e.g., one or more of hsa-miR-34b_st, ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with CVAD-A or in combination with one or more additional biomarkers (e.g., one, two, three, four, five, ten, twenty, or all of the biomarkers shown in Table 1 for CVAD-A therapy), e.g., one or more of hsa-miR-34b_st, ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and/or hsa-miR-92a-2-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with CVAD-B or in combination with one or more additional biomarkers (e.g., one, two, three, four, five, ten, twenty, or all of the biomarkers shown in Table 1 for CVAD-B therapy), e.g., one or more of hsa-miR-34b_st, ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_st, hsa-miR-663b_st, hsa-miR-768-5p_st, and/or hsa-miR-92a_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with MINE or in combination with one or more additional biomarkers (e.g., one, two, three, four, five, ten, twenty, or all of the biomarkers shown in Table 1 for MINE therapy), e.g., one or more of hsa-miR-34b_st, 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with MOPP or in combination with one or more additional biomarkers (e.g., one, two, three, four, five, ten, twenty, or all of the biomarkers shown in Table 1 for MOPP therapy), e.g., one or more of hsa-miR-34b_st, ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with CHOEP or in combination with one or more additional biomarkers (e.g., one, two, three, four, five, ten, twenty, or all of the biomarkers shown in Table 1 for CHOEP therapy), e.g., one or more of hsa-miR-34b_st, ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with BeICHOP or in combination with one or more additional biomarkers (e.g., one, two, three, four, five, ten, twenty, or all of the biomarkers shown in Table 1 for BeICHOP therapy), e.g., one or more of hsa-miR-34b_st, ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with MTX or in combination with one or more additional biomarkers (e.g., one, two, three, four, five, ten, twenty, or all of the biomarkers shown in Table 1 for doxorubicin therapy), e.g., one or more of, e.g., hsa-miR-34b_st, ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and/or hsa-miR-936_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with doxorubicin or in combination with one or more additional biomarkers (e.g., one, two, three, four, five, ten, twenty, or all of the biomarkers shown in Table 1 for doxorubicin therapy), e.g., one or more of, e.g., hsa-miR-34b_st, ACA13_st, ACA48_x_st, U104_st, U55_st, U55_x_st, U74_x_st, hsa-miR-106a-star_st, hsa-miR-106b-star_st, hsa-miR-106b_st, hsa-miR-124_st, hsa-miR-1281_st, hsa-miR-1299_st, hsa-miR-140-3p_st, hsa-miR-195-star_st, hsa-miR-297_st, hsa-miR-29b-2-star_st, hsa-miR-33b-star_st, hsa-miR-342-3p_st, hsa-miR-342-5p_st, hsa-miR-432_st, hsa-miR-550-star_st, hsa-miR-629-star_st, hsa-miR-629_st, hsa-miR-652_st, hsa-miR-654-3p_st, hsa-miR-671-5p_st, hsa-miR-768-3p_st, hsa-miR-877-star_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with bendamustine or in combination with one or more additional biomarkers (e.g., one, two, three, four, five, ten, or all of the biomarkers shown in Table 1 for bendamustine therapy), e.g., one or more of hsa-miR-34b_st, ACA11_st, ACA24_x_st, ACA7_s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with ABVD or in combination with one or more additional biomarkers (e.g., one, two, three, four, five, ten, twenty, or all of the biomarkers shown in Table 1 for ABVD therapy), e.g., one or more of e.g., 14qII-14_st, 14qII-14_x_st, 14qII-1_st, 14qII-1_x_st, 14qII-22_x_st, 14qII-26_st, 14qII-26_x_st, HBII-85-26_st, HBII-85-6_x_st, U22_st, U48_st, U55_st, U55_x_st, hsa-miR-124_st, hsa-miR-125b-1-star_st, hsa-miR-127-3p_st, hsa-miR-1271_st, hsa-miR-1281_st, hsa-miR-1299_st, hsa-miR-134_st, hsa-miR-140-3p_st, hsa-miR-143_st, hsa-miR-145_st, hsa-miR-154_st, hsa-miR-195-star_st, hsa-miR-199a-5p_st, hsa-miR-199b-3p_st, hsa-miR-299-3p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-495_st, hsa-miR-629-star_st, hsa-miR-654-3p_st, hsa-miR-665_st, hsa-miR-671-5p_st, hsa-miR-758_st, and/or hsa-miR-923_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with CHOP or in combination with one or more additional biomarkers (e.g., one, two, three, four, five, ten, twenty, or all of the biomarkers shown in Table 1 for CHOP therapy), e.g., one or more of ACA48_x_st, U55_x_st, hsa-miR-106b-star_st, hsa-miR-106b_st, hsa-miR-1181_st, hsa-miR-124_st, hsa-miR-1299_st, hsa-miR-25-star_st, hsa-miR-33b-star_st, hsa-miR-432_st, hsa-miR-551b-star_st, hsa-miR-629-star_st, hsa-miR-629_st, hsa-miR-652_st, hsa-miR-654-3p_st, hsa-miR-671-5p_st, hsa-miR-877-star_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-766_st may be used alone to predict cancer patient responsiveness (e.g, a lymphoma patient, such as a DLBCL patient) to treatment with prednisolone or in combination with one or more additional biomarkers (e.g., one, two, three, four, five, ten, twenty, or all of the biomarkers shown in Table 1 for prednisolone therapy), e.g., one or more of ACA18_x_st, ACA24_s_st, ACA24_x_st, ACA25_x_st, ACA3-2_st, ACA3_st, ACA40_x_st, ACA47_st, ACA48_st, ACA52_st, ACA54_st, ACA62_st, ACA7_s_st, ENSG00000199411_s_st, ENSG00000200394_st, ENSG00000200879_st, ENSG00000207002_st, HBII-180A_x_st, HBII-202_st, HBII-239_st, HBII-382_s_st, HBII-419_st, HBII-429_st, HBII-52-32_x_st, HBII-55_st, U104_st, U17a_st, U17b_st, U17b_x_st, U25_st, U26_st, U27_st, U28_st, U29_st, U3-2_s_st, U30_st, U31_st, U31_x_st, U33_st, U35A_st, U36A_x_st, U38A_st, U38B_st, U41_st, U46_st, U46_x_st, U49A_st, U50B_st, U50B_x_st, U50_st, U55_st, U56_st, U56_x_st, U57_st, U67_st, U67_x_st, U68_st, U68_x_st, U70_x_st, U73a_st, U78_s_st, U78_x_st, U83_st, U93_st, U95_st, hsa-miR-1181_st, hsa-miR-1207-5p_st, hsa-miR-1246_st, hsa-miR-1268_st, hsa-miR-1275_st, hsa-miR-17-star_st, hsa-miR-17_st, hsa-miR-195-star_st, hsa-miR-19b_st, hsa-miR-297_st, hsa-miR-571_st, hsa-miR-768-3p_st, hsa-miR-768-5p_st, hsa-miR-877-star_st, hsa-miR-92a-1-star_st, hsa-miR-92a-2-star_st, hsa-miR-92a_st, hsa-miR-938_st, and/or hsa-miR-939_st. The expression level of the biomarker(s) may be determined using, e.g., a microarray, PCR, or other techniques described herein, for example, using a nucleic acid probe sequence based on the target sequences shown in Table 2.

hsa-miR-34b_st

The biomarker hsa-miR-34b_st may be used to assess a cancer patient's (e.g, a lymphoma patient, such as a patient with DLBCL) responsiveness to a cancer treatment (e.g., treatment with, one or more of e.g., COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, and GemOx). The expression level of hsa-miR-34b_st or changes in the nucleic acid sequence of hsa-miR-34b_st may be assessed using nucleic acid amplification methods (e.g., PCR) using the paired forward and reverse primers of the invention (SEQ ID NO. 185, listed in Table 2). In the methods of the invention, the expression level of hsa-miR-34b_st biomarker may increase or decrease in a biological sample from a cancer patient, (e.g, a lymphoma patient, such as a patient with DLBCL), relative to a control or a population median. Such changes in hsa-miR-34b_st expression level can be used to determine the cancer patient's responsiveness to a diversity of drug treatments and combination treatments (e.g., treatment with, e.g., one or more of COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, ABVD, CHOP, and prednisolone). The methods of the invention may be used to determine such changes in the level of expression of hsa-miR-34b_st, and the detection of such changes in expression level can be used to determine the responsiveness of the cancer patient to drug treatments or combination treatments of the invention (e.g., treatment with, e.g., one or more of COPE, MBVP, DHAP, ICE, VIM, MIME, ESHAP, CBV, EPOCH, FCM, CVAD-A, CVAD-B, MINE, MOPP, CHOEP, BeICHOP, MTX, doxorubicin, bendamustine, and GemOx).

The biomarker hsa-miR-34b_st may be used alone to predict cancer patient responsiveness (e.g, a lymphoma patient, such as a DLBCL patient) to treatment with COPE or in combination with one or more additional biomarkers (e.g., one, two, three, four, five, ten, or all of the biomarkers shown in Table 1 for COPE therapy), e.g., one or more of, e.g., hsa-miR-766_st, ACA48_x_st, ENSG00000202498_x_st, HBII-85-26_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with MBVP or in combination with one or more additional biomarkers (e.g., one, two, three, four, five, ten, twenty, or all of the biomarkers shown in Table 1 for MBVP therapy), e.g., one or more of hsa-miR-766_st, ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with DHAP or in combination with one or more additional biomarkers (e.g., one, two, three, four, five, ten, twenty, or all of the biomarkers shown in Table 1 for DHAP therapy), e.g., one or more of hsa-miR-766_st, ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2_s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with ICE or in combination with one or more additional biomarkers (e.g., one, two, three, four, five, ten, twenty, or all of the biomarkers shown in Table 1 for ICE therapy), e.g., one or more of hsa-miR-766_st, 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with VIM or in combination with one or more additional biomarkers (e.g., one, two, three, four, five, ten, twenty, or all of the biomarkers shown in Table 1 for VIM therapy), e.g., one or more of hsa-miR-766_st, ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with MIME or in combination with one or more additional biomarkers (e.g., one, two, three, four, five, ten, twenty, or all of the biomarkers shown in Table 1 for MIME therapy), e.g., one or more of hsa-miR-766_st, ACA10_s_st, ACA11_st, ACA13_st, ACA18_x_st, ACA21_st, ACA40_x_st, ACA48_x_st, ACA51_x_st, ACA55_st, ACA61_st, ACA7s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_{—}st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with ESHAP or in combination with one or more additional biomarkers (e.g., one, two, three, four, five, ten, twenty, or all of the biomarkers shown in Table 1 for ESHAP therapy), e.g., one or more of hsa-miR-766_st, ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and/or hsa-miR-768-5p_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with CBV or in combination with one or more additional biomarkers (e.g., one, two, three, four, five, ten, twenty, or all of the biomarkers shown in Table 1 for CBV therapy), e.g., one or more of hsa-miR-766_st, ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with EPOCH or in combination with one or more additional biomarkers (e.g., one, two, three, four, five, ten, twenty, or all of the biomarkers shown in Table 1 for EPOCH therapy), e.g., one or more of hsa-miR-766_st, ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with FCM or in combination with one or more additional biomarkers (e.g., one, two, three, four, five, ten, twenty, or all of the biomarkers shown in Table 1 for FCM therapy), e.g., one or more of hsa-miR-766, ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with CVAD-A or in combination with one or more additional biomarkers (e.g., one, two, three, four, five, ten, twenty, or all of the biomarkers shown in Table 1 for CVAD-A therapy), e.g., one or more of hsa-miR-766, ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and/or hsa-miR-92a-2-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with CVAD-B or in combination with one or more additional biomarkers (e.g., one, two, three, four, five, ten, twenty, or all of the biomarkers shown in Table 1 for CVAD-B therapy), e.g., one or more of hsa-miR-766_st, ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_{—}st, hsa-miR-663b_st, hsa-miR-768-5p_st, and/or hsa-miR-92a_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with MINE or in combination with one or more additional biomarkers (e.g., one, two, three, four, five, ten, twenty, or all of the biomarkers shown in Table 1 for MINE therapy), e.g., one or more of hsa-miR-766_st, 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and/or hsa-miR-938_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with MOPP or in combination with one or more additional biomarkers (e.g., one, two, three, four, five, ten, twenty, or all of the biomarkers shown in Table 1 for MOPP therapy), e.g., one or more of hsa-miR-766_st, ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with CHOEP or in combination with one or more additional biomarkers (e.g., one, two, three, four, five, ten, twenty, or all of the biomarkers shown in Table 1 for CHOEP therapy), e.g., one or more of hsa-miR-766_st, ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with BeICHOP or in combination with one or more additional biomarkers (e.g., one, two, three, four, five, ten, twenty, or all of the biomarkers shown in Table 1 for BeICHOP therapy), e.g., one or more of hsa-miR-766_st, ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with MTX or in combination with one or more additional biomarkers (e.g., one, two, three, four, five, ten, twenty, or all of the biomarkers shown in Table 1 for doxorubicin therapy), e.g., one or more of, e.g., hsa-miR-766_st, ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and/or hsa-miR-936_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with bendamustine or in combination with one or more additional biomarkers (e.g., one, two, three, four, five, ten, or all of the biomarkers shown in Table 1 for bendamustine therapy), e.g., one or more of hsa-miR-766_st, ACA11_st, ACA24_x_st, ACA7_s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_{—}st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

The biomarker hsa-miR-34b_st may be used alone to predict cancer patient responsiveness (e.g, a lymphoma patient, such as a DLBCL patient) to treatment with GemOx or in combination with one or more additional biomarkers (e.g., one, two, three, four, five, ten, twenty, or all of the biomarkers shown in Table 1 for GemOx therapy), e.g., one or more of hsa-miR-126_st, hsa-miR-153_st, hsa-miR-19b_st, hsa-miR-424-star_st, hsa-miR-491-3p_st, and/or hsa-miR-503_st. The expression level of the biomarker(s) may be determined using, e.g., a microarray, PCR, or other techniques described herein, for example, using a nucleic acid probe sequence based on the target sequences shown in Table 2.

### Additional Biomarkers

Additional biomarkers that may be used in the methods of the invention to assess the responsiveness of a cancer patient (e.g., a lymphoma patient, such as a DLBCL patient) to treatment for cancer may include, but are not limited to the biomarkers listed in Table 1. The expression level of these biomarkers or changes in the nucleic acid sequence of these biomarkers may be assessed using nucleic acid amplification methods (e.g., PCR) using the paired forward and reverse primers of the invention (SEQ ID NOs. 1-263, listed in Table 2). In the methods of the invention, the expression level of these biomarkers may increase or decrease in a biological sample from a cancer patient, (e.g, a lymphoma patient, such as a patient with DLBCL), relative to a control or a population median. Such changes in biomarker (e.g., the biomarkers listed in Table 1) expression level can be used to determine the cancer patient's responsiveness to a diversity of drug treatments and combination treatments (e.g., treatment with e.g., one or more of CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP). The methods of the invention may be used to determine such changes in the level of expression of one or more biomarkers (e.g., one, two, three, four, five, ten, or all of the biomarkers shown in Table 1), and the detection of such changes in expression level can be used to determine the responsiveness of the cancer patient to drug treatments or combination treatments of the invention (e.g., treatment with, e.g., one or more of CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP).

Each of the biomarkers shown in Table 1 for CVP therapy (e.g., hsa-miR-106b-star_st, hsa-miR-1181_st, hsa-miR-25-star_st, hsa-miR-432_st, hsa-miR-551b-star_st, hsa-miR-652_st, hsa-miR-671-5p_st, and/or hsa-miR-93-star_st) may be used alone to predict cancer patient responsiveness (e.g, a lymphoma patient, such as a DLBCL patient) to treatment with CVP or in combination with one or more additional biomarkers (e.g., one, two, three, four, five, or all of the biomarkers shown in Table 1 for CVP therapy), e.g., one or more of hsa-miR-106b-star_st, hsa-miR-1181_st, hsa-miR-25-star_st, hsa-miR-432_st, hsa-miR-551b-star_st, hsa-miR-652_st, hsa-miR-671-5p_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for cyclophosphamide therapy (e.g., ENSG00000200879_st, ENSG00000202252_st, HBII-142_st, HBII-142_x_st, HBII-202_st, U42A_st, U59B_st, hsa-miR-1202_st, hsa-miR-184_st, hsa-miR-187_st, hsa-miR-191_st, hsa-miR-196a_st, hsa-miR-203_st, hsa-miR-29b-2-star_st, hsa-miR-29c-star_st, hsa-miR-328_st, hsa-miR-375_st, hsa-miR-423-3p_st, hsa-miR-423-5p_st, hsa-miR-449a_st, hsa-miR-449b_st, hsa-miR-489_st, and/or hsa-miR-768-3p_st) may be used alone to predict cancer patient responsiveness (e.g, a lymphoma patient, such as a DLBCL patient) to treatment with cyclophosphamide or in combination with one or more additional biomarkers (e.g., one, two, three, four, five, ten, twenty, or all of the biomarkers shown in Table 1 for cyclophosphamide therapy), e.g., one or more of ENSG00000200879_st, ENSG00000202252_st, HBII-142_st, HBII-142_x_st, HBII-202_st, U42A_st, U59B_st, hsa-miR-1202_st, hsa-miR-184_st, hsa-miR-187_st, hsa-miR-191_st, hsa-miR-196a_st, hsa-miR-203_st, hsa-miR-29b-2-star_st, hsa-miR-29c-star_st, hsa-miR-328_st, hsa-miR-375_st, hsa-miR-423-3p_st, hsa-miR-423-5p_st, hsa-miR-449a_st, hsa-miR-449b_st, hsa-miR-489_st, and/or hsa-miR-768-3p_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for vincristine therapy (e.g., hsa-miR-106b-star_st, hsa-miR-25-star_st, hsa-miR-432_st, hsa-miR-551b-star_st, hsa-miR-671-5p_st, and/or hsa-miR-93-star_st) may be used alone to predict cancer patient responsiveness (e.g, a lymphoma patient, such as a DLBCL patient) to treatment with vincristine or in combination with one or more additional biomarkers (e.g., one, two, three, four, five, or all of the biomarkers shown in Table 1 for vincristine therapy), e.g., one or more of hsa-miR-106b-star_st, hsa-miR-25-star_st, hsa-miR-432_st, hsa-miR-551b-star_st, hsa-miR-671-5p_st, and/or hsa-miR-93-star_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for BEACOPP therapy (e.g., 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-85-26_st, HBII-85-6_x_st, hsa-miR-106b-star_st, hsa-miR-124_st, hsa-miR-127-3p_st, hsa-miR-127-5p_st, hsa-miR-1281_st, hsa-miR-1299_st, hsa-miR-134_st, hsa-miR-140-3p_st, hsa-miR-154_st, hsa-miR-181a-star_st, hsa-miR-195-star_st, hsa-miR-199a-5p_st, hsa-miR-214-star_st, hsa-miR-299-3p_st, hsa-miR-337-5p_st, hsa-miR-339-5p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-410_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-615-3p_st, hsa-miR-654-3p_st, hsa-miR-665_st, hsa-miR-671-5p_st, hsa-miR-768-3p_st, and/or hsa-miR-92b_st) may be used alone to predict cancer patient responsiveness (e.g, a lymphoma patient, such as a DLBCL patient) to treatment with BEACOPP or in combination with one or more additional biomarkers (e.g., one, two, three, four, five, ten, twenty, or all of the biomarkers shown in Table 1 for BEACOPP therapy), e.g., one or more of 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-85-26_st, HBII-85-6_x_st, hsa-miR-106b-star_st, hsa-miR-124_st, hsa-miR-127-3p_st, hsa-miR-127-5p_st, hsa-miR-1281_st, hsa-miR-1299_st, hsa-miR-134_st, hsa-miR-140-3p_st, hsa-miR-154_st, hsa-miR-181a-star_st, hsa-miR-195-star_st, hsa-miR-199a-5p_st, hsa-miR-214-star_st, hsa-miR-299-3p_st, hsa-miR-337-5p_st, hsa-miR-339-5p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-410_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-615-3p_st, hsa-miR-654-3p_st, hsa-miR-665_st, hsa-miR-671-5p_st, hsa-miR-768-3p_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for CEPP therapy (e.g., 14qII-12_st, 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-336_st, HBII-85-26_st, HBII-85-6_x_st, hsa-miR-125b-1-star_st, hsa-miR-127-3p_st, hsa-miR-127-5p_st, hsa-miR-1271_st, hsa-miR-1281_st, hsa-miR-134_st, hsa-miR-140-3p_st, hsa-miR-143-star_st, hsa-miR-143_st, hsa-miR-145_st, hsa-miR-154_st, hsa-miR-155_st, hsa-miR-181a-star_st, hsa-miR-181a_st, hsa-miR-181b_st, hsa-miR-181c_st, hsa-miR-199a-5p_st, hsa-miR-199b-3p_st, hsa-miR-21-star_st, hsa-miR-214-star_st, hsa-miR-27a_st, hsa-miR-299-3p_st, hsa-miR-337-5p_st, hsa-miR-339-5p_st, hsa-miR-342-3p_st, hsa-miR-34c-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-494_st, hsa-miR-543_st, hsa-miR-574-3p_st, hsa-miR-615-3p_st, hsa-miR-654-3p_st, hsa-miR-665_st, hsa-miR-671-5p_st, hsa-miR-758_st, and/or hsa-miR-92b_st) may be used alone to predict cancer patient responsiveness (e.g, a lymphoma patient, such as a DLBCL patient) to treatment with CEPP or in combination with one or more additional biomarkers (e.g., one, two, three, four, five, ten, twenty or all of the biomarkers shown in Table 1 for CEPP therapy), e.g., one or more of 14qII-12_st, 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-336_st, HBII-85-26_st, HBII-85-6_x_st, hsa-miR-125b-1-star_st, hsa-miR-127-3p_st, hsa-miR-127-5p_st, hsa-miR-1271_st, hsa-miR-1281_st, hsa-miR-134_st, hsa-miR-140-3p_st, hsa-miR-143-star_st, hsa-miR-143_st, hsa-miR-145_st, hsa-miR-154_st, hsa-miR-155_st, hsa-miR-181a-star_st, hsa-miR-181a_st, hsa-miR-181b_st, hsa-miR-181c_st, hsa-miR-199a-5p_st, hsa-miR-199b-3p_st, hsa-miR-21-star_st, hsa-miR-214-star_st, hsa-miR-27a_st, hsa-miR-299-3p_st, hsa-miR-337-5p_st, hsa-miR-339-5p_st, hsa-miR-342-3p_st, hsa-miR-34c-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-494_st, hsa-miR-543_st, hsa-miR-574-3p_st, hsa-miR-615-3p_st, hsa-miR-654-3p_st, hsa-miR-665_st, hsa-miR-671-5p_st, hsa-miR-758_st, and/or hsa-miR-92b_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for COPP therapy (e.g., hsa-miR-106b-star_st, hsa-miR-1181_st, hsa-miR-432_st, hsa-miR-551b-star_st, hsa-miR-652_st, hsa-miR-671-5p_st, hsa-miR-93-star_st, and/or hsa-miR-93_st) may be used alone to predict cancer patient responsiveness (e.g, a lymphoma patient, such as a DLBCL patient) to treatment with COPP or in combination with one or more additional biomarkers (e.g., one, two, three, four, five, or all of the biomarkers shown in Table 1 for COPP therapy), e.g., one or more of hsa-miR-106b-star_st, hsa-miR-1181_st, hsa-miR-432_st, hsa-miR-551b-star_st, hsa-miR-652_st, hsa-miR-671-5p_st, hsa-miR-93-star_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

Each of the biomarkers shown in Table 1 for romidepsin therapy (e.g., hsa-miR-105_st, hsa-miR-106b_st, hsa-miR-185_st, hsa-miR-188-5p_st, hsa-miR-25_st, hsa-miR-320c_st, hsa-miR-362-5p_st, hsa-miR-500-star_st, hsa-miR-500_st, hsa-miR-501-3p_st, hsa-miR-501-5p_st, hsa-miR-502-3p_st, hsa-miR-532-3p_st, hsa-miR-532-5p_st, hsa-miR-584_st, hsa-miR-611_st, hsa-miR-652_st, hsa-miR-660_st, hsa-miR-767-5p_st, hsa-miR-769-3p_st, hsa-miR-877_st, and/or hsa-miR-93_st) may be used alone to predict cancer patient responsiveness (e.g, a lymphoma patient, such as a DLBCL patient) to treatment with romidepsin or in combination with one or more additional biomarkers (e.g., one, two, three, four, five, ten, twenty, or all of the biomarkers shown in Table 1 for romidepsin therapy), e.g., one or more of hsa-miR-105_st, hsa-miR-106b_st, hsa-miR-185_st, hsa-miR-188-5p_st, hsa-miR-25_st, hsa-miR-320c_st, hsa-miR-362-5p_st, hsa-miR-500-star_st, hsa-miR-500_st, hsa-miR-501-3p_st, hsa-miR-501-5p_st, hsa-miR-502-3p_st, hsa-miR-532-3p_st, hsa-miR-532-5p_st, hsa-miR-584_st, hsa-miR-611_st, hsa-miR-652_st, hsa-miR-660_st, hsa-miR-767-5p_st, hsa-miR-769-3p_st, hsa-miR-877_st, and/or hsa-miR-93_st. The expression level of the biomarker(s) may be determined using, e.g., a microarray, PCR, or other techniques described herein, for example, using a nucleic acid probe sequence based on the target sequences shown in Table 2.

### Methods for Identifying Biomarkers of Drug Responsiveness

The invention features methods for identifying biomarkers for predicting the responsiveness of a lymphoma patient (e.g., a patient with DLBCL) to a drug treatment or combination treatment of interest (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP). Such methods can involve, for example, an algorithm based on growth inhibition values (GI50) of cell lines (e.g., NCI60 cell lines) subjected to treatment with a drug or drug combination (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, or BeICHOP, or a metabolite thereof), followed by measurement of gene expression (e.g., using a microarray (e.g., an Affymetrix HG-U133A Genechip array)).

In certain embodiments, the gene expression measurements of NCI60 cancer cell lines are obtained from a publically available database (e.g., the National Cancer Institute and the Massachusetts Institute of Technology). Each dataset can be normalized so that sample expression measured by different chips can be compared. The preferred method of normalization is the log it transformation, which may be performed for each gene y on each chip, as follows:

log it(y)=log [(y−background)/(saturation−y)],

where background is calculated as the minimum intensity measured on the chip minus 0.1% of the signal intensity range: min−0.001″(max-min), and saturation is calculated as the maximum intensity measured on the chip plus 0.1% of the signal intensity range: max+0.001″(max-min). The resulting log it transformed data may then be z-transformed to mean zero and standard deviation 1.

Next, gene expression can be correlated to cancer cell growth inhibition. Growth inhibition data (GI50) of the NCI60 cell lines in the presence of any one of thousands of tested compounds can be obtained from the NCI. The correlation between the log it-transformed expression level of each gene in each cell line and the logarithm of GI50 (the concentration of a given compound that results in a 50% inhibition of growth) can be calculated, e.g., using the Pearson correlation coefficient or the Spearman Rank-Order correlation coefficient. Instead of using GI50s, any other measure of patient sensitivity to a given compound may be correlated to a subject's gene expression levels. Since a plurality of measurements may be available for a single gene, the most accurate determination of correlation coefficient can be, e.g., the median of the correlation coefficients calculated for all probes measuring expression of the same gene.

The median correlation coefficient of gene expression measured on a probe to growth inhibition or patient sensitivity can be calculated for all genes, and genes that have a median correlation above, e.g., 0.3, 0.4, 0.5, 0.6, 0.7, 0.8, 0.9, 0.95, or 0.99 can be used as biomarker genes for assessing responsiveness of a cancer patient (e.g., a lymphoma patient, such as a DLBCL patient) to a treatment for cancer. Preferably, the correlation coefficient of a biomarker gene will exceed 0.3. This can be repeated for multiple drug compounds to be tested. The result is a list of marker genes that correlates to sensitivity for each drug or drug combination tested. Such methods are further described in PCT Publication No. WO 2011/135459, which is incorporated herein in its entirety.

### Methods of Treatment

The diagnostic methods of the invention permit the assessment of whether a given subject is likely to be responsiveness to treatment with a drug or drug combination, and can therefore be used to direct the subject's treatment (e.g., as a first line therapy and/or as a second or third line therapy). Exemplary treatments include combination treatment with CVP, COPE, MBVP, DHAP, ICE, VIM, MIME, ESHAP, ABVD, BEACOPP, CBV, CEPP, COPP, EPOCH, FCM, GemOx, CVAD-A, CVAD-B, MINE, MOPP, CHOP, CHOEP, and/or BeICHOP; and monotherapy with doxorubicin, bendamustine, prednisolone, cyclophosphamide, MTX, vincristine, and/or romidepsin.

A patient found to be responsive to a drug treatment or treatment combination according to the methods of the invention may be preferentially selected for treatment with that specific therapy. Thus, the invention further features methods of treatment that leverage the patient's treatment responsiveness profile to improve outcomes. For example, a patient can be identified as responsive to a drug treatment or treatment combination by determining the expression level of one or more biomarkers in a biological sample obtained from the subject (e.g., hsa-miR-766_st and/or hsa-miR-34b_st or any one or more of the biomarkers or biomarker sets shown in Table 1 corresponding to the indicated therapy), and subsequently administered the treatment or treatments to which the patient is predicted to respond (e.g., alone or as part of a combination treatment). Alternatively, a subject can be, e.g., identified as less responsive to a drug treatment or treatment combination by determining the expression level of one or more biomarkers in a biological sample obtained from the subject (e.g., hsa-miR-766_st and/or hsa-miR-34b_st or any one or more of the biomarkers or biomarker sets shown in Table 1 corresponding to the indicated therapy), and subsequently administered a drug treatment or treatment predicted to have a greater effect than administration of the first drug treatment or treatment combination. In preferred embodiments, the level of expression of one or more biomarkers (e.g., hsa-miR-766_st and/or hsa-miR-34b_st or any one or more of the biomarkers or biomarker sets shown in Table 1 corresponding to the indicated therapy) to a drug treatment or treatment combination is assayed in a sample from a subject. In further embodiments, the level of expression of one or more second biomarkers (e.g., the biomarkers in Table 1) to a drug treatment or treatment combination is assayed in a sample from a subject.

### Combination Treatments

The invention features drug response predictors for drug treatments or combination treatments, in which the expression level of one or more of a panel of biomarkers (e.g., those shown in Table 1) is used to determine a cancer patient's (e.g., a lymphoma patient, such as a DLBCL patient) responsiveness to a drug treatment or combination treatment. A drug treatment or combination treatment can be useful for treating or preventing a condition, disease, or disorder such as those described herein (e.g., lymphoma (e.g., DLBCL)). In certain embodiments, the drug treatment or combination treatment is one previously thought to be ineffective for treating or preventing a condition, disease, or disorder, such as those described herein, in a patient or subpopulation of patients, but which may be effective in certain subject subpopulations (e.g., a subpopulation identifiable by the methods of the invention). Exemplary drug treatments or combination treatments include, but are not limited to, COPE, MBVP, DHAP, ICE, VIM, MIME, ESHAP, CBV, EPOCH, FCM, CVAD-A, CVAD-B, MINE, MOPP, CHOEP, BeICHOP, doxorubicin, bendamustine, ABVD, CHOP, prednisolone, and GemOx, as well as analogs thereof, such as prodrugs, derivatives, metabolites, enantiomers, and combinations thereof.

Once a patient is determined to be responsive to one or more treatments, according to the methods of the invention, one or more of the treatments or combination treatments (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, or BeICHOP, or an analog thereof) can be administered in combination with one or more additional therapies, including but not limited to therapeutic agents (e.g., compounds, pharmaceuticals, or compositions), treatments, therapies, medical procedures, and combinations thereof. Such therapies can also be administered in lieu of the treatment or treatment combination (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, or BeICHOP, or analog thereof), if the subject is, for example, identified as non-responsive to the treatment or treatment combination according to the methods of the invention.

For example, once a cancer patient (e.g., a lymphoma patient, such as a DLBCL patient) has been determined to be responsive to treatment with CVP (e.g., by assessing the expression level of one or more or all of the biomarkers shown in Table 1 for CVP), the cancer patient can be administered the CVP therapy, e.g., according to methods known in the art. Biomarkers that may be used to determine responsiveness to treatment with CVP may include those shown in Table 1, e.g., one or more of hsa-miR-106b-star_st, hsa-miR-1181_st, hsa-miR-25-star_st, hsa-miR-432_st, hsa-miR-551b-star_st, hsa-miR-652_st, hsa-miR-671-5p_st, and/or hsa-miR-93-star_st.

For example, once a cancer patient (e.g., a lymphoma patient, such as a DLBCL patient) has been determined to be responsive to treatment with COPE (e.g., by assessing the expression level of one or more or all of the biomarkers shown in Table 1 for COPE), the cancer patient can be administered the COPE therapy, e.g., according to methods known in the art. Biomarkers that may be used to determine responsiveness to treatment with MBVP may include the biomarkers shown in Table 1, in particular hsa-miR-766_st and/or hsa-miR-34b_st. Additional biomarkers that may used to determine responsiveness to treatment with COPE may include one or more of ACA48_x_st, ENSG00000202498_x_st, HBII-85-26_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and/or hsa-miR-93-star_st.

For example, once a cancer patient (e.g., a lymphoma patient, such as a DLBCL patient) has been determined to be responsive to treatment with MBVP (e.g., by assessing the expression level of one or more or all of the biomarkers shown in Table 1 for MBVP), the cancer patient can be administered the MBVP therapy, e.g., according to methods known in the art. Biomarkers that may be used to determine responsiveness to treatment with MBVP may include the biomarkers shown in Table 1, in particular hsa-miR-766_st and/or hsa-miR-34b_st. Additional biomarkers that may used to determine responsiveness to treatment with MBVP may include one or more of ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and/or hsa-miR-93-star_st.

For example, once a cancer patient (e.g., a lymphoma patient, such as a DLBCL patient) has been determined to be responsive to treatment with MTX (e.g., by assessing the expression level of one or more or all of the biomarkers shown in Table 1 for MTX), the cancer patient can be administered the MTX therapy, e.g., according to methods known in the art. Biomarkers that may be used to determine responsiveness to treatment with MTX may include the biomarkers shown in Table 1, in particular hsa-miR-766_st and/or hsa-miR-34b_st. Additional biomarkers that may used to determine responsiveness to treatment with MTX may include one or more of ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and/or hsa-miR-936_st.

For example, once a cancer patient (e.g., a lymphoma patient, such as a DLBCL patient) has been determined to be responsive to treatment with DHAP (e.g., by assessing the expression level of one or more or all of the biomarkers shown in Table 1 for DHAP), the cancer patient can be administered the DHAP therapy, e.g., according to methods known in the art. Biomarkers that may be used to determine responsiveness to treatment with DHAP may include the biomarkers shown in Table 1, in particular hsa-miR-766_st and/or hsa-miR-34b_st. Additional biomarkers that may used to determine responsiveness to treatment with DHAP may include one or more of ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2_s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and/or hsa-miR-938_st.

For example, once a cancer patient (e.g., a lymphoma patient, such as a DLBCL patient) has been determined to be responsive to treatment with prednisolone (e.g., by assessing the expression level of one or more or all of the biomarkers shown in Table 1 for prednisolone), the cancer patient can be administered the prednisolone therapy, e.g., according to methods known in the art. Biomarkers that may be used to determine responsiveness to treatment with prednisolone may include the biomarkers shown in Table 1, in particular hsa-miR-766_st. Additional biomarkers that may used to determine responsiveness to treatment with prednisolone may include one or more of ACA18_x_st, ACA24_s_st, ACA24_x_st, ACA25_x_st, ACA3-2_st, ACA3_st, ACA40_x_st, ACA47_st, ACA48_st, ACA52_st, ACA54_st, ACA62_st, ACA7_s_st, ENSG00000199411_s_st, ENSG00000200394_st, ENSG00000200879_st, ENSG00000207002_st, HBII-180A_x_st, HBII-202_st, HBII-239_st, HBII-382_s_st, HBII-419_st, HBII-429_st, HBII-52-32_x_st, HBII-55_st, U104_st, U17a_st, U17b_st, U17b_x_st, U25_st, U26_st, U27_st, U28_st, U29_st, U3-2_s_st, U30_st, U31_st, U31_x_st, U33_st, U35A_st, U36A_x_st, U38A_st, U38B_st, U41_st, U46_st, U46_x_st, U49A_st, U50B_st, U50B_x_st, U50_st, U55_st, U56_st, U56_x_st, U57_st, U67_st, U67_x_st, U68_st, U68_x_st, U70_x_st, U73a_st, U78_s_st, U78_x_st, U83_st, U93_st, U95_st, hsa-miR-1181_st, hsa-miR-1207-5p_st, hsa-miR-1246_st, hsa-miR-1268_st, hsa-miR-1275_st, hsa-miR-17-star_st, hsa-miR-17_st, hsa-miR-195-star_st, hsa-miR-19b_st, hsa-miR-297_st, hsa-miR-571_st, hsa-miR-768-3p_st, hsa-miR-768-5p_st, hsa-miR-877-star_st, hsa-miR-92a-1-star_st, hsa-miR-92a-2-star_st, hsa-miR-92a_st, hsa-miR-938_st, and/or hsa-miR-939_st.

For example, once a cancer patient (e.g., a lymphoma patient, such as a DLBCL patient) has been determined to be responsive to treatment with cyclophosphamide (e.g., by assessing the expression level of one or more or all of the biomarkers shown in Table 1 for cyclophosphamide), the cancer patient can be administered the cyclophosphamide therapy, e.g., according to methods known in the art. Biomarkers that may be used to determine responsiveness to treatment with cyclophosphamide may include those shown in Table 1, e.g., one or more of ENSG00000200879_st, ENSG00000202252_st, HBII-142_st, HBII-142_x_st, HBII-202_st, U42A_st, U59B_st, hsa-miR-1202_st, hsa-miR-184_st, hsa-miR-187_st, hsa-miR-191_st, hsa-miR-196a_st, hsa-miR-203_st, hsa-miR-29b-2-star_st, hsa-miR-29c-star_st, hsa-miR-328_st, hsa-miR-375_st, hsa-miR-423-3p_st, hsa-miR-423-5p_st, hsa-miR-449a_st, hsa-miR-449b_st, hsa-miR-489_st, and/or hsa-miR-768-3p_st.

For example, once a cancer patient (e.g., a lymphoma patient, such as a DLBCL patient) has been determined to be responsive to treatment with doxorubicin (e.g., by assessing the expression level of one or more or all of the biomarkers shown in Table 1 for doxorubicin), the cancer patient can be administered the doxorubicin therapy, e.g., according to methods known in the art. Biomarkers that may be used to determine responsiveness to treatment with doxorubicin may include the biomarkers shown in Table 1, in particular hsa-miR-766_st and/or hsa-miR-34b_st. Additional biomarkers that may used to determine responsiveness to treatment with doxorubicin may include one or more of ACA13_st, ACA48_x_st, U104_st, U55_st, U55_x_st, U74_x_st, hsa-miR-106a-star_st, hsa-miR-106b-star_st, hsa-miR-106b_st, hsa-miR-124_st, hsa-miR-1281_st, hsa-miR-1299_st, hsa-miR-140-3p_st, hsa-miR-195-star_st, hsa-miR-297_st, hsa-miR-29b-2-star_st, hsa-miR-33b-star_st, hsa-miR-342-3p_st, hsa-miR-342-5p_st, hsa-miR-432_st, hsa-miR-550-star_st, hsa-miR-629-star_st, hsa-miR-629_st, hsa-miR-652_st, hsa-miR-654-3p_st, hsa-miR-671-5p_st, hsa-miR-768-3p_st, hsa-miR-877-star_st, hsa-miR-93-star_st, and/or hsa-miR-93_st.

For example, once a cancer patient (e.g., a lymphoma patient, such as a DLBCL patient) has been determined to be responsive to treatment with vincristine (e.g., by assessing the expression level of one or more or all of the biomarkers shown in Table 1 for vincristine), the cancer patient can be administered the vincristine therapy, e.g., according to methods known in the art. Biomarkers that may be used to determine responsiveness to treatment with vincristine may include those shown in Table 1, e.g., one or more of hsa-miR-106b-star_st, hsa-miR-25-star_st, hsa-miR-432_st, hsa-miR-551b-star_st, hsa-miR-671-5p_st, and/or hsa-miR-93-star_st.

For example, once a cancer patient (e.g., a lymphoma patient, such as a DLBCL patient) has been determined to be responsive to treatment with ICE (e.g., by assessing the expression level of one or more or all of the biomarkers shown in Table 1 for ICE), the cancer patient can be administered the ICE therapy, e.g., according to methods known in the art. Biomarkers that may be used to determine responsiveness to treatment with ICE may include the biomarkers shown in Table 1, in particular hsa-miR-766_st and/or hsa-miR-34b_st. Additional biomarkers that may used to determine responsiveness to treatment with ICE may include one or more of 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and/or hsa-miR-938_st.

For example, once a cancer patient (e.g., a lymphoma patient, such as a DLBCL patient) has been determined to be responsive to treatment with VIM (e.g., by assessing the expression level of one or more or all of the biomarkers shown in Table 1 for VIM), the cancer patient can be administered the VIM therapy, e.g., according to methods known in the art. Biomarkers that may be used to determine responsiveness to treatment with VIM may include the biomarkers shown in Table 1, in particular hsa-miR-766_st and/or hsa-miR-34b_st. Additional biomarkers that may used to determine responsiveness to treatment with VIM may include one or more of ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, once a cancer patient (e.g., a lymphoma patient, such as a DLBCL patient) has been determined to be responsive to treatment with MIME (e.g., by assessing the expression level of one or more or all of the biomarkers shown in Table 1 for MIME), the cancer patient can be administered the MIME therapy, e.g., according to methods known in the art. Biomarkers that may be used to determine responsiveness to treatment with MIME may include the biomarkers shown in Table 1, in particular hsa-miR-766_st and/or hsa-miR-34b_st. Additional biomarkers that may used to determine responsiveness to treatment with MIME may include one or more of ACA10_s_st, ACA11_st, ACA13_st, ACA18_x_st, ACA21_st, ACA40_x_st, ACA48_x_st, ACA51_x_st, ACA55_st, ACA61_st, ACA7_s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and/or hsa-miR-93-star_st.

For example, once a cancer patient (e.g., a lymphoma patient, such as a DLBCL patient) has been determined to be responsive to treatment with bendamustine (e.g., by assessing the expression level of one or more or all of the biomarkers shown in Table 1 for bendamustine), the cancer patient can be administered the bendamustine therapy, e.g., according to methods known in the art. Biomarkers that may be used to determine responsiveness to treatment with bendamustine may include the biomarkers shown in Table 1, in particular hsa-miR-766_st and/or hsa-miR-34b_st. Additional biomarkers that may used to determine responsiveness to treatment with bendamustine may include one or more of ACA11_st, ACA24_x_st, ACA7_s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and/or hsa-miR-93-star_st.

For example, once a cancer patient (e.g., a lymphoma patient, such as a DLBCL patient) has been determined to be responsive to treatment with ESHAP (e.g., by assessing the expression level of one or more or all of the biomarkers shown in Table 1 for ESHAP), the cancer patient can be administered the ESHAP therapy, e.g., according to methods known in the art. Biomarkers that may be used to determine responsiveness to treatment with ESHAP may include the biomarkers shown in Table 1, in particular hsa-miR-766_st and/or hsa-miR-34b_st. Additional biomarkers that may used to determine responsiveness to treatment with ESHAP may include one or more of ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and/or hsa-miR-768-5p_st.

For example, once a cancer patient (e.g., a lymphoma patient, such as a DLBCL patient) has been determine to be responsive to treatment with ABVD (e.g., by assessing the expression level of one or more or all of the biomarkers shown in Table 1 for ABVD), the cancer patient can be administered the ABVD therapy, e.g., according to methods known in the art. Biomarkers that may be used to determine responsiveness to treatment with ABVD may include the biomarkers shown in Table 1, in particular hsa-miR-766_st. Additional biomarkers that may used to determine responsiveness to treatment with ABVD may include one or more of 14qII-14_st, 14qII-14_x_st, 14qII-1_st, 14qII-1_x_st, 14qII-22_x_st, 14qII-26_st, 14qII-26_x_st, HBII-85-26_st, HBII-85-6_x_st, U22_st, U48_st, U55_st, U55_x_st, hsa-miR-124_st, hsa-miR-125b-1-star_st, hsa-miR-127-3p_st, hsa-miR-1271_st, hsa-miR-1281_st, hsa-miR-1299_st, hsa-miR-134_st, hsa-miR-140-3p_st, hsa-miR-143_st, hsa-miR-145_st, hsa-miR-154_st, hsa-miR-195-star_st, hsa-miR-199a-5p_st, hsa-miR-199b-3p_st, hsa-miR-299-3p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-495_st, hsa-miR-629-star_st, hsa-miR-654-3p_st, hsa-miR-665_st, hsa-miR-671-5p_st, hsa-miR-758_st, and/or hsa-miR-923_st.

For example, once a cancer patient (e.g., a lymphoma patient, such as a DLBCL patient) has been determined to be responsive to treatment with BEACOPP (e.g., by assessing the expression level of one or more or all of the biomarkers shown in Table 1 for BEACOPP), the cancer patient can be administered the BEACOPP therapy, e.g., according to methods known in the art. Biomarkers that may be used to determine responsiveness to treatment with BEACOPP may include those shown in Table 1, e.g., one or more of 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-85-26_st, HBII-85-6_x_st, hsa-miR-106b-star_st, hsa-miR-124_st, hsa-miR-127-3p_st, hsa-miR-127-5p_st, hsa-miR-1281_st, hsa-miR-1299_st, hsa-miR-134_st, hsa-miR-140-3p_st, hsa-miR-154_st, hsa-miR-181a-star_st, hsa-miR-195-star_st, hsa-miR-199a-5p_st, hsa-miR-214-star_st, hsa-miR-299-3p_st, hsa-miR-337-5p_st, hsa-miR-339-5p_st, hsa-miR-342-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-410_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-615-3p_st, hsa-miR-654-3p_st, hsa-miR-665_st, hsa-miR-671-5p_st, hsa-miR-768-3p_st, and/or hsa-miR-92b_st.

For example, once a cancer patient (e.g., a lymphoma patient, such as a DLBCL patient) has been determined to be responsive to treatment with CBV (e.g., by assessing the expression level of one or more or all of the biomarkers shown in Table 1 for CBV), the cancer patient can be administered the CBV therapy, e.g., according to methods known in the art. Biomarkers that may be used to determine responsiveness to treatment with CBV may include the biomarkers shown in Table 1, in particular hsa-miR-766_st and/or hsa-miR-34b_st. Additional biomarkers that may used to determine responsiveness to treatment with CBV may include one or more of hsa-miR-34b_st, ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and/or hsa-miR-938_st.

For example, once a cancer patient (e.g., a lymphoma patient, such as a DLBCL patient) has been determined to be responsive to treatment with CEPP (e.g., by assessing the expression level of one or more or all of the biomarkers shown in Table 1 for CEPP), the cancer patient can be administered the CEPP therapy, e.g., according to methods known in the art. Biomarkers that may be used to determine responsiveness to treatment with CEPP may include those shown in Table 1, e.g., one or more of 14qII-12_st, 14qII-14_st, 14qII-14_x_st, 14qII-1_st, 14qII-1_x_st, 14qII-22_x_st, 14qII-26_st, 14qII-26_x_st, 14qII-3_st, ENSG00000202498_x_st, HBII-336_st, HBII-85-26_st, HBII-85-6_x_st, hsa-miR-125b-1-star_st, hsa-miR-127-3p_st, hsa-miR-127-5p_st, hsa-miR-1271_st, hsa-miR-1281_st, hsa-miR-134_st, hsa-miR-140-3p_st, hsa-miR-143-star_st, hsa-miR-143_st, hsa-miR-145_st, hsa-miR-154_st, hsa-miR-155_st, hsa-miR-181a-star_st, hsa-miR-181a_st, hsa-miR-181b_st, hsa-miR-181c_st, hsa-miR-199a-5p_st, hsa-miR-199b-3p_st, hsa-miR-21-star_st, hsa-miR-214-star_st, hsa-miR-27a_st, hsa-miR-299-3p_st, hsa-miR-337-5p_st, hsa-miR-339-5p_st, hsa-miR-342-3p_st, hsa-miR-34c-3p_st, hsa-miR-370_st, hsa-miR-376c_st, hsa-miR-377-star_st, hsa-miR-379_st, hsa-miR-381_st, hsa-miR-382_st, hsa-miR-409-3p_st, hsa-miR-409-5p_st, hsa-miR-411_st, hsa-miR-431_st, hsa-miR-432_st, hsa-miR-433_st, hsa-miR-485-3p_st, hsa-miR-485-5p_st, hsa-miR-487a_st, hsa-miR-487b_st, hsa-miR-493_st, hsa-miR-494_st, hsa-miR-543_st, hsa-miR-574-3p_st, hsa-miR-615-3p_st, hsa-miR-654-3p_st, hsa-miR-665_st, hsa-miR-671-5p_st, hsa-miR-758_st, and/or hsa-miR-92b_st.

For example, once a cancer patient (e.g., a lymphoma patient, such as a DLBCL patient) has been determined to be responsive to treatment with COPP (e.g., by assessing the expression level of one or more or all of the biomarkers shown in Table 1 for COPP), the cancer patient can be administered the COPP therapy, e.g., according to methods known in the art. Biomarkers that may be used to determine responsiveness to treatment with COPP may include those shown in Table 1, e.g., one or more of hsa-miR-106b-star_st, hsa-miR-1181_st, hsa-miR-432_st, hsa-miR-551b-star_st, hsa-miR-652_st, hsa-miR-671-5p_st, hsa-miR-93-star_st, and/or hsa-miR-93_st.

For example, once a cancer patient (e.g., a lymphoma patient, such as a DLBCL patient) has been determined to be responsive to treatment with EPOCH (e.g., by assessing the expression level of one or more or all of the biomarkers shown in Table 1 for EPOCH), the cancer patient can be administered the EPOCH therapy, e.g., according to methods known in the art. Biomarkers that may be used to determine responsiveness to treatment with EPOCH may include the biomarkers shown in Table 1, in particular hsa-miR-766_st and/or hsa-miR-34b_st. Additional biomarkers that may used to determine responsiveness to treatment with EPOCH may include one or more of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93_st.

For example, once a cancer patient (e.g., a lymphoma patient, such as a DLBCL patient) has been determined to be responsive to treatment with FCM (e.g., by assessing the expression level of one or more or all of the biomarkers shown in Table 1 for FCM), the cancer patient can be administered the FCM therapy, e.g., according to methods known in the art. Biomarkers that may be used to determine responsiveness to treatment with FCM may include the biomarkers shown in Table 1, in particular hsa-miR-766_st and/or hsa-miR-34b_st. Additional biomarkers that may used to determine responsiveness to treatment with FCM may include one or more of ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181 b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and/or hsa-miR-92b_st.

For example, once a cancer patient (e.g., a lymphoma patient, such as a DLBCL patient) has been determined to be responsive to treatment with GemOx (e.g., by assessing the expression level of one or more or all of the biomarkers shown in Table 1 for GemOx), the cancer patient can be administered the GemOx therapy, e.g., according to methods known in the art. Biomarkers that may be used to determine responsiveness to treatment with GemOx may include the biomarkers shown in Table 1, in particular hsa-miR-34b_st. Additional biomarkers that may used to determine responsiveness to treatment with GemOx may include one or more of hsa-miR-126_st, hsa-miR-153_st, hsa-miR-19b_st, hsa-miR-424-star_st, hsa-miR-491-3p_st, and/or hsa-miR-503_st.

For example, once a cancer patient (e.g., a lymphoma patient, such as a DLBCL patient) has been determined to be responsive to treatment with CVAD-A (e.g., by assessing the expression level of one or more or all of the biomarkers shown in Table 1 for CVAD-A), the cancer patient can be administered the CVAD-A therapy, e.g., according to methods known in the art. Biomarkers that may be used to determine responsiveness to treatment with CVAD-A may include the biomarkers shown in Table 1, in particular hsa-miR-766_st and/or hsa-miR-34b_st. Additional biomarkers that may used to determine responsiveness to treatment with CVAD-A may include one or more of ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and/or hsa-miR-92a-2-star_st.

For example, once a cancer patient (e.g., a lymphoma patient, such as a DLBCL patient) has been determined to be responsive to treatment with CVAD-B (e.g., by assessing the expression level of one or more or all of the biomarkers shown in Table 1 for CVAD-B), the cancer patient can be administered the CVAD-B therapy, e.g., according to methods known in the art. Biomarkers that may be used to determine responsiveness to treatment with CVAD-B may include the biomarkers shown in Table 1, in particular hsa-miR-766_st and/or hsa-miR-34b_st. Additional biomarkers that may used to determine responsiveness to treatment with CVAD-B may include one or more of hsa-miR-766_st, hsa-miR-34b_st, ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_st, hsa-miR-663b_st, hsa-miR-768-5p_st, and/or hsa-miR-92a_st.

For example, once a cancer patient (e.g., a lymphoma patient, such as a DLBCL patient) has been determined to be responsive to treatment with MINE (e.g., by assessing the expression level of one or more or all of the biomarkers shown in Table 1 for MINE), the cancer patient can be administered the MINE therapy, e.g., according to methods known in the art. Biomarkers that may be used to determine responsiveness to treatment with MINE may include the biomarkers shown in Table 1, in particular hsa-miR-766_st and/or hsa-miR-34b_st. Additional biomarkers that may used to determine responsiveness to treatment with MINE may include one or more of 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and/or hsa-miR-938_st.

For example, once a cancer patient (e.g., a lymphoma patient, such as a DLBCL patient) has been determine to be responsive to treatment with MOPP (e.g., by assessing the expression level of one or more or all of the biomarkers shown in Table 1 for MOPP), the cancer patient can be administered the MOPP therapy, e.g., according to methods known in the art. Biomarkers that may be used to determine responsiveness to treatment with MOPP may include the biomarkers shown in Table 1, in particular hsa-miR-766_st and/or hsa-miR-34b_st. Additional biomarkers that may used to determine responsiveness to treatment with MOPP may include one or more of ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and/or hsa-miR-92b_st.

For example, once a cancer patient (e.g., a lymphoma patient, such as a DLBCL patient) has been determine to be responsive to treatment with romidepsin (e.g., by assessing the expression level of one or more or all of the biomarkers shown in Table 1 for romidepsin), the cancer patient can be administered the romidepsin therapy, e.g., according to methods known in the art. Biomarkers that may be used to determine responsiveness to treatment with romidepsin may include those shown in Table 1, e.g., one or more of hsa-miR-105_st, hsa-miR-106b_st, hsa-miR-185_st, hsa-miR-188-5p_st, hsa-miR-25_st, hsa-miR-320c_st, hsa-miR-362-5p_st, hsa-miR-500-star_st, hsa-miR-500_st, hsa-miR-501-3p_st, hsa-miR-501-5p_st, hsa-miR-502-3p_st, hsa-miR-532-3p_st, hsa-miR-532-5p_st, hsa-miR-584_st, hsa-miR-611_st, hsa-miR-652_st, hsa-miR-660_st, hsa-miR-767-5p_st, hsa-miR-769-3p_st, hsa-miR-877_st, and/or hsa-miR-93_st

For example, once a cancer patient (e.g., a lymphoma patient, such as a DLBCL patient) has been determine to be responsive to treatment with CHOP (e.g., by assessing the expression level of one or more or all of the biomarkers shown in Table 1 for CHOP), the cancer patient can be administered the CHOP therapy, e.g., according to methods known in the art. Biomarkers that may be used to determine responsiveness to treatment with CHOP may include the biomarkers shown in Table 1, in particular hsa-miR-766_st. Additional biomarkers that may used to determine responsiveness to treatment with CHOP may include one or more of ACA48_x_st, U55_x_st, hsa-miR-106b-star_st, hsa-miR-106b_st, hsa-miR-1181_st, hsa-miR-124_st, hsa-miR-1299_st, hsa-miR-25-star_st, hsa-miR-33b-star_st, hsa-miR-432_st, hsa-miR-551b-star_st, hsa-miR-629-star_st, hsa-miR-629_st, hsa-miR-652_st, hsa-miR-654-3p_st, hsa-miR-671-5p_st, hsa-miR-877-star_st, hsa-miR-93-star_st, and/or hsa-miR-93_st.

For example, once a cancer patient (e.g., a lymphoma patient, such as a DLBCL patient) has been determine to be responsive to treatment with CHOEP (e.g., by assessing the expression level of one or more or all of the biomarkers shown in Table 1 for CHOEP), the cancer patient can be administered the CHOEP therapy, e.g., according to methods known in the art. Biomarkers that may be used to determine responsiveness to treatment with CHOEP may include the biomarkers shown in Table 1, in particular hsa-miR-766_st and/or hsa-miR-34b_st. Additional biomarkers that may used to determine responsiveness to treatment with CHOEP may include one or more of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and/or hsa-miR-93 st.

For example, once a cancer patient (e.g., a lymphoma patient, such as a DLBCL patient) has been determine to be responsive to treatment with BeICHOP (e.g., by assessing the expression level of one or more or all of the biomarkers shown in Table 1 for BeICHOP), the cancer patient can be administered the BeICHOP therapy, e.g., according to methods known in the art. Biomarkers that may be used to determine responsiveness to treatment with BeICHOP may include the biomarkers shown in Table 1, in particular hsa-miR-766_st and/or hsa-miR-34b_st. Additional biomarkers that may used to determine responsiveness to treatment with BeICHOP may include one or more of ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and/or hsa-miR-93_st.

Methods of the invention can be used to determine the responsiveness of a subject to combination treatments. In such combinations, drug treatments of the invention may be formulated with or administered concurrently with, prior to, or subsequent to, one or more other desired treatments. The particular combination of treatments to employ in a combination regimen will take into account compatibility of the desired therapeutics and/or procedures and the desired therapeutic effect to be achieved. It will also be appreciated that the treatments employed may achieve a desired effect for a condition, disease, or disorder, or they may achieve different effects (e.g., control of any adverse effects).

In general, for use in therapy, the treatments described herein may be used alone, as mixtures of two or more agents, or in combination with other agents, compounds, and/or pharmaceuticals. Examples of other agents that can be combined with the compounds described herein include agents that are known to be used for the treatment of lymphoma (e.g., DLBCL)). Other potential agents to combine with the compounds described herein can include agents for the treatment of different yet associated or related symptoms or indications. Depending on the mode of administration, the agents may be formulated into suitable compositions to permit facile delivery. Each component of a combination therapy may be formulated in a variety of ways that are known in the art. For example, one or more of the agents of the combination therapy may be formulated together or separately. The target drug or drug combination of the invention and any additional agents may be suitably administered to the patient at one time or over a series of treatments.

The combination treatments described herein may provide “synergy” and exhibit a “synergistic” effect, i.e., an effect achieved when the active ingredient(s) used together is greater than the sum of the effects that results from using the ingredients separately. A synergistic effect may be attained when the active ingredients are: (1) co-formulated and administered or delivered simultaneously in a combined, unit dosage formulation; (2) delivered by alternating therapies, or in parallel administrations, as separate formulations; or (3) by some other regimen. When delivered in alternating therapy, a synergistic effect may be attained when the compounds, agents, and/or treatments are administered or delivered sequentially, e.g., by different injections in separate syringes. Suitable dosages may be lowered due to the combined action (synergy) of a treatment and other co-administered agents or treatments.

Each component of a combination treatment, as described herein, may be formulated in a variety of ways that are known in the art. For example, one or more of the combination therapy may be formulated together or separately. The individually or separately formulated agents can be packaged together as a kit. Non-limiting examples include, but are not limited to, kits that contain, e.g., a liquid in a container (e.g., a bag or a vial), two pills, a pill and a powder, a suppository, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like. Two or more components may be mixed together in a liquid, tablet, capsule, or other vehicle, or may be partitioned. Typically, one or more of the therapies indicated in Table 1 are administered intravenously.

Exemplary therapies that can be combined with drugs or drug combinations of the invention can include, for example, other known chemotherapeutic agents and/or non-pharmacological therapies (e.g., surgery or radiation therapy). Chemotherapeutic agents suitable for administration in combination with treatments of the invention (in particular, COPE, MBVP, DHAP, ICE, VIM, MIME, ESHAP, CBV, EPOCH, FCM, CVAD-A, CVAD-B, MINE, MOPP, CHOEP, BeICHOP, doxorubicin, bendamustine, ABVD, CHOP, prednisolone, and GemOx) may include, for example, one or more of the following: cyclophosphamide, vincristine, etoposide, methotrexate, carmustine, dexamethasone, ara-C, cisplatin, dacarbazine, procarbazine, oxaliplatin, cytarabine, 5-flourouracil, capecitabine, tegafur, irinotecan, oxaliplatin, cetuximab, leucovorin, SN-38, everolimus, temsirolimus, bleomycin, lomustine, depsipeptide, carboplatin, bortezomib, erlotinib, gemcitabine, mitoxantrone, busulfan, epirubicin, arsenic trioxide, bendamustine, fulvestrant, teniposide, adriamycin, decitabine, estramustine, azaguanine, aclarubicin, mitoxantrone, mitomycin, paclitaxel, taxotere, dexamethasone, prednisolone, prednisone, methylprednisolone, methyl-gag, belinostat, carboplatin, idarubicin, melphalan, IL4-PR38, valproic acid, all-trans retinoic acid (ATRA), cytoxan, topotecan, suberoylanilide hydroxamic acid (SAHA, vorinostat), leukeran, fludarabine, vinblastine, dacarbazine, hydroxyurea, tegafur, daunorubicin, mechlorethamine, streptozocin, mercaptopurine, dactinomycin, tretinoin, ifosfamide, tamoxifen, floxuridine, thioguanine, PSC 833, herceptin, bevacizumab, celecoxib, iressa, anastrozole, letrozole, rituximab, radiation, and histone deacetylase (HDAC) inhibitors.

### Administration of Target Drugs

Routes of administration, frequency of administration, and/or dosage of combination treatments (e.g., COPE, MBVP, DHAP, ICE, VIM, MIME, ESHAP, CBV, EPOCH, FCM, CVAD-A, CVAD-B, MINE, MOPP, CHOEP, BeICHOP, ABVD, CHOP, and GemOx) or monotherapies (e.g., doxorubicin, bendamustine, and prednisolone) may vary from individual to individual, and may be readily established using standard techniques. Once a patient has been determined to be responsive to one or more treatments, according to the methods described herein, the treatment(s) of the invention can be administered to the patient by the usual means known in the art, for example, by injection, intravenously, orally, subcutaneously, intraperitoneally, intramuscularly, by infusion, by infiltration, by irrigation, intra-articularly, by inhalation, topically, rectally, vaginally, cutaneously, nasally, transdermally, or by ocular administration and the like. In certain embodiments, the treatment or combination treatment is administered intravenously. In particular embodiments, COPE, MBVP, DHAP, ICE, VIM, MIME, ESHAP, CBV, EPOCH, FCM, CVAD-A, CVAD-B, MINE, MOPP, CHOEP, BeICHOP, doxorubicin, bendamustine, ABVD, CHOP, prednisolone, and/or GemOx are administered by intravenous injection. For administration by injection, infiltration, or infusion, a target drug may be suspended or dissolved, as known in the art, in a vehicle suitable for injection, infiltration, or infusion. Such vehicles include, e.g., isotonic saline, buffered or unbuffered, and the like. Depending on the intended use, they also may contain other ingredients, including other active ingredients, such as isotonicity agents, sodium chloride, pH modifiers, colorants, preservatives, antibodies, enzymes, antibiotics, antifungals, antivirals, other anti-infective agents, and/or diagnostic aids such as radio-opaque dyes, radiolabeled agents, and the like, as known in the art. Compositions including a treatment or combination treatment may include a simple solution or suspension of the target drug or a pharmaceutically acceptable salt of the target drug, in distilled water or saline. Alternatively, a treatment or combination treatment may be delivered by other means, such as intranasally, by inhalation, or in the form of liposomes, nanocapsules, vesicles, and the like. Compositions for intranasal administration usually take the form of drops, sprays containing liquid forms (solutions, suspensions, emulsions, liposomes, etc.) of the active compounds. Administration by inhalation generally involves formation of vapors, mists, dry powders or aerosols, and again may include solutions, suspensions, emulsions and the like containing the active therapeutic agents.

Administration of a treatment or combination treatment of the invention to a patient can be performed according to, e.g., courses of therapy standard in the art. A treatment or combination treatment can be administered at a frequency of, for example, at least once hourly, once daily, twice daily, once weekly, once every two weeks, once every three weeks, once every four weeks, once monthly, once every two months, once every three months, once every six months, or once every year. The administration can be repeated at such a frequency for a certain period of time, followed by a period without treatment. In certain embodiments, a treatment or combination treatment is administered once daily for up to five days. In particular embodiments, the administration is repeated every 30 days or every month. Such repeated administrations can occur over a course of therapy lasting a specified length of time (e.g., at least 1 week, 2 weeks, 3 weeks, 1 month, 2 months, 3 months, 6 months, 8 months, 10 months, 12 months, 18 months, 24 months, 36 months, 48 months, or 60 months). In certain embodiments, administration of a target drug is repeated for at least 12 months or 60 months.

A cancer patient (e.g., a lymphoma patient, such as a DLBCL patient) may be monitored or assessed according to the methods of the invention before, during, or after treatment (e.g., a first, second, and/or third line treatment). For example, a patient may be assessed for target drug responsiveness between administrations of the treatment or combination treatment. Multiple assessments can be performed over the course of therapy, and the results combined to determine change in responsiveness of the patient to the treatment or combination treatment over time. Each of the assessments can involve determining the expression level of the same biomarkers of responsiveness (see, e.g., the biomarkers of Table 1), or can involve determining the expression level of different biomarkers (e.g., selecting one or more distinct biomarkers per assessment, or varying only a subset of the biomarkers tested between individual assessments).

Therapeutic and/or prophylactic effect (e.g., benefit) can be monitored by establishing an improved clinical outcome (e.g., more frequent remissions, complete or partial, longer disease-free survival, decreased morbidity, or an improvement in one or more standard tests or assays known in the art for the assessment of the disease, condition, or disorder status) in treated patients (e.g., patients with lymphoma (e.g, patients with DLBCL)) as compared to non-treated patients.

Drug treatments, such as those described herein, may be used alone or in combination with one or more additional therapeutic agents suitable for treatment of a particular indication. For example, a treatment or combination treatment of the invention may be co-administered to a patient who has, or is at risk for developing, lymphoma (e.g., DLBCL) with conventional anti-cancer therapeutic regimens such as surgery, irradiation, chemotherapy and/or bone marrow transplantation (autologous, syngeneic, allogeneic, or unrelated) or in combination with conventional therapeutic regimens to treat age-related cataracts, spinal injuries, wounds or congenital defects. Such combination therapies are described in further detail below.

Patients that can be treated with a treatment or combination treatment as described herein, and/or the pharmaceutically acceptable salts, prodrugs, enantiomers, and metabolites of such compounds, according to the methods of the invention include, for example, patients that have been diagnosed as having any of the diseases or disorders described herein (e.g., lymphoma (e.g., DLBCL)). As used herein, a “patient” or a “subject” refers to any animal (e.g., a mammal), preferably a human. The responsiveness of a patient to administration of a treatment or combination treatment can be determined using the methods of the invention, e.g., by measuring the expression level of one or more biomarkers of responsiveness to the treatment (e.g., doxorubicin, bendamustine, and/or prednisolone) or combination treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, ABVD, CHOP, and/or GemOx, see, e.g., Table 1). A patient may be suffering from, in remission from, or susceptible to developing lymphoma (e.g., DLBCL). Accordingly, pharmaceutical compositions including a target drug may be used to prevent the development or progression of a cancer or other disorder, or delay its appearance or reappearance, or to treat a patient afflicted with a cancer. A lymphoma or other disorder (e.g., DLBCL) treatable by the methods of the invention may be diagnosed using the diagnostic methods described herein and/or by criteria generally accepted in the art. In the case of lymphoma, treatments or combination treatments may be administered either prior to or following surgical removal of primary or other tumors and/or treatment, such as administration of radiotherapy or conventional chemotherapeutic drugs, or bone marrow transplantation (autologous, allogeneic, or syngeneic).

### Biomarker Expression Analysis Methods, Devices, and Kits

The methods, devices, and kits of the invention can be used to predict the responsiveness of a cancer patient (e.g., a lymphoma patient, such as a DLBCL patient) to a treatment or combination treatment of interest based on the expression levels (e.g., mRNA expression level or protein expression level) of one or more biomarkers (e.g., those listed in Table 1) in a biological sample obtained from the patient. The biological sample can include, for example, cells, tissue (e.g., a tissue sample obtained by biopsy), blood, serum, plasma, urine, sputum, cerebrospinal fluid, lymph tissue or fluid, or pancreatic fluid. In certain embodiments, the biological sample is fresh frozen or formalin-fixed paraffin embedded (FFPE) tissue obtained from the patient, such as a tumor sample (e.g., a biopsy). Numerous methods of determining biomarker expression levels, or expression profiling, are known in the art, including, but not limited to, polymerase chain reaction (PCR), reverse transcriptase PCR (RT-PCR), quantitative real-time PCR (qPCR), Northern blots, Western blots, Southern blots, microarrays, NanoString nCounter technologies (e.g., those described in U.S. Patent Application Nos. US 2011/0201515, US 2011/0229888, and US 2013/0017971, each of which is incorporated by reference in its entirety), next generation sequencing (e.g., RNA-Seq techniques), and proteomic techniques (e.g., mass spectrometry or protein arrays).

One or more of the biomarkers (e.g., hsa-miR-766_st, hsa-miR-34b_st, and/or one or more of the other biomarkers shown in Table 1) may be measured in a biological sample obtained from the cancer patient (e.g., a patient suffering from or susceptible to a disease, disorder, or condition treatable with the drugs or combinations of interest, such as lymphoma, e.g., DLBCL) using, e.g., a microarray, sequencing, NanoString, a protein array, or PCR-based methods, such as those described herein. When a microarray is used to assess expression levels of one or more of the biomarkers (e.g., those shown in Table 1), the microarray includes probes capable of recognizing the one or more biomarkers, such as probes of at least about 15 nt in length that have, e.g., a sequence with at least 5 (e.g., at least 10) contiguous nucleotides that are complementary to or identical to the sequence(s) of the biomarker(s). Any one of the biomarkers from Table 1 can be used to assess a patient's responsive (e.g., a patient with DLBCL) to the treatments and combination treatments described herein (e.g., those monotherapies and combination therapies listed in Table 1). Alternatively, a combination of any of the biomarkers of Table 1 for the indicated therapy may be used. For example, combinations that include 5, 10, 15, 20, or more of the biomarkers from Table 1 for each respective treatment may be used.

### PCR-Based Techniques

Tissue or cell samples from mammals can be conveniently assayed for mRNA levels using Northern, dot blot or PCR analysis. For example, qPCR assays are well known in the art. In some embodiments, an mRNA corresponding to a biomarker of sensitivity can be detected in a biological sample by (a) producing cDNA from the sample by reverse transcription using at least one primer; (b) amplifying the cDNA so produced using a target polynucleotide as sense and antisense primers to amplify target cDNAs therein; and (c) detecting the presence of the amplified target cDNA using polynucleotide probes. The primers and probes comprising the sequences described herein (listed in Table 2) may be used to detect expression of one or more of the indicated biomarkers, as described herein. In addition, such methods can include one or more steps that allow one to determine the levels of target mRNA in a biological sample (e.g., by simultaneously examining the levels a comparative control mRNA sequence of a “housekeeping” gene such as an actin family member or GAPDH). Optionally, the sequence of the amplified target cDNA can be determined. The primers for these assays may be labeled for detection according to methods known in the art.

### Microarrays

Expression levels of the biomarkers (e.g., the biomarkers listed in Table 1 (e.g., hsa-miR-766_st and hsa-miR-34b_st)) of the invention may be determined using high-throughput expression profiling platforms, such as microarrays. Microarray technology offers high resolution quantification of mRNA expression levels at a genomic or transcriptomic scale. In certain embodiments, a microarray for use in the methods of the invention for assessing the responsiveness of a cancer patient (e.g., a lymphoma patient, e.g., a DLBCL patient) to a treatment or combination treatment (e.g., COPE, MBVP, DHAP, ICE, VIM, MIME, ESHAP, CBV, EPOCH, FCM, CVAD-A, CVAD-B, MINE, MOPP, CHOEP, BeICHOP, doxorubicin, bendamustine, ABVD, CHOP, prednisolone, and/or GemOx, as well as analogs thereof, such as prodrugs, derivatives, metabolites, enantiomers, and combinations thereof) contains or is produced by generating oligonucleotide probes (e.g., DNA, cDNA, or RNA probes) capable of hybridizing to one or more biomarkers of interest (e.g., one or more of the biomarkers of Tables 1-3) or their complement sequences (preferably the probe sequence is complementary to all or a portion of the biomarker sequence). For example, a probe may have substantial sequence identity to (or share at least 85%, 90%, 95%, 99%, or 100% identity to) all or a portion of a biomarker or its complement sequence, such as hsa-miR-766_st (SEQ ID NO. 246) and hsa-miR-34b_st (SEQ ID NO. 185).

Microarrays of the invention for determining drug or drug combination responsiveness can include probes for one or more (e.g., at least 5, 10, 15, or 20 or more (e.g., all)) COPE biomarkers, one or more (e.g., at least 5, 10, 15, or 20 or more (e.g., all)) MBVP biomarkers, one or more (e.g., at least 5, 10, 15, or 20 or more (e.g., all)) DHAP biomarkers, one or more (e.g., at least 5, 10, 15, or 20 or more (e.g., all)) ICE biomarkers, one or more (e.g., at least 5, 10, 15, or 20 or more (e.g., all)) VIM biomarkers, one or more (e.g., at least 5, 10, 15, or 20 or more (e.g., all)) MIME biomarkers, one or more (e.g., at least 5, 10, 15, or 20 or more (e.g., all)) ESHAP biomarkers, one or more (e.g., at least 5, 10, 15, or 20 or more (e.g., all)) CBV biomarkers, one or more (e.g., at least 5, 10, 15, or 20 or more (e.g., all)) EPOCH biomarkers, one or more (e.g., at least 5, 10, 15, or 20 or more (e.g., all)) FCM biomarkers, one or more (e.g., at least 5, 10, 15, or 20 or more (e.g., all)) CVAD-A biomarkers, one or more (e.g., at least 5, 10, 15, or 20 or more (e.g., all)) CVAD-B biomarkers, one or more (e.g., at least 5, 10, 15, or 20 or more (e.g., all)) MINE biomarkers, one or more (e.g., at least 5, 10, 15, or 20 or more (e.g., all)) MOPP biomarkers, one or more (e.g., at least 5, 10, 15, or 20 or more (e.g., all)) CHOEP biomarkers, one or more (e.g., at least 5, 10, 15, or 20 or more (e.g., all)) BeICHOP biomarkers, one or more (e.g., at least 5, 10, 15, or 20 or more (e.g., all)) doxorubicin biomarkers, one or more (e.g., at least 5, 10, 15, or 20 or more (e.g., all)) bendamustine biomarkers, one or more (e.g., at least 5, 10, 15, or 20 or more (e.g., all)) ABVD biomarkers, one or more (e.g., at least 5, 10, 15, or 20 or more (e.g., all)) CHOP biomarkers, one or more (e.g., at least 5, 10, 15, or 20 or more (e.g., all)) prednisolone biomarkers, one or more (e.g., at least 5 or more (e.g., all)) GemOx biomarkers, one or more (e.g., at least 5 or more (e.g., all)) CVP biomarkers, one or more (e.g., at least 5, 10, 15, or 20 or more (e.g., all)) MTX biomarkers, one or more (e.g., at least 5, 10, 15, or 20 or more (e.g., all)) cyclophosphamide biomarkers, one or more (e.g., at least 5, 10, 15, or 20 or more (e.g., all)) vincristine biomarkers, one or more (e.g., at least 5, 10, 15, or 20 or more (e.g., all)) CEPP biomarkers, one or more (e.g., at least 5, 10, 15, or 20 or more (e.g., all)) COPP biomarkers, and/or one or more (e.g., at least 5, 10, 15, or 20 or more (e.g., all)) romidepsin biomarkers, as listed in Tables 1-3.

A microarray probe may be single-stranded or double-stranded. The probe may be labeled (e.g., detectably labeled with a fluorescent molecule, dye molecule, small molecule, epitope tag, barcode sequence, polypeptide, or any other detectable molecule). Probes can be detectably labeled and immobilized on a solid support to form the microarray. For example, probes can be either prefabricated and spotted to the surface or directly synthesized on to the surface (in situ) of the microarray. The microarray can also be configured such that the sequence and position of each member (e.g., probe) of the array is known. For example, a selection of biomarkers whose expression correlates with the presence of a disease (e.g., lymphoma (e.g., DLBCL)), an increased likelihood of developing the disease, or increased severity of the disease can be arrayed on a solid support. Hybridization of a labeled probe with a particular target nucleic acid (e.g., an mRNA corresponding to a biomarker of responsiveness) indicates that the sample from which the mRNA was derived expresses that biomarker (e.g., the biomarker of responsiveness to the treatment of interest). The microarray can be used to determine the level of biomarker expression, and thus can be used to assess the cancer patient's responsiveness to the drug or drug combination that is associated with the indicated biomarkers (see Table 1).

### Sequencing

Expression levels of biomarkers may be determined using sequencing technologies, such as next generation sequencing platforms (e.g., RNA-Seq), as described in Mortazavi et al., Nat. Methods 5: 621-628, 2008, hereby incorporated by reference. Sequencing methods for expression profiling offer a broader, dynamic range than microarrays and offer the possibility of quantifying sequences for which pre-designed probes are not available (e.g., novel transcripts, novel splice variants, or low-abundance transcripts). RNA-Seq is a robust technology for monitoring expression by direct sequencing the RNA molecules in a sample. This methodology may include fragmentation of RNA to an average length of, e.g., 200 nucleotides, conversion to cDNA by random priming, and synthesis of double-stranded cDNA (e.g., using the Just cDNA DoubleStranded cDNA Synthesis Kit from Agilent Technology). The cDNA may then be converted into a molecular library for sequencing by addition of sequence adapters for each library (e.g., from Illumina®/Solexa), and the resulting 50-100 nucleotide reads are mapped onto the genome.

Exemplary sequencing platforms suitable for use according to the invention include, but are not limited to, 454 pyrosequencing, Illumina sequencing by synthesis, SOLiD sequencing, Ion Torrent sequencing, and PacBio RS sequencing.

### Devices

Devices of the invention can be useful, e.g., for detecting the level of expression of at least one biomarker of responsiveness (e.g., those shown in Table 1) to a treatment or combination treatment of interest (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP). In some embodiments, the device includes at least one single-stranded nucleic acid (e.g., a probe) having at least 85% sequence identity (e.g., 85%, 90%, 95%, 97%, 98%, 99%, or 100% sequence identity) to a nucleic acid sequence that is complementary or identical to at least 5 (e.g., at least 10 or more) consecutive nucleotides of one or more biomarkers, in which the at least one single-stranded nucleic acid is sufficient for the detection of the level of expression of the one or more biomarkers. The device may be used to detect the expression level of a given biomarker by specific hybridization between the single-stranded nucleic acid and the biomarker (e.g., an mRNA, genomic DNA, or non-coding RNA), a nucleic acid encoding the biomarker (e.g., an mRNA), or a complementary nucleic acid thereof. The device may be or include a microarray. The device may also include or be used with reagents and materials for next generation sequence (e.g., sequencing by synthesis). The device may also include or be used with NanoString reagents and at least one nCounter cartridge. The device may be or include a protein array, which contains one or more protein binding moieties (e.g., proteins, antibodies, nucleic acids, aptamers, affibodies, lipids, phospholipids, small molecules, labeled variants of any of the above, and any other moieties useful for protein detection as well known in the art) capable of detectably binding to the polypeptide product(s) of one or more biomarkers (e.g., those shown in Tables 1-3) corresponding to the treatment or combination treatment of interest (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP).

### Kits

Kits of the invention can be used for determining the responsiveness of a patient to a treatment or combination treatment for cancer, e.g., lymphoma such as DLBCL (e.g., CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP, or an analog thereof) and/or for treating the cancer patient (e.g., the patient for lymphoma (e.g., DLBCL)) once responsiveness to a particular monotherapy or combination therapy (e.g., one or more of those shown in Table 1) has been determined. Kits of the invention can include reagents and/or materials for, e.g., collecting and/or purifying nucleic acids from biological samples (such as those obtained from a patient to be treated with a target drug(s) of the invention), reagents for amplifying such nucleic acids to produce an amplified sample, and/or at least one device of the invention as described herein. Reagents for amplifying nucleic acids may include, e.g., PCR reagents, including but not limited to DNA polymerase, RNA polymerase, PCR buffer, magnesium chloride solutions, nucleic acid primers (e.g., primers designed to target particular biomarkers of responsiveness to a target drug(s) of interest), and/or any other PCR reagents as are well known in the art. In some embodiments, a kit of the invention includes one or more probes capable of detecting one or more biomarkers of responsiveness to a target drug of interest. Such probes can, for example, include nucleic acids capable of hybridizing to the biomarker based on nucleic acid sequence complementarity. In certain embodiments, a probe has at least 85% sequence identity (e.g., 85%, 90%, 95%, 97%, 98%, 99%, or 100% sequence identity) to a nucleic acid sequence that is complementary or identical to at least 5 (e.g., at least 15) consecutive nucleotides of one or more biomarkers. The probes can be attached a solid surface, such as a microarray. The kit may include NanoString capture probes, NanoString reporter probes, and/or one or more nCounter cartridges. The kit may include reagents for next generation sequencing, including but not limited to poly(T) oligonucleotides, dye terminators, sequencing adapters, adapter ligation reagents, reverse transcriptase, primers (e.g., random primers), DNA-cleaving enzymes, polymerases, and/or any combination thereof. The kit may also be one that includes a protein array and/or reagents for detection of the polypeptide product(s) of one or more biomarkers of responsiveness, such as those described in Table 1.

The kits may also include compositions for treating cancer (e.g., lymphoma such as DLBCL). In particular, the kit may include one or more treatments or combinations treatments, including but not limited to CVP, COPE, MBVP, MTX, DHAP, prednisolone, cyclophosphamide, doxorubicin, vincristine, ICE, VIM, MIME, bendamustine, ESHAP, ABVD, BEACOPP, CBV, CEPP, COPP, EPOCH, FCM, GemOx, CVAD-A, CVAD-B, MINE, MOPP, Romidepsin, CHOP, CHOEP, and/or BeICHOP, as well as analogs thereof, such as prodrugs, derivatives, metabolites, enantiomers, and combinations thereof. Such kits may contain a composition or formulation of the treatment or combination treatment in question, or an enantiomer, prodrug, metabolite, or pharmaceutically acceptable salt thereof, together with the customary items for administering the therapeutic ingredient as are known in the art. In certain embodiments, a kit for administering a target drug may further include any of the reagents, materials, kits, or devices described above.

The following examples are intended to illustrate, rather than limit, the invention.

### EXAMPLES

### Example 1

### Predicting Response to First Line Treatment in Lymphoma

MicroRNA profiling was available from 116 patients with the diagnosis of diffuse large B-cell lymphoma (DLBCL), deposited under accession number GSE40239 in the National Center for Biological Information (NBCI, Bethesda, Md.). It can be accessed at ncbi.nlm.nih.gov. Details about the cohort and the production of data are available under accession number GSE40239.

For predicting response to first line treatment (using either the drug combination CHOP or the drug combination CHOEP), the corresponding microRNA profiles for CHOP and CHOEP for these treatments shown in Table 1 was determined and a predicted sensitivity calculated for each patient as

Prediction score=mean(microRNAs)

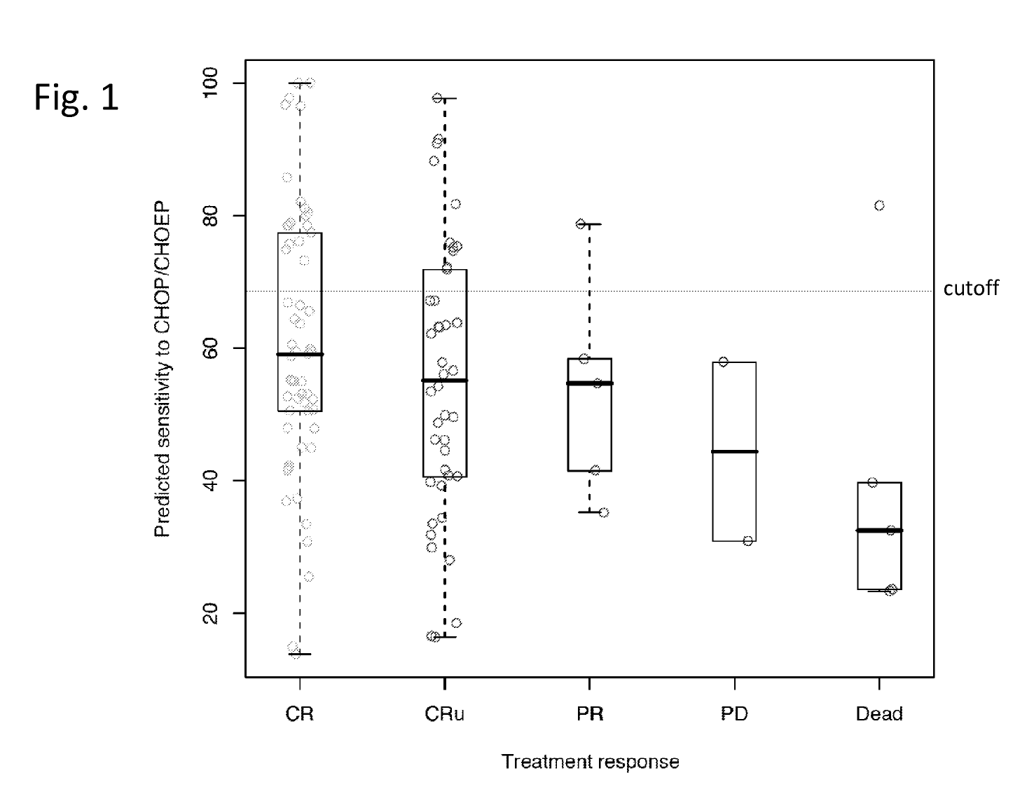

Each microRNA in the profile is given equal weight. Next, the prediction score was normalized to a scale from 0 to 100 by a linear transformation of the prediction score of all patient samples. FIG. 1 shows how the prediction score corresponds to the outcome of first line treatment. Patients that have a higher predicted sensitivity have a higher probability of a reduction in their tumor (complete or partial response).

An even better prediction can be obtained if the existing clinical International Prognostic Index (IPI score, standardly used in clinical evaluation of lymphoma patients) is combined with the prediction score as follows:

Combination score=Prediction score−20*IPI score

### Example 2

### Predicting Response to Second and Third Line Treatment in Lymphoma

Example 1 shows prediction of response to first line treatment in lymphoma. However, first line treatment is very effective in some lymphomas, like DLBCL, where around 80% of patients respond to first line treatment. Thus, doctors are unlikely to change first line treatment in response to a prediction by a statistical algorithm. When patients fail to respond or experience a relapse after first line treatment, the clinical situation is different for determining if the patient should be given the same treatment as in first line or receive a different combination treatment. There are many other available treatment combinations for lymphoma from which the physician may select. The response rate in second and third line treatments is much lower than in first line treatment. The response predictors described herein thus offer invaluable guidance.

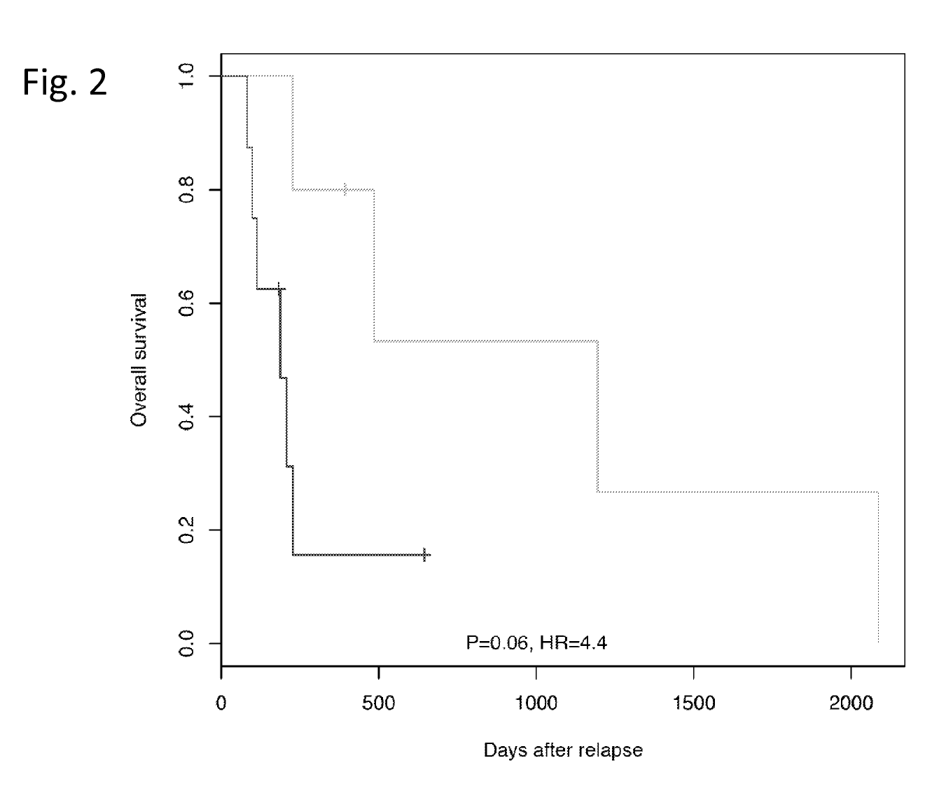

FIG. 2 shows 13 relapse patients from the cohort used in Example 1. The patients received one or more of the treatment combinations of COPE, DHAP, CVP, HDMTX, or MVBCNS. Regardless of treatment received, the corresponding microRNA profile for these treatments shown in Table 1 was determined and the sensitivity of the patient to that treatment was predicted. In FIG. 2, patients predicted sensitive to the treatment received are shown as the top curve and patients predicted resistant are shown in the bottom curve. The median survival for patients predicted sensitive was 3 years, whereas the median survival of the patients predicted resistant was 6 months. Among the eight patients that were predicted to not be sensitive to the received treatment, it was predicted that three would have been sensitive to another therapy, ICE (microRNA profile shown in Table 1) and one would have been sensitive to bendamustine (microRNA profile shown in Table 1).

### Example 3

### Application of Response Predictors to Clinical Practice

For a patient with the diagnosis of lymphoma, a biopsy may be analyzed using the same procedure and technology as described for Examples 1 and 2. MicroRNA may be extracted from FFPE using RecoverAll (Ambion, Inc 2130 Woodward St. Austin, Tex.). MicroRNA may be labeled using FlashTag HSR™ Biotin RNA Labeling Kit (Genisphere, PA) and analyzed using GeneChip® miRNA version 1.0 arrays (Affymetrix, CA).

For each treatment combination shown in Table 1, a prediction score may be calculated and compared to the predictions scores of the cohort used in Example 1. If the diagnosis is DLBCL, the cohort used in Example 1 may be used as a reference population. For those treatments where the prediction score is above the median of the Example 1 cohort, the clinical prediction is “sensitive” to the treatment of interest. For those treatments where the prediction score is below the median of the Example cohort 1 the clinical prediction is “resistant” to the treatment of interest.

The methods described may include the use of a tissue sample from a diagnostic biopsy, which may provide nucleic acid material for determining the level of one or more microRNA biomarkers (e.g., the set of biomarkers shown in Table 1) simultaneously, which allows for prediction of one or monotherapies or combination treatments that will likely be effective for the individual patient. For lymphoma diagnoses other than DLBCL, a reference population with the same diagnosis can be used as a control.

### Other Embodiments

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of predicting the responsiveness to treatment in a patient having diffuse large B-cell lymphoma comprising:
(a) determining a level of expression of one or more first biomarkers selected from the group consisting of hsa-miR-766_st and hsa-miR-34b_st in a biological sample from said patient, wherein said treatment is selected from the group consisting of:
i) cyclophosphamide, vincristine, and etoposide (COPE);
ii) methotrexate, carmustine, and teniposide (MBVP);
iii) dexamethasone, ara-c, and cisplatin (DHAP);
iv) ifosfamide, carboplatin, and etoposide (ICE);
v) etoposide, ifosfamide, and methotrexate (VIM);
vi) methyl-GAG, ifosfamide, methotrexate, and etoposide (MIME);
vii) etoposide, cytarabine, and cisplatin (ESHAP);
viii) cyclophosphamide, carmustine, and etoposide (CBV);
ix) cyclophosphamide, etoposide, vincristine, and doxorubicine (EPOCH);
x) fludarabine, cyclophosphamide, and mitoxantrone (FCM);
xi) cyclophosphamide, vincristine, doxorubicin, and dexamethasone (CVAD-A);
xii) methotrexate and cytarabine (CVAD-B);
xiii) ifosfamide, mitoxantrone, and etoposide (MINE);
xiv) mechlorethamine, vincristine, and procarbazine (MOPP);
xv) cyclophosphamide, vincristine, adriamycine, and etoposide (CHOEP);
xvi) cyclophosphamide, vincristine, adriamycine, and belinostat (BeICHOP);
xvii) methotrexate (MTX);
xviii) doxorubicin; and
xix) bendamustine; or
(b) determining the level of hsa-miR-766_st in a biological sample from said patient, wherein said treatment is selected from the group consisting of:
i) adriamycin, bleomycin, vinblastine, and dacarbazine (ABVD);
ii) cyclophosphamide, vincristine, adriamycine (CHOP); and
iii) prednisolone; or
(c) determining the level of hsa-miR-34b_st in a biological sample from said patient, wherein said treatment is gemcitabine and oxaliplatin (GemOx);
wherein said level of expression of hsa-miR-766_st and/or hsa-miR-34b_st indicates whether said patient is responsive to said treatment.

2.-20. (canceled)

21. The method of claim 1, wherein said method further comprises determining the level of expression of one or more second biomarkers in said biological sample from said patient or in a second biological sample from said patient, wherein the level of expression of said one or more second biomarkers indicates said patient is responsive to said treatment, wherein said treatment is selected from the group consisting of:
i) COPE and said one or more second biomarkers is selected from the group consisting of ACA48_x_st, ENSG00000202498_x_st, HBII-85-26_x_st, HBII-85-6_x_st, hsa-miR-106b-star_st, hsa-miR-1181_st, hsa-miR-124_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-631_st, hsa-miR-671-5p_st, and hsa-miR-93-star_st; and
ii) MBVP and said one or more second biomarkers is selected from the group consisting of ACA10_s_st, ACA11_st, ACA13_st, ACA18_x_st, ACA21_st, ACA40_x_st, ACA41_x_st, ACA48_st, ACA48_x_st, ACA51_x_st, ACA57_st, ACA61_st, ACA7_s_st, ACA9_st, ENSG00000199411_s_st, ENSG00000200879_st, ENSG00000200932_st, ENSG00000201859_x_st, ENSG00000202252_st, ENSG00000207002_st, ENSG00000207002_x_st, HBII-115_st, HBII-135_x_st, HBII-180A_x_st, HBII-180C_x_st, HBII-202_st, HBII-239_st, HBII-336_st, HBII-429_st, HBII-55_st, HBII-85-26_st, HBII-85-6_x_st, U104_st, U13_st, U17a_st, U17a_x_st, U17b_st, U17b_x_st, U22_st, U25_st, U27_st, U29_st, U3-2_s_st, U30_st, U31_x_st, U32A_x_st, U33_st, U34_st, U38A_st, U38B_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71a_st, U71b_x_st, U71d_st, U71d_x_st, U74_x_st, U78_s_st, U78_x_st, U83B_st, U89_st, U8_x_st, U95_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1299_st, hsa-miR-140-3p_st, hsa-miR-142-3p_st, hsa-miR-142-5p_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-491-3p_st, hsa-miR-595_st, hsa-miR-631_st, hsa-miR-663b_st, hsa-miR-671-5p_st, hsa-miR-768-3p_st, hsa-miR-768-5p_st, hsa-miR-769-5p_st, hsa-miR-874_st, hsa-miR-877-star_st, hsa-miR-92a-1-star_st, hsa-miR-92a_st, and hsa-miR-93-star_st; and
iii) DHAP and said one or more second biomarkers is selected from the group consisting of ACA7_s_st, ENSG00000199282_st, ENSG00000200879_st, ENSG00000201299_x_st, ENSG00000201859_x_st, ENSG00000202252_st, ENSG00000202498_x_st, HBII-202_st, HBII-336_st, HBII-429_st, HBII-438A_s_st, HBII-55_st, HBII-85-11_st, HBII-85-26_st, HBII-85-29_x_st, HBII-85-2_x_st, HBII-85-6_x_st, U104_st, U13_st, U17a_st, U17a_x_st, U17b_st, U17b_x_st, U3-2 s_st, U30_st, U33_st, U41_st, U48_st, U49A_st, U49A_x_st, U49B_s_st, U52_st, U55_st, U55_x_st, U57_st, U67_st, U71b_x_st, U78_s_st, U83_st, hsa-miR-106b-star_st, hsa-miR-1183_st, hsa-miR-1207-5p_st, hsa-miR-1268_st, hsa-miR-1281_st, hsa-miR-140-3p_st, hsa-miR-150_st, hsa-miR-155_st, hsa-miR-181a-star_st, hsa-miR-181a_st, hsa-miR-181b_st, hsa-miR-181c_st, hsa-miR-195-star_st, hsa-miR-198_st, hsa-miR-20b-star_st, hsa-miR-223_st, hsa-miR-297_st, hsa-miR-33b-star_st, hsa-miR-342-3p_st, hsa-miR-342-5p_st, hsa-miR-424-star_st, hsa-miR-432_st, hsa-miR-503_st, hsa-miR-574-5p_st, hsa-miR-613_st, hsa-miR-615-3p_st, hsa-miR-631_st, hsa-miR-768-5p_st, hsa-miR-877-star_st, hsa-miR-92a-2-star_st, and hsa-miR-938_st; and
iv) ICE and said one or more second biomarkers is selected from the group consisting of 14qII-14_st, 14qII-1_st, 14qII-26_st, ACA48_x_st, ENSG00000199282_st, ENSG00000201859_x_st, ENSG00000202498_x_st, HBII-85-26_st, HBII-85-6_x_st, U52_st, U55_st, U55_x_st, U8_x_st, hsa-miR-106b-star_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-181d_st, hsa-miR-195-star_st, hsa-miR-199b-3p_st, hsa-miR-297_st, hsa-miR-299-3p_st, hsa-miR-342-3p_st, hsa-miR-342-5p_st, hsa-miR-377-star_st, hsa-miR-409-3p_st, hsa-miR-409-5p_st, hsa-miR-410_st, hsa-miR-432_st, hsa-miR-487b_st, hsa-miR-615-3p_st, hsa-miR-631_st, hsa-miR-654-3p_st, hsa-miR-671-5p_st, hsa-miR-92b_st, hsa-miR-93-star_st, and hsa-miR-938_st; and
v) VIM and said one or more second biomarkers is selected from the group consisting of ACA10_s_st, ACA11_st, ACA13_st, ACA18_x_st, ACA21_st, ACA48_st, ACA48_x_st, ACA7_s_st, ENSG00000199411_s_st, ENSG00000200879_st, ENSG00000200932_st, ENSG00000201859_x_st, ENSG00000202252_st, ENSG00000202498_x_st, ENSG00000207002_st, HBII-115_st, HBII-180A_x_st, HBII-202_st, HBII-336_st, HBII-429_st, HBII-55_st, HBII-85-26_st, HBII-85-6_x_st, U104_st, U13_st, U17a_st, U17b_st, U17b_x_st, U25_st, U27_st, U29_st, U3-2_s_st, U30_st, U31_x_st, U32A_x_st, U33_st, U34_st, U38A_st, U41_st, U48_st, U49A_st, U49A_x_st, U49B_s_st, U52_st, U55_st, U55_x_st, U56_st, U56_x_st, U57_st, U64_st, U67_st, U67_x_st, U68_st, U68_x_st, U71b_x_st, U71d_x_st, U74_x_st, U83B_st, U8_x_st, hsa-miR-106a_st, hsa-miR-106b-star_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-18a-star_st, hsa-miR-18a_st, hsa-miR-195-star_st, hsa-miR-19a_st, hsa-miR-19b_st, hsa-miR-223_st, hsa-miR-25-star_st, hsa-miR-297_st, hsa-miR-33b-star_st, hsa-miR-423-3p_st, hsa-miR-423-5p_st, hsa-miR-663b_st, hsa-miR-768-3p_st, hsa-miR-768-5p_st, hsa-miR-769-5p_st, hsa-miR-92a-1-star_st, hsa-miR-92a_st, and hsa-miR-93-star_st; and
vi) MIME and said one or more second biomarkers is selected from the group consisting of ACA10_s_st, ACA11_st, ACA13_st, ACA18_x_st, ACA21_st, ACA40_x_st, ACA48_x_st, ACA51_x_st, ACA55_st, ACA61_st, ACA7_s_st, ACA9_st, ENSG00000199282_st, ENSG00000199411_s_st, ENSG00000200879_st, ENSG00000202252_st, ENSG00000202498_x_st, ENSG00000207002_st, ENSG00000207002_x_st, HBII-180A_x_st, HBII-180C_x_st, HBII-202_st, HBII-239_st, HBII-336_st, HBII-429_st, HBII-55_st, HBII-85-26_st, HBII-85-6_x_st, HBII-85-8_x_st, U104_st, U13_st, U17a_st, U17a_x_st, U17b_st, U17b_x_st, U22_st, U25_st, U26_st, U27_st, U28_st, U29_st, U3-2_s_st, U30_st, U31_x_st, U33_st, U34_st, U36A_st, U38A_st, U41_st, U43_x_st, U48_st, U49A_st, U49A_x_st, U49B_s_st, U52_st, U55_st, U55_x_st, U56_st, U56_x_st, U57_st, U59B_st, U64_st, U67_st, U68_st, U71a_st, U71b_x_st, U71d_x_st, U74_x_st, U75_st, U78_s_st, U78_x_st, U83B_st, U83_st, hsa-miR-106a_st, hsa-miR-106b-star_st, hsa-miR-1183_st, hsa-miR-1246_st, hsa-miR-124_st, hsa-miR-1254_st, hsa-miR-1275_st, hsa-miR-1281_st, hsa-miR-1307_st, hsa-miR-153_st, hsa-miR-17-star_st, hsa-miR-17_st, hsa-miR-181a-star_st, hsa-miR-181b_st, hsa-miR-18a-star_st, hsa-miR-18a_st, hsa-miR-18b_st, hsa-miR-195-star_st, hsa-miR-19a_st, hsa-miR-19b_st, hsa-miR-20a_st, hsa-miR-223_st, hsa-miR-25-star_st, hsa-miR-297_st, hsa-miR-33b-star_st, hsa-miR-423-3p_st, hsa-miR-423-5p_st, hsa-miR-425-star_st, hsa-miR-595_st, hsa-miR-615-3p_st, hsa-miR-663b_st, hsa-miR-768-3p_st, hsa-miR-768-5p_st, hsa-miR-769-5p_st, hsa-miR-92a-1-star_st, hsa-miR-92a_st, and hsa-miR-93-star_st; and
vii) ESHAP and said one or more second biomarkers is selected from the group consisting of ACA48_x_st, ACA7_s_st, ENSG00000201859_x_st, HBII-202_st, HBII-429_st, HBII-438A_s_st, HBII-55_st, HBII-85-11_st, HBII-85-26_st, HBII-85-2_x_st, HBII-85-6_x_st, HBII-85-8_x_st, U104_st, U17b_st, U17b_x_st, U33_st, U48_st, U52_st, U55_st, U55_x_st, U57_st, U67_st, U74_x_st, U78_s_st, U78_x_st, U95_st, hsa-miR-124_st, hsa-miR-1281_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-342-3p_st, hsa-miR-342-5p_st, hsa-miR-424-star_st, and hsa-miR-768-5p_st; and
viii) CBV and said one or more second biomarkers is selected from the group consisting of ACA48_x_st, ACA7_s_st, ENSG00000199282_st, ENSG00000202252_st, ENSG00000202498_x_st, ENSG00000207002_st, HBII-202_st, HBII-85-26_st, HBII-85-6_x_st, U104_st, U17a_st, U34_st, U41_st, U52_st, U55_st, U55_x_st, U57_st, U67_st, U74_x_st, hsa-miR-106a-star_st, hsa-miR-106b-star_st, hsa-miR-1181_st, hsa-miR-1183_st, hsa-miR-1228_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-223_st, hsa-miR-297_st, hsa-miR-339-5p_st, hsa-miR-342-3p_st, hsa-miR-342-5p_st, hsa-miR-432_st, hsa-miR-551b-star_st, hsa-miR-610_st, hsa-miR-615-3p_st, hsa-miR-631_st, hsa-miR-671-5p_st, hsa-miR-768-3p_st, hsa-miR-768-5p_st, hsa-miR-769-5p_st, hsa-miR-92b_st, hsa-miR-93-star_st, and hsa-miR-938_st; and
ix) EPOCH and said one or more second biomarkers is selected from the group consisting of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551 b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and hsa-miR-93_st; and
x) FCM and said one or more second biomarkers is selected from the group consisting of ACA7_s_st, HBII-438A_s_st, HBII-85-11_st, HBII-85-2_x_st, HBII-85-6_x_st, U104_st, U52_st, hsa-miR-1281_st, hsa-miR-1299_st, hsa-miR-140-3p_st, hsa-miR-155_st, hsa-miR-181a-2-star_st, hsa-miR-181a-star_st, hsa-miR-181a_st, hsa-miR-181b_st, hsa-miR-181c_st, hsa-miR-181d_st, hsa-miR-223_st, hsa-miR-342-3p_st, hsa-miR-342-5p_st, hsa-miR-34c-3p_st, hsa-miR-424-star_st, hsa-miR-489_st, hsa-miR-503_st, hsa-miR-768-5p_st, hsa-miR-874_st, and hsa-miR-92b_st; and
xi) CVAD-A and said one or more second biomarkers is selected from any one or more of ENSG00000201125_x_st, ENSG00000201859_x_st, ENSG00000202498_x_st, HBII-85-26_st, HBII-85-6_x_st, U17b_st, U17b_x_st, U48_st, U49A_st, U49A_x_st, U49B_s_st, U55_st, U55_x_st, hsa-miR-106a-star_st, hsa-miR-106b-star_st, hsa-miR-1183_st, hsa-miR-1207-5p_st, hsa-miR-1271_st, hsa-miR-1281_st, hsa-miR-128_st, hsa-miR-150_st, hsa-miR-181c-star_st, hsa-miR-195-star_st, hsa-miR-20b-star_st, hsa-miR-223_st, hsa-miR-297_st, hsa-miR-324-3p_st, hsa-miR-33b-star_st, hsa-miR-342-3p_st, hsa-miR-342-5p_st, hsa-miR-361-5p_st, hsa-miR-424-star_st, hsa-miR-424_st, hsa-miR-432_st, hsa-miR-503_st, hsa-miR-631_st, hsa-miR-671-5p_st, hsa-miR-769-5p_st, and hsa-miR-92a-2-star_st; and
xii) CVAD-B and said one or more second biomarkers is selected from the group consisting of ACA21_st, ACA48_st, ACA48_x_st, ENSG00000200879_st, HBII-202_st, HBII-336_st, HBII-429_st, HBII-438A_s_st, HBII-55_st, U104_st, U17a_x_st, U17b_st, U17b_x_st, U25_st, U27_st, U29_st, U30_st, U31_x_st, U32A_x_st, U33_st, U34_st, U48_st, U49A_st, U49A_x_st, U49B_s_st, U55_st, U55_x_st, U56_st, U56_x_st, U57_st, U67_st, U74_x_st, U78_s_st, U78_x_st, hsa-miR-153_st, hsa-miR-17_st, hsa-miR-181a-star_st, hsa-miR-18a-star_st, hsa-miR-18a_st, hsa-miR-223_st, hsa-miR-25-star_st, hsa-miR-297_st, hsa-miR-33b-star_st, hsa-miR-663b_st, hsa-miR-768-5p_st, and hsa-miR-92a_st; and
xiii) MINE and said one or more second biomarkers is selected from the group consisting of 14qII-14_st, 14qII-14_x_st, 14qII-1_st, 14qII-26_st, 14qII-26_x_st, ENSG00000202498_x_st, HBII-85-26_st, HBII-85-6_x_st, U104_st, U52_st, U55_st, U55_x_st, hsa-miR-124_st, hsa-miR-127-3p_st, hsa-miR-1281_st, hsa-miR-1299_st, hsa-miR-140-3p_st, hsa-miR-181a-star_st, hsa-miR-181a_st, hsa-miR-181b_st, hsa-miR-181c-star_st, hsa-miR-181c_st, hsa-miR-181d_st, hsa-miR-195-star_st, hsa-miR-297_st, hsa-miR-299-3p_st, hsa-miR-29b-2-star_st, hsa-miR-339-5p_st, hsa-miR-33b-star_st, hsa-miR-342-3p_st, hsa-miR-342-5p_st, hsa-miR-377-star_st, hsa-miR-379_st, hsa-miR-381_st, hsa-miR-409-3p_st, hsa-miR-409-5p_st, hsa-miR-411_st, hsa-miR-432_st, hsa-miR-433_st, hsa-miR-487b_st, hsa-miR-493_st, hsa-miR-654-3p_st, hsa-miR-671-5p_st, hsa-miR-768-3p_st, hsa-miR-768-5p_st, hsa-miR-92b_st, and hsa-miR-938_st; and
xiv) MOPP and said one or more second biomarkers is selected from the group consisting of ACA13_st, ACA21_st, ACA48_x_st, ENSG00000199282_st, ENSG00000201299_x_st, HBII-239_st, HBII-336_st, HBII-85-26_st, HBII-85-2_x_st, HBII-85-6_x_st, U104_st, U13_st, U22_st, U55_st, U55_x_st, U74_x_st, U8_x_st, hsa-miR-124_st, hsa-miR-1281_st, hsa-miR-1299_st, hsa-miR-140-3p_st, hsa-miR-181a-star_st, hsa-miR-181a_st, hsa-miR-181b_st, hsa-miR-181c_st, hsa-miR-195-star_st, hsa-miR-297_st, hsa-miR-29b-2-star_st, hsa-miR-331-5p_st, hsa-miR-33b-star_st, hsa-miR-342-3p_st, hsa-miR-342-5p_st, hsa-miR-424-star_st, hsa-miR-432_st, hsa-miR-489_st, hsa-miR-768-3p_st, hsa-miR-768-5p_st, hsa-miR-874_st, hsa-miR-877-star_st, and hsa-miR-92b_st; and
xv) CHOEP and said one or more second biomarkers is selected from the group consisting of ACA48_x_st, ENSG00000202498_x_st, HBII-85-26_st, HBII-85-6_x_st, U55_st, U55_x_st, hsa-miR-106b-star_st, hsa-miR-106b_st, hsa-miR-1181_st, hsa-miR-124_st, hsa-miR-127-3p_st, hsa-miR-1281_st, hsa-miR-1299_st, hsa-miR-140-3p_st, hsa-miR-195-star_st, hsa-miR-25-star_st, hsa-miR-33b-star_st, hsa-miR-409-3p_st, hsa-miR-432_st, hsa-miR-551b-star_st, hsa-miR-629-star_st, hsa-miR-652_st, hsa-miR-654-3p_st, hsa-miR-671-5p_st, hsa-miR-877-star_st, hsa-miR-92b_st, hsa-miR-93-star_st, and hsa-miR-93_st and
xvi) BeICHOP and said one or more second biomarkers is selected from the group consisting of ACA10_s_st, ACA13_st, ACA18_x_st, ACA48_x_st, ACA9_st, ENSG00000200932_st, HBII-180A_x_st, HBII-55_st, HBII-85-26_st, HBII-85-6_x_st, U104_st, U13_st, U17a_st, U17b_st, U17b_x_st, U22_st, U38A_st, U49B_s_st, U51_st, U55_st, U55_x_st, U56_st, U56_x_st, U57_st, U67_st, U71a_st, hsa-miR-106a_st, hsa-miR-106b-star_st, hsa-miR-106b_st, hsa-miR-1181_st, hsa-miR-1228_st, hsa-miR-1246_st, hsa-miR-124_st, hsa-miR-1254_st, hsa-miR-1268_st, hsa-miR-1281_st, hsa-miR-1299_st, hsa-miR-17-star_st, hsa-miR-181c-star_st, hsa-miR-18a-star_st, hsa-miR-18a_st, hsa-miR-18b_st, hsa-miR-195-star_st, hsa-miR-19a_st, hsa-miR-19b_st, hsa-miR-25-star_st, hsa-miR-25_st, hsa-miR-297_st, hsa-miR-33b-star_st, hsa-miR-346_st, hsa-miR-551b-star_st, hsa-miR-593-star_st, hsa-miR-595_st, hsa-miR-611_st, hsa-miR-629-star_st, hsa-miR-629_st, hsa-miR-631_st, hsa-miR-652_st, hsa-miR-671-5p_st, hsa-miR-769-5p_st, hsa-miR-877-star_st, hsa-miR-93-star_st, and hsa-miR-93_st; and
xvii) MTX and said one or more second biomarkers is selected from the group consisting of ACA10_s_st, ACA18_x_st, ACA48_x_st, ACA51_x_st, ENSG00000199411_s_st, ENSG00000200879_st, HBII-180A_x_st, HBII-180C_x_st, HBII-202_st, HBII-429_st, HBII-55_st, U104_st, U17a_st, U17b_st, U17b_x_st, U25_st, U26_st, U27_st, U29_st, U3-2_s_st, U30_st, U31_x_st, U33_st, U38A_st, U48_st, U49A_st, U49A_x_st, U49B_s_st, U49B_x_st, U55_st, U55_x_st, U56_st, U56_x_st, U57_st, U67_st, U67_x_st, U74_x_st, U78_x_st, U89_st, hsa-miR-106a_st, hsa-miR-1246_st, hsa-miR-1254_st, hsa-miR-1275_st, hsa-miR-17-star_st, hsa-miR-17_st, hsa-miR-18a-star_st, hsa-miR-18a_st, hsa-miR-18b_st, hsa-miR-19a_st, hsa-miR-19b_st, hsa-miR-20a_st, hsa-miR-25-star_st, hsa-miR-297_st, hsa-miR-33b-star_st, hsa-miR-663b_st, hsa-miR-768-5p_st, hsa-miR-92a-1-star_st, hsa-miR-92a_st, and hsa-miR-936_st; and
xviii) doxorubicin and said one or more second biomarkers is selected from the group consisting of ACA13_st, ACA48_x_st, U104_st, U55_st, U55_x_st, U74_x_st, hsa-miR-106a-star_st, hsa-miR-106b-star_st, hsa-miR-106b_st, hsa-miR-124_st, hsa-miR-1281_st, hsa-miR-1299_st, hsa-miR-140-3p_st, hsa-miR-195-star_st, hsa-miR-297_st, hsa-miR-29b-2-star_st, hsa-miR-33b-star_st, hsa-miR-342-3p_st, hsa-miR-342-5p_st, hsa-miR-432_st, hsa-miR-550-star_st, hsa-miR-629-star_st, hsa-miR-629_st, hsa-miR-652_st, hsa-miR-654-3p_st, hsa-miR-671-5p_st, hsa-miR-768-3p_st, hsa-miR-877-star_st, hsa-miR-93-star_st, and hsa-miR-93_st; and
xix) bendamustine and said one or more second biomarkers is selected from the group consisting of ACA11_st, ACA24_x_st, ACA7_s_st, HBII-115_st, HBII-438A_s_st, HBII-85-11_st, U13_st, U49B_s_st, hsa-miR-106b-star_st, hsa-miR-128_st, hsa-miR-1299_st, hsa-miR-142-5p_st, hsa-miR-153_st, hsa-miR-155_st, hsa-miR-15a-star_st, hsa-miR-15a_st, hsa-miR-181a-star_st, hsa-miR-181a_st, hsa-miR-181b_st, hsa-miR-181c_st, hsa-miR-20b-star_st, hsa-miR-29b-2-star_st, hsa-miR-29c-star_st, hsa-miR-29c_st, hsa-miR-342-5p_st, hsa-miR-361-5p_st, hsa-miR-363-star_st, hsa-miR-647_st, and hsa-miR-93-star_st.

22.-52. (canceled)

53. The method of claim 1, further comprising administering said treatment to said patient, wherein said determining occurs: i) prior to said administration; ii) substantially concurrent with said administration; or iii) after said administration.

54.-56. (canceled)

57. The method of claim 53, wherein said determining step occurs multiple times.

58.-61. (canceled)

62. The method of claim 1, further comprising, prior to said determining, providing said biological sample obtained from said patient, wherein said biological sample is selected from formalin-fixed paraffin embedded (FFPE) tissue or fresh frozen tissue.

63.-64. (canceled)

65. The method of claim 62, wherein said biological sample is obtained from a tumor, and wherein said biological sample comprises a tumor biopsy.

66. (canceled)

67. The method of claim 65, wherein said biological sample comprises a tumor biopsy from a patient having suffered a relapse of diffuse large B-cell lymphoma after a first, second, or third line therapy.

68.-124. (canceled)

125. The method of claim 21, wherein the level of expression of at least one, at least two, at least three, at least four, at least five, or each of said second biomarkers is determined in said patient.

126.-130. (canceled)

131. The method of claim 21, further comprising administering one or more additional therapies to said patient, wherein said one or more additional therapies is administered: i) concurrently with administration of said treatment, ii) prior to administration of said treatment, or iii) after administration of said treatment.

132.-135. (canceled)

136. The method of claim 131, wherein said one or more additional therapies comprises one or more additional therapeutic agents, surgery, or radiation therapy.

137. The method of claim 136, wherein said one or more additional therapeutic agents is selected from the group consisting of cyclophosphamide, vincristine, and etoposide (COPE); methotrexate, carmustine, and teniposide (MBVP); dexamethasone, ara-c, and cisplatin (DHAP); ifosfamide, carboplatin, and etoposide (ICE); etoposide, ifosfamide, and methotrexate (VIM); methyl-GAG, ifosfamide, methotrexate, and etoposide (MIME); etoposide, cytarabine, methylprednisolone, and cisplatin (ESHAP); cyclophosphamide, carmustine, and etoposide (CBV); cyclophosphamide, etoposide, vincristine, and doxorubicine (EPOCH); fludarabine, cyclophosphamide, and mitoxantrone (FCM); cyclophosphamide, vincristine, doxorubicin, and dexamethasone (CVAD-A); methotrexate and cytarabine (CVAD-B); ifosfamide, mitoxantrone, and etoposide (MINE); mechlorethamine, vincristine, and procarbazine (MOPP); cyclophosphamide, vincristine, adriamycine, and etoposide (CHOEP); cyclophosphamide, vincristine, adriamycine, and belinostat (BeICHOP); methotrexate (MTX); doxorubicin; bendamustine; adriamycin, bleomycin, vinblastine, and dacarbazine (ABVD); cyclophosphamide, vincristine, adriamycine (CHOP); prednisolone; and gemcitabine and oxaliplatin (GemOx).

138. The method of claim 21, wherein said treatment is administered to said patient intravenously, orally, intraperitoneally, intramuscularly, topically, rectally, cutaneously, subcutaneously, nasally, intracerebroventricularly, intraparenchymally, intrathecally, intracranially, ocularly, via inhalation, or through the skin.

139. (canceled)

140. The method of claim 21, further comprising administering said treatment two or more times.

141.-148. (canceled)

149. The method of claim 21, wherein said treatment is administered in dosage form.

150. The method of claim 1, wherein said patient is a human.

151.-152. (canceled)

153. The method of claim 1, wherein said patient has suffered a relapse of diffuse large B-cell lymphoma.

154. The method of claim 1, wherein said level of expression of said first biomarkers is determined by detecting the level of microRNA in the biological sample.

155. The method of claim 1, wherein said method further comprises, prior to said determining, amplifying hsa-miR-766_st and/or hsa-miR-34b_st nucleic acid molecules in said biological sample or reverse transcribing hsa-miR-766_st and/or hsa-miR-34b in said biological sample, further wherein the product of said amplification or said reverse transcription is cDNA.

156.-158. (canceled)

159. The method of claim 1, wherein said level of expression of said first biomarkers is determined using sequencing.

160. (canceled)

161. The method of claim 21, wherein said level of expression of said first biomarkers or said second biomarkers is determined using a microarray, wherein said microarray comprises a plurality of nucleic acid probes, and wherein each of said nucleic acid probes has a length of about 5 nucleotides.

162. The method of claim 161, wherein each of said nucleic acid probes are configured to hybridize to a target RNA molecule or a target cDNA molecule.

163. (canceled)

164. The method of claim 162 or 163, wherein the nucleic acid sequence of said target RNA molecule or said target cDNA molecule is identical or complementary to all or a portion of the nucleic acid sequence of at least one of said first biomarkers or said second biomarkers.

165.-166. (canceled)

167. The method of claim 1, wherein said level of expression of hsa-miR-766_st is determined using a probe capable of hybridizing to a nucleic acid molecule having the sequence of SEQ ID NO. 246, wherein said probe comprises a nucleic acid sequence comprising at least 15 continuous nucleotides and at least 85% sequence identity to a sequence complementary to SEQ ID NO. 246.

168. (canceled)

169. The method of claim 1, wherein said level of expression of hsa-miR-34b_st is determined using a probe capable of hybridizing to a nucleic acid molecule having the sequence of SEQ ID NO. 185, wherein said probe comprises a nucleic acid sequence comprising at least 15 continuous nucleotides and at least 85% sequence identity to a sequence complementary to SEQ ID NO. 185.

170. (canceled)

171. The method of claim 21, further comprising determining the level of expression of one or more of said second biomarkers in a biological sample from said patient, wherein said level of expression of said second biomarkers is determined using a probe capable of hybridizing to a nucleic acid molecule comprising the nucleic acid sequence of said second biomarkers, wherein said second biomarkers are selected from one or more of the biomarkers of Table 1, and wherein said probe comprises a sequence comprising at least 15 continuous nucleotides and at least 85% sequence identity to a sequence complementary to any one of SEQ ID NOs. 1-263.

172.-174. (canceled)

175. The method of claim 1, further comprising converting said level of expression of one or more of said first biomarkers into a mean score for said treatment, wherein said mean score identifies the responsiveness of said patient to said treatment.

176. (canceled)

177. The method of claim 1, wherein the patient is predicted to be responsive to said treatment if the level of expression of said first biomarkers is statistically different from the level of expression of said first biomarkers in a control.

178. The method of claim 177, wherein:
(a) if the level of expression of said first biomarkers indicates that the patient is responsive to said treatment, said method further comprises administering said treatment to the patient as a first cancer treatment; or
(b) if the level of expression of said first biomarkers indicates that the patient is non-responsive to said treatment, said method further comprises administering a second cancer treatment that is different from the first cancer treatment.

179. The method of claim 21, wherein the patient is predicted to be responsive to said treatment if the level of expression of one or more of said second biomarkers is statistically different from the level of expression of one or more of said second biomarkers in a control.

180. The method of claim 179, wherein:
(a) if the level of expression of said second biomarker indicates that the patient is responsive to said treatment, said method further comprises administering said treatment to the patient as a first cancer treatment; or
(b) if the level of expression of second biomarker indicates that the patient is non-responsive to said treatment, said method further comprises administering a second cancer treatment that is different from the first cancer treatment.

181. A method of treating a patient having diffuse large B-cell lymphoma, said method comprising:
(a) administering a first cancer treatment to the patient, wherein the patient was previously determined to be responsive to the first cancer treatment according to the method of claim 1; or
(b) administering a second cancer treatment that is different from the first cancer treatment to the patient, wherein the patient was previously determined to be non-responsive to the first cancer treatment according to the method of claim 1, wherein the first cancer treatment is selected from the group consisting of:
i) cyclophosphamide, vincristine, and etoposide (COPE);
ii) methotrexate, carmustine, and teniposide (MBVP);
iii) dexamethasone, ara-c, and cisplatin (DHAP);
iv) ifosfamide, carboplatin, and etoposide (ICE);
v) etoposide, ifosfamide, and methotrexate (VIM);
vi) methyl-GAG, ifosfamide, methotrexate, and etoposide (MIME);
vii) etoposide, cytarabine, and cisplatin (ESHAP);
viii) cyclophosphamide, carmustine, and etoposide (CBV);
ix) cyclophosphamide, etoposide, vincristine, and doxorubicine (EPOCH);
x) fludarabine, cyclophosphamide, and mitoxantrone (FCM);
xi) cyclophosphamide, vincristine, doxorubicin, and dexamethasone (CVAD-A);
xii) methotrexate and cytarabine (CVAD-B);
xiii) ifosfamide, mitoxantrone, and etoposide (MINE);
xiv) mechlorethamine, vincristine, and procarbazine (MOPP);
xv) cyclophosphamide, vincristine, adriamycine, and etoposide (CHOEP);
xvi) cyclophosphamide, vincristine, adriamycine, and belinostat (BeICHOP);
xvii) methotrexate (MTX);
xviii) doxorubicin;
xix) bendamustine;
xx) adriamycin, bleomycin, vinblastine, and dacarbazine (ABVD);
xxi) cyclophosphamide, vincristine, adriamycine (CHOP); and
xxii) prednisolone; and
xxiii) gemcitabine and oxaliplatin (GemOx).

182. The method of claim 181, wherein the patient in (b) is administered both the first cancer treatment and the second cancer treatment.


SEQUENCE LISTING (263 sequences)

SEQ ID NO: 1 - DNA, 74 bp, Homo sapiens
tggaccaatgatgacaaataccggcgtatgagtcttggatgatgaataatacgtgtctggaactctgaggtcca

SEQ ID NO: 2 - DNA, 74 bp, Homo sapiens
tggaccaatgatgacaactgccggcgtatgagtgttgggtgatgaataatacgtgtctagaactctgaggtcca

SEQ ID NO: 3 - DNA, 74 bp, Homo sapiens; identical to SEQ ID NO: 2

SEQ ID NO: 4 - DNA, 71 bp, Homo sapiens
tggacctatgatgatgactggtggcgtatgagtcattgacggtgaatacaggtctggaagtctgaggtcca

SEQ ID NO: 5 - DNA, 71 bp, Homo sapiens; identical to SEQ ID NO: 4

SEQ ID NO: 6 - DNA, 71 bp, Homo sapiens
tggatcgatgatgactaccggtggcgtatgagtcatatgtgatgaatacgtgtttggaactctgaggtcca

SEQ ID NO: 7 - DNA, 71 bp, Homo sapiens
tggatcgatgatgagcactggtggagtatgagtcacatacgatgaatacgtgtctggaactctgaggtcca

SEQ ID NO: 8 - DNA, 71 bp, Homo sapiens; identical to SEQ ID NO: 7

SEQ ID NO: 9 - DNA, 74 bp, Homo sapiens
tggaccaatgatgaccactggtggcgtttgagtcatggacgatgaatactacgtgtctgaaactctgaggtcca

SEQ ID NO: 10 - DNA, 133 bp, Homo sapiens
ggtctctcagctccgcttaaccacacgggtccagtgtgtgcttggcgtgttttcagggaggcagagaaaggctctcctaatgcacgacagacccgcccagaatggcctctctgttcctaggagtgcgacaatt

SEQ ID NO: 11 - DNA, 125 bp, Homo sapiens
gcgctcactaaggctcggtcctctccacgtggtcctgacctgtcctctgtgagcaagagaaacaggactggtttgggggtgtcctgtctcagtggacacaggacaccacggttttcagtacaaca

SEQ ID NO: 12 - DNA, 133 bp, Homo sapiens
agcctttgtgttgcccattcactttggaaactagtgaatgtggtgtcaaaaaaggcgtaaattaaacgctttgcagccttttcctgcccttaaatttgatacctttggtgtaggagctgcataagtaacagtt

SEQ ID NO: 13 - DNA, 132 bp, Homo sapiens
gttgaggtctatcccgatggggcttttcctgtagcctgcacatcgttggaaacgcctcatagagtaactctgtggttttactttactcacaggactattgttagatctgtgggaaggaattacaagacagtt

SEQ ID NO: 14 - DNA, 133 bp, Homo sapiens
cccccttttaaaagcactcaatgggcctgtggctaatgacctattgagccgtcaagaaaggggagagtgaaaacatcgcttttgggtgaagtggcaacatgtgttgtttgcttcaatcggtggtgtgacaagg

SEQ ID NO: 15 - DNA, 131 bp, Homo sapiens
ctccatgtatctttgggacctgtcagccgtggcagtctcccttcctagccatggaagagcatatccttgtttattggcaaagctgtcaccatttaattggtatcagattctgacttgcacaagtaacattc

SEQ ID NO: 16 - DNA, 127 bp, Homo sapiens
tgcacttatgtatgtttttgtttaacgtggacaaagacttacagataggtgcaaaaaataaatcctcttttgcaacccagaactcattgttcagtatgagttttgatacatataagaagggatatta

SEQ ID NO: 17 - DNA, 132 bp, Homo sapiens
ttccacagctactggtctgcagctgttcttatggtagcagttgtggcattcctctgtgggaaagaaactgttaacacaaacacctctttcttagcaaaacagaaagtgggtatatatgtgtgacagacacaa

SEQ ID NO: 18 - DNA, 135 bp, Homo sapiens
tgtccctgacctgggtagagtggcatctggttggtgatgcccatctcatatcagccagggacaaagcaactccttgttcatcccagcttggcttttgatccgtgcccatgcctggttcatgccttggacacatag

SEQ ID NO: 19 - DNA, 135 bp, Homo sapiens; identical to SEQ ID NO: 18

SEQ ID NO: 20 - DNA, 132 bp, Homo sapiens
ggcctcctggtgcttaccacaggctgtgttcttacactgactgtatagaaagaggaggtagagtaaacctaccccatatacacctcagctcaggccctgtgcctggtctgtattgtgaatgggggaacatag

SEQ ID NO: 21 - DNA, 137 bp, Homo sapiens
gagcacctgaatctttcccattccttgctgcctcgtgccggtgtggggacagatggtgctacagaatgagcagaggaaatccagacaggttgttttccatttgtcttggggcctgtctctacagctctgccacattt

SEQ ID NO: 22 - DNA, 137 bp, Homo sapiens
ttgtcctggcctatttttctgctcccctgtgctcagttctaacagggtagtctggcaggacacacagcaattccctctcagtttaggagggccgtcctaagaatagggctggctcttaaaggcacgagaggacaatt

SEQ ID NO: 23 - DNA, 130 bp, Homo sapiens
atcctcctgatccctttcccatcggatctgaacactggtcttggtggtcgtaaaaggaggaaaagtaatagtgaagctggcctaaatgttgtaatctggtatatggcatgtgggctagtttcagacaggt

SEQ ID NO: 24 - DNA, 139 bp, Homo sapiens
gacctcctgggatcgcatctggagagtgcctagtattctgccagcttcggaaagggagggaaagcaagcctggcagaggcacccattccattcccagcttgctccgtagctggcgattggaagacactctgcgacagtg

SEQ ID NO: 25 - DNA, 133 bp, Homo sapiens
tagcaagcctccagcgtgcttgggtctgcggtgaccctatgcattccttcagtgcttgctagaacagttttgaaacggtttgaggccttgccctgctccatccagagcaaggttatagaaatttcagacaatg

SEQ ID NO: 26 - DNA, 131 bp, Homo sapiens
tggcaagcctccagcgtgcgtgggtctgtggtgatgtgtgcattcctacactgcttgccagaacagtattgaaatagtggagcgtttgtcctgctccatccagagcaaggttatagaaatttcagacaatt

SEQ ID NO: 27 - DNA, 86 bp, Homo sapiens
tcttagtgacataattctaatagtttgttccgaccttccactgtggactcaatagcagggagatgaagaggacagtgattgcatga

SEQ ID NO: 28 - DNA, 85 bp, Homo sapiens
tcacaatgatgaatggtccaaaacattcgcggtttccaccagaattcaaggtgttggcaactaccttccttggatgtctgagtga

SEQ ID NO: 29 - DNA, 79 bp, Homo sapiens
gccgtgtgaggccacctctgccatgctgcaggctccccagcacgcagtggggaacccaaccttggagagccgagccggg

SEQ ID NO: 30 - DNA, 127 bp, Homo sapiens
ctggggaattcaaacttgtgttaagaaaatgtgtcccagtgtgcaatggctgcaaacagcagcttccttggtagtgtatgcagcctgtttgttgtacgggttgctctaaagggccttggagacagtc

SEQ ID NO: 31 - DNA, 68 bp, Homo sapiens
actcactgatgagtagcttctgactttcattctgagtttgctgaacccagatgccattcctgggaagg

SEQ ID NO: 32 - DNA, 68 bp, Homo sapiens
cctcattgattagtagcttctgacttttgttctgagtttgctgaagctagatgccattccagataaga

SEQ ID NO: 33 - DNA, 88 bp, Homo sapiens
tcgctgtgatgagtgattgttaaacattcgtagtttccaccaaaagcttggctaatgatggcaacaccttccttggatgtctgagcga

SEQ ID NO: 34 - DNA, 92 bp, Homo sapiens
agatcattgatgacttccatatatccattccttggaaagctgaacaacatgagtgaaaactctactgaaaaaagaaaagaaatgggaggccg

SEQ ID NO: 35 - DNA, 131 bp, Homo sapiens
tgcagctgtgtcaaatttggtgcctgccctgcctgtgtaggcactgacccagaaggctgccacagaaacactgactcatgggccctgttcctgtgtctcgggctcagggataaatttggttacagatacca

SEQ ID NO: 36 - DNA, 131 bp, Homo sapiens; identical to SEQ ID NO: 35

SEQ ID NO: 37 - DNA, 110 bp, Homo sapiens
tgcccagtgatgacaccatccttgctccccgtgccccccaggggctatgggcgacaccatggctgcccctgggctgggccagtggggccaatgcccaggggctgagggca

SEQ ID NO: 38 - DNA, 73 bp, Homo sapiens
aaatgatgaaatcacccaaaatagctggaattaccggcagattgtgtagtggtgaacctatggttttctgaag

SEQ ID NO: 39 - DNA, 97 bp, Homo sapiens
ccggggcctccatgatgtccagcactgggctccgactgccactgaggacacggtgccccccgggacctttgacacccgggggtctgaggggccctgg

SEQ ID NO: 40 - DNA, 97 bp, Homo sapiens
ctggggctcccatgatgtccagcactgggctctgatcacccctgaggacacagtgcaccccaggacctttgacacctgggggtctgaggggccccag

SEQ ID NO: 41 - DNA, 72 bp, Homo sapiens
cgcgtgatgacattctccggaatcgctgtacggccttgatgaaagcacatttgaacccttttccatctgatt

SEQ ID NO: 42 - DNA, 86 bp, Homo sapiens
tgtgtgttggaggatgaaagtacggagtgatccatcggctaagtgtcttgtcacaatgctgacactcaaactgctgacagcacacg

SEQ ID NO: 43 - DNA, 74 bp, Homo sapiens
tggccaaggatgagaactctaatctgattttatgtgcttctgctgtgatggattaaaggatttacctgaggcca

SEQ ID NO: 44 - DNA, 76 bp, Homo sapiens
gctgtacatgatgacaactggctccctctactgaactgccatgaggaaactgccatgtcacccttctgattacagc

SEQ ID NO: 45 - DNA, 67 bp, Homo sapiens
ggatcgatgatgagaataattgtctgaggatgctgagggactcattccagatgtcaatctgaggtcc

SEQ ID NO: 46 - DNA, 75 bp, Homo sapiens
ttgcagtgatgacttgcgaatcaaatctgtcaatcccctgagtgcaatcactgatgtctccatgtctctgagcaa

SEQ ID NO: 47 - DNA, 94 bp, Homo sapiens
tggatcaatgatgacttccatacgtgggttccttggaaagttgaacaaaatgagtgaaaactttatactgtcatcctcttcaaactgaggtcca

SEQ ID NO: 48 - DNA, 98 bp, Homo sapiens
tggatcgatgatgactataaaaaaaatggatctcatcggaatctgaacaaaatgagtgaccaaatcatttctgtgccacttctgtgagctgaggtcca

SEQ ID NO: 49 - DNA, 85 bp, Homo sapiens
tggatcgatgatgacttaaaaaaatggaaaccttggaaatctgaacaaaatgagtgaccaagacacttctgtgagctgaggtcca

SEQ ID NO: 50 - DNA, 97 bp, Homo sapiens
tggatcgatgatgagtccccaaaaaaaacattccttggaaaagctgaacaaaatgagtgaaaactcataccgtcattctcatcggaactgaggtcca

SEQ ID NO: 51 - DNA, 98 bp, Homo sapiens
tggatcgatgatgagtcctccaaaaaaaacattccttggaaaagctgaacaaaatgagtgaaaactcataccgtcattctcatcggaactgaggtcca

SEQ ID NO: 52 - DNA, 97 bp, Homo sapiens
tggatcgatgatgagtcctccaaaaaaacattccttggaaaagctgaacaaaatgagtgagaactcataccgtcgttctcatcggaactgaggtcca

SEQ ID NO: 53 - DNA, 80 bp, Homo sapiens
ggcctgctgtgatgacattccaattaaagcacgtgttagactgctgacgcgggtgatgcgaactggagtctgagcctgcc

SEQ ID NO: 54 - DNA, 105 bp, Homo sapiens
gatccttttgtagttcatgagcgtgatgattgggtgttcatacgcttgtgtgagatgtgccacccttgaaccttgttacgacgtgggcacattacccgtctgacc

SEQ ID NO: 55 - DNA, 207 bp, Homo sapiens
tccaacgtggatacacccgggaggtcactctccccgggctctgtccaagtggcgtaggggagcatagggctctgccccatgatgtacaagtccctttccacaacgttggaaataaagctgggcctcgtgtctgcgcctgcatattcctacagcttcccagagtcctgtcgacaattactggggagacaaaccatgcaggaaacagcc

SEQ ID NO: 56 - DNA, 207 bp, Homo sapiens; identical to SEQ ID NO: 55

SEQ ID NO: 57 - DNA, 205 bp, Homo sapiens
tccaacgtggataccctgggaggtcactctccccaggctctgtccaagtggcataggggagcttagggctctgccccatgatgtacagtccctttccacaacgttgaagatgaagctgggcctcgtgtctgcgcctgcatattcctacagcttcccagagtcctgtggacaatgactggggagacaaaccatgcaggaaacatat

SEQ ID NO: 58 - DNA, 205 bp, Homo sapiens; identical to SEQ ID NO: 57

SEQ ID NO: 59 - DNA, 126 bp, Homo sapiens
tcccaatgaagaaactttcacatgtcttactctctgtcctagtcccagagcctgtaaaggtgaacccactgggactggctgggggagaagaggaagatttgttccagaaggaactgtctgagggat

SEQ ID NO: 60 - DNA, 67 bp, Homo sapiens
ttcctatgatgaggaccttttcacagacctgtactgagctccgtgaggataaataactctgaggaga

SEQ ID NO: 61 - DNA, 75 bp, Homo sapiens
ctacggggatgattttacgaactgaactctctctttctgatggattagtggagaaaacagaaaattctgagtagc

SEQ ID NO: 62 - DNA, 72 bp, Homo sapiens
actccatgatgaacacaaaatgacaagcatatggctgaactttcaagtgatgtcatcttactactgagaagt

SEQ ID NO: 63 - DNA, 75 bp, Homo sapiens
gtcagatgatttgaattgataagctgatgttctgtgaggtacaaaagttaatagcatgttagagttctgatggca

SEQ ID NO: 64 - DNA, 65 bp, Homo sapiens
tttctatgatgaatcaaactagctcactatgaccgacagtgaaaatacatgaacacctgagaaac

SEQ ID NO: 65 - DNA, 217 bp, Homo sapiens
aagactatactttcagggatcatttctatagtgtgttactagagaagtttctctgaacgtgtagagcaccgaaaaccccgaggaagagaggtagcgttttctcctgagcgtgaagccggctttctggcgttgcttggctgcaactgccgtcagccattgatgatcgttcttctctccgtattggggagtgagagggagagaacgcggtctgagtggt

SEQ ID NO: 66 - DNA, 70 bp, Homo sapiens
gtttgtgatgacttacatggaatctcgttcggctgatgacttgctgttgagactctgaaatctgattttc

SEQ ID NO: 67 - DNA, 71 bp, Homo sapiens
ctcaccagtgatgagttgaataccgccccagtctgatcaatgtgtgactgaaaggtattttctgagctgtg

SEQ ID NO: 68 - DNA, 77 bp, Homo sapiens
gtcagtgatgagcaacattcaccatctttcgtttgagtctcacggccatgagatcaaccccatgcaccgctctgaga

SEQ ID NO: 69 - DNA, 83 bp, Homo sapiens
ggccggtgatgagaacttctcccactcacattcgagtttcccgaccatgagatgactccacatgcactaccatctgaggccac

SEQ ID NO: 70 - DNA, 66 bp, Homo sapiens
cgtccatgatgttccgcaactacctacattgtttgatcctcatgaaagcagcactggctgagacgc

SEQ ID NO: 71 - DNA, 72 bp, Homo sapiens
ttgcaatgatgtgaatctctcactgaattcaaccttgaagtgcgaatccatgagctttttaaccctgagcaa

SEQ ID NO: 72 - DNA, 71 bp, Homo sapiens
ttctcgtgatgaaaactctgtccagttctgctactgaagggagagagatgagagccttttaggctgaggaa

SEQ ID NO: 73 - DNA, 69 bp, Homo sapiens
tctcagtgatgaaaactttgtccagttctgctactgacagtaagtgaagataaagtgtgtctgaggaga

SEQ ID NO: 74 - DNA, 70 bp, Homo sapiens
tgggaagtgatgacacctgtgactgttgatgtggaactgatttatcgcgtattcgtactggctgatcctg

SEQ ID NO: 75 - DNA, 62 bp, Homo sapiens
cacagatgatgaacttattgacgggcggacagaaactgtgtgctgattgtcacgttctgatt

SEQ ID NO: 76 - DNA, 64 bp, Homo sapiens
agtgatgatgaccccaggtaactcttgagtgtgtcgctgatgccatcaccgcagcgctctgacc

SEQ ID NO: 77 - DNA, 71 bp, Homo sapiens
tgctctgatgaaatcactaataggaagtgccgtcagaagcgataactgacgaagactactcctgtctgatt

SEQ ID NO: 78 - DNA, 71 bp, Homo sapiens; identical to SEQ ID NO: 77

SEQ ID NO: 79 - DNA, 48 bp, Homo sapiens
ctgatgatacttgtaataggaagtgccgtcagaagcgataactgacga

SEQ ID NO: 80 - DNA, 48 bp, Homo sapiens; identical to SEQ ID NO: 79

SEQ ID NO: 81 - DNA, 70 bp, Homo sapiens
gttgcatgatgaataaaatcaaatcaccatctttcggctgagttcgtgatggatttgcttttttctgatt

SEQ ID NO: 82 - DNA, 64 bp, Homo sapiens
gggaatgatgatttcacagactagagtctccgatgctggtcatgatgtcaaaactaagttctga

SEQ ID NO: 83 - DNA, 74 bp, Homo sapiens
gtgtatgatgacaactcggtaatgctgcatactcccgagtgcgcggtggggaagccaaccttggagagctgagc

SEQ ID NO: 84 - DNA, 74 bp, Homo sapiens; identical to SEQ ID NO: 83

SEQ ID NO: 85 - DNA, 71 bp, Homo sapiens
ccacaatgatggcaatatttttcgtcaacagcagttcacctagtgagtgttgagactctgggtctgagtga

SEQ ID NO: 86 - DNA, 71 bp, Homo sapiens; identical to SEQ ID NO: 85

SEQ ID NO: 87 - DNA, 72 bp, Homo sapiens
tggaggtgatgaactgtctgagcctgaccttgtagaatggaggcaaaaaaactgatttaatgagcctgatcc

SEQ ID NO: 88 - DNA, 75 bp, Homo sapiens
tattcctcactgatgagtacgttctgactttcgttcttctgagtttgctgaagccagatgcaatttctgagaagg

SEQ ID NO: 89 - DNA, 134 bp, Homo sapiens
actctctcggctctgcatagttgcacttggcttcacccgtgtgactttcgtaacggggagagagagaaaagatctcctcaggacctcggatgggccttactgtggcctctctttccttgaggggtgcaacaggc

SEQ ID NO: 90 - DNA, 137 bp, Homo sapiens
atccaaggtgattccctctccaaggggacatcagtgcctctcaggaaagtagcagcttggaatagaatctggcatgcctaaggcctttggggaactgggatgcttatttcctctgccttccttggctgcccacatgg

SEQ ID NO: 91 - DNA, 137 bp, Homo sapiens; identical to SEQ ID NO: 90

SEQ ID NO: 92 - DNA, 133 bp, Homo sapiens
attgcacctaaacccaagaatcactgtttcttatagcggtggtttaaacagaggtgcaaacagcaagcggatcttgtcgcctttggggggctgtggccgtgcccctcaaagtgaatttggaggttccacaact

SEQ ID NO: 93 - DNA, 133 bp, Homo sapiens; identical to SEQ ID NO: 92

SEQ ID NO: 94 - DNA, 138 bp, Homo sapiens
ctcctgcatccgaaagtgatcataggctgcctgtgcctaggtcattgatagtgcagggagaggaacgctgaaagaggttgtccccgtgtttggagggtccaccctatcccatccgaaccctgaagctttcacacaact

SEQ ID NO: 95 - DNA, 138 bp, Homo sapiens
tccctgtattcgaaagtgatcgtgggctgcctttgccctggtcattgatagtgcagggagaggaatcaatgaaagcgcttccccgtgtttgaagggtccactcctatcccttccaaactctggagctttcgtacatgc

SEQ ID NO: 96 - DNA, 138 bp, Homo sapiens
cacctgtattcgaaagtgatcgtgggctgcctgtgccctggtcattgatagtgcagggaaagaaatcgcggaaagtgcttccccgtgtttggagggtccgctcctgtccctttcaaactctggagctttctcacacct

SEQ ID NO: 97 - DNA, 138 bp, Homo sapiens; identical to SEQ ID NO: 96

SEQ ID NO: 98 - DNA, 72 bp, Homo sapiens
ctgcctctgatgaagcctgtgttggtagggacatctgagagtaatgatgaatgccaaccgctctgatggtgg

SEQ ID NO: 99 - DNA, 60 bp, Homo sapiens
agcctgtgatgctttaagagtagtggacagaagggatttctgaaattctattctgaggct

SEQ ID NO: 100 - DNA, 65 bp, Homo sapiens
gtgtaatgatgttgatcaaatgtctgacctgaaatgagcatgtagacaaaggtaacactgaagaa

SEQ ID NO: 101 - DNA, 65 bp, Homo sapiens; identical to SEQ ID NO: 100

SEQ ID NO: 102 - DNA, 93 bp, Homo sapiens
gctgttcagtgatgaggcctggaatgtgcgctgggcacagcgcccgagacagactgcggaaccgttccttgttgccttccttctgagaacagc

SEQ ID NO: 103 - DNA, 76 bp, Homo sapiens
gccaaatgatgtttatttgaaacaggagcacctcagtgcaaggacgactcttatctatcacccatgactgatggct

SEQ ID NO: 104 - DNA, 270 bp, Homo sapiens
caggctgatgagactaaggcgaatgcgactccgtgctctctggcccttggctccttgttgggggtggggactacagatgagatctgaaatcttagtggtagtacctgagccatgactccccactgtaaggccagatcaatagcattggtggccttgccttcatttctggtgctgcccctagttcctggcagcagcctgcagggaggcccacaggtggggtccacggtagggctgggcacaagccacctgagcgcaaccttggatctgaca

SEQ ID NO: 105 - DNA, 136 bp, Homo sapiens
atcgtcaggtgggataatccttacctgttcctcctccggagggcagattagaacatgatgattggagatgcatgaaacgtgattaacgtctctgcgtaatcaggacttgcaacaccctgattgctcctgtctgatt

SEQ ID NO: 106 - DNA, 63 bp, Homo sapiens
gcggtgatgaccccaacatgccatctgagtgtcggtgctgaaatccagaggctgtttctgagc

SEQ ID NO: 107 - RNA, 23 nt, Homo sapiens
ucaaaugcucagacuccuguggu

SEQ ID NO: 108 - RNA, 22 nt, Homo sapiens
cugcaauguaagcacuucuuac

SEQ ID NO: 109 - RNA, 23 nt, Homo sapiens
aaaagugcuuacagugcagguag

SEQ ID NO: 110 - RNA, 22 nt, Homo sapiens
ccgcacuguggguacuugcugc

SEQ ID NO: 111 - RNA, 21 nt, Homo sapiens
uaaagugcugacagugcagau

SEQ ID NO: 112 - RNA, 21 nt, Homo sapiens
ccgucgccgccacccgagccg

SEQ ID NO: 113 - RNA, 27 nt, Homo sapiens
cacuguaggugauggugagagugggca

SEQ ID NO: 114 - RNA, 21 nt, Homo sapiens
uggcagggaggcugggagggg

SEQ ID NO: 115 - RNA, 20 nt, Homo sapiens
ucacaccugccucgcccccc

SEQ ID NO: 116 - RNA, 19 nt, Homo sapiens
aauggauuuuuggagcagg

SEQ ID NO: 117 - RNA, 20 nt, Homo sapiens
uaaggcacgcggugaaugcc

SEQ ID NO: 118 - RNA, 24 nt, Homo sapiens
agccuggaagcuggagccugcagu

SEQ ID NO: 119 - RNA, 22 nt, Homo sapiens
acggguuaggcucuugggagcu

SEQ ID NO: 120 - RNA, 18 nt, Homo sapiens
cgggcguggugguggggg

SEQ ID NO: 121 - RNA, 22 nt, Homo sapiens
ucguaccgugaguaauaaugcg

SEQ ID NO: 122 - RNA, 22 nt, Homo sapiens
ucggauccgucugagcuuggcu

SEQ ID NO: 123 - RNA, 22 nt, Homo sapiens
cugaagcucagagggcucugau

SEQ ID NO: 124 - RNA, 22 nt, Homo sapiens
cuuggcaccuagcaagcacuca

SEQ ID NO: 125 - RNA, 17 nt, Homo sapiens
gugggggagaggcuguc

SEQ ID NO: 126 - RNA, 17 nt, Homo sapiens
ucgccuccuccucuccc

SEQ ID NO: 127 - RNA, 21 nt, Homo sapiens
ucacagugaaccggucucuuu

SEQ ID NO: 128 - RNA, 22 nt, Homo sapiens
uucuggaauucugugugaggga

SEQ ID NO: 129 - RNA, 22 nt, Homo sapiens
acucggcguggcgucggucgug

SEQ ID NO: 130 - RNA, 22 nt, Homo sapiens
ugugacugguugaccagagggg

SEQ ID NO: 131 - RNA, 21 nt, Homo sapiens
uaccacaggguagaaccacgg

SEQ ID NO: 132 - RNA, 23 nt, Homo sapiens
uguaguguuuccuacuuuaugga

SEQ ID NO: 133 - RNA, 21 nt, Homo sapiens
cauaaaguagaaagcacuacu

SEQ ID NO: 134 - RNA, 22 nt, Homo sapiens
ggugcagugcugcaucucuggu

SEQ ID NO: 135 - RNA, 21 nt, Homo sapiens
ugagaugaagcacuguagcuc

SEQ ID NO: 136 - RNA, 23 nt, Homo sapiens
guccaguuuucccaggaaucccu

SEQ ID NO: 137 - RNA, 22 nt, Homo sapiens
ucucccaacccuuguaccagug

SEQ ID NO: 138 - RNA, 22 nt, Homo sapiens
uugcauagucacaaaagugauc

SEQ ID NO: 139 - RNA, 22 nt, Homo sapiens
uagguuauccguguugccuucg

SEQ ID NO: 140 - RNA, 23 nt, Homo sapiens
uuaaugcuaaucgugauaggggu

SEQ ID NO: 141 - RNA, 22 nt, Homo sapiens
caggccauauugugcugccuca

SEQ ID NO: 142 - RNA, 22 nt, Homo sapiens
uagcagcacauaaugguuugug

SEQ ID NO: 143 - RNA, 22 nt, Homo sapiens
acugcagugaaggcacuuguag

SEQ ID NO: 144 - RNA, 23 nt, Homo sapiens
caaagugcuuacagugcagguag

SEQ ID NO: 145 - RNA, 22 nt, Homo sapiens
accacugaccguugacuguacc

SEQ ID NO: 146 - RNA, 22 nt, Homo sapiens
accaucgaccguugauuguacc

SEQ ID NO: 147 - RNA, 23 nt, Homo sapiens
aacauucaacgcugucggugagu

SEQ ID NO: 148 - RNA, 23 nt, Homo sapiens
aacauucauugcugucggugggu

SEQ ID NO: 149 - RNA, 22 nt, Homo sapiens
aaccaucgaccguugaguggac

SEQ ID NO: 150 - RNA, 22 nt, Homo sapiens
aacauucaaccugucggugagu

SEQ ID NO: 151 - RNA, 23 nt, Homo sapiens
aacauucauuguugucggugggu

SEQ ID NO: 152 - RNA, 22 nt, Homo sapiens
uggagagaaaggcaguuccuga

SEQ ID NO: 153 - RNA, 21 nt, Homo sapiens
caucccuugcaugguggaggg

SEQ ID NO: 154 - RNA, 23 nt, Homo sapiens
acugcccuaagugcuccuucugg

SEQ ID NO: 155 - RNA, 23 nt, Homo sapiens
uaaggugcaucuagugcagauag

SEQ ID NO: 156 - RNA, 23 nt, Homo sapiens
uaaggugcaucuagugcaguuag

SEQ ID NO: 157 - RNA, 22 nt, Homo sapiens
ccaauauuggcugugcugcucc

SEQ ID NO: 158 - RNA, 22 nt, Homo sapiens
gguccagaggggagauagguuc

SEQ ID NO: 159 - RNA, 23 nt, Homo sapiens
cccaguguucagacuaccuguuc

SEQ ID NO: 160 - RNA, 22 nt, Homo sapiens
acaguagucugcacauugguua

SEQ ID NO: 161 - RNA, 23 nt, Homo sapiens
ugugcaaaucuaugcaaaacuga

SEQ ID NO: 162 - RNA, 23 nt, Homo sapiens
ugugcaaauccaugcaaaacuga

SEQ ID NO: 163 - RNA, 23 nt, Homo sapiens
uaaagugcuuauagugcagguag

SEQ ID NO: 164 - RNA, 22 nt, Homo sapiens
acuguaguaugggcacuuccag

SEQ ID NO: 165 - RNA, 21 nt, Homo sapiens
caacaccagucgaugggcugu

SEQ ID NO: 166 - RNA, 22 nt, Homo sapiens
ugccugucuacacuugcugugc

SEQ ID NO: 167 - RNA, 22 nt, Homo sapiens
ugucaguuugucaaauacccca

SEQ ID NO: 168 - RNA, 21 nt, Homo sapiens
aggcggagacuugggcaauug

SEQ ID NO: 169 - RNA, 22 nt, Homo sapiens
cauugcacuugucucggucuga

SEQ ID NO: 170 - RNA, 21 nt, Homo sapiens
uucacaguggcuaaguuccgc

SEQ ID NO: 171 - RNA, 21 nt, Homo sapiens
auguaugugugcaugugcaug

SEQ ID NO: 172 - RNA, 22 nt, Homo sapiens
uaugugggaugguaaaccgcuu

SEQ ID NO: 173 - RNA, 22 nt, Homo sapiens
cugguuucacaugguggcuuag

SEQ ID NO: 174 - RNA, 22 nt, Homo sapiens
ugaccgauuucuccugguguuc

SEQ ID NO: 175 - RNA, 22 nt, Homo sapiens
uagcaccauuugaaaucgguua

SEQ ID NO: 176 - RNA, 20 nt, Homo sapiens
aaaagcuggguugagagggu

SEQ ID NO: 177 - RNA, 20 nt, Homo sapiens
acugccccaggugcugcugg

SEQ ID NO: 178 - RNA, 22 nt, Homo sapiens
cuagguauggucccagggaucc

SEQ ID NO: 179 - RNA, 21 nt, Homo sapiens
gaacggcuucauacaggaguu

SEQ ID NO: 180 - RNA, 23 nt, Homo sapiens
ucccuguccuccaggagcucacg

SEQ ID NO: 181 - RNA, 22 nt, Homo sapiens
cagugccucggcagugcagccc

SEQ ID NO: 182 - RNA, 23 nt, Homo sapiens
ucucacacagaaaucgcacccgu

SEQ ID NO: 183 - RNA, 21 nt, Homo sapiens
aggggugcuaucugugauuga

SEQ ID NO: 184 - RNA, 23 nt, Homo sapiens
ugucugcccgcaugccugccucu

SEQ ID NO: 185 - RNA, 22 nt, Homo sapiens
caaucacuaacuccacugccau

SEQ ID NO: 186 - RNA, 22 nt, Homo sapiens
aaucacuaaccacacggccagg

SEQ ID NO: 187 - RNA, 22 nt, Homo sapiens
uuaucagaaucuccagggguac

SEQ ID NO: 188 - RNA, 24 nt, Homo sapiens
aauccuuggaaccuaggugugagu

SEQ ID NO: 189 - RNA, 22 nt, Homo sapiens
cggguggaucacgaugcaauuu

SEQ ID NO: 190 - RNA, 22 nt, Homo sapiens
gccugcugggguggaaccuggu

SEQ ID NO: 191 - RNA, 21 nt, Homo sapiens
aacauagaggaaauuccacgu

SEQ ID NO: 192 - RNA, 22 nt, Homo sapiens
agagguugcccuuggugaauuc

SEQ ID NO: 193 - RNA, 21 nt, Homo sapiens
ugguagacuauggaacguagg

SEQ ID NO: 194 - RNA, 22 nt, Homo sapiens
uauacaagggcaagcucucugu

SEQ ID NO: 195 - RNA, 22 nt, Homo sapiens
gaaguuguucgugguggauucg

SEQ ID NO: 196 - RNA, 22 nt, Homo sapiens
gaauguugcucggugaaccccu

SEQ ID NO: 197 - RNA, 23 nt, Homo sapiens
agguuacccgagcaacuuugcau

SEQ ID NO: 198 - RNA, 21 nt, Homo sapiens
aauauaacacagauggccugu

SEQ ID NO: 199 - RNA, 21 nt, Homo sapiens
uaguagaccguauagcguacg

SEQ ID NO: 200 - RNA, 23 nt, Homo sapiens
agcucggucugaggccccucagu

SEQ ID NO: 201 - RNA, 23 nt, Homo sapiens
ugaggggcagagagcgagacuuu

SEQ ID NO: 202 - RNA, 21 nt, Homo sapiens
caaaacgugaggcgcugcuau

SEQ ID NO: 203 - RNA, 22 nt, Homo sapiens
cagcagcaauucauguuuugaa

SEQ ID NO: 204 - RNA, 22 nt, Homo sapiens
aucgggaaugucguguccgccc

SEQ ID NO: 205 - RNA, 21 nt, Homo sapiens
ugucuugcaggccgucaugca

SEQ ID NO: 206 - RNA, 23 nt, Homo sapiens
ucuuggaguaggucauugggugg

SEQ ID NO: 207 - RNA, 22 nt, Homo sapiens
aucaugaugggcuccucggugu

SEQ ID NO: 208 - RNA, 22 nt, Homo sapiens
gucauacacggcucuccucucu

SEQ ID NO: 209 - RNA, 22 nt, Homo sapiens
agaggcuggccgugaugaauuc

SEQ ID NO: 210 - RNA, 22 nt, Homo sapiens
aaucauacagggacauccaguu

SEQ ID NO: 211 - RNA, 22 nt, Homo sapiens
aaucguacagggucauccacuu

SEQ ID NO: 212 - RNA, 22 nt, Homo sapiens
gugacaucacauauacggcagc

SEQ ID NO: 213 - RNA, 22 nt, Homo sapiens
cuuaugcaagauucccuucuac

SEQ ID NO: 214 - RNA, 22 nt, Homo sapiens
ugaaggucuacugugugccagg

SEQ ID NO: 215 - RNA, 22 nt, Homo sapiens
ugaaacauacacgggaaaccuc

SEQ ID NO: 216 - RNA, 22 nt, Homo sapiens
aaacaaacauggugcacuucuu

SEQ ID NO: 217 - RNA, 22 nt, Homo sapiens
augcaccugggcaaggauucug

SEQ ID NO: 218 - RNA, 23 nt, Homo sapiens
uaauccuugcuaccugggugaga

SEQ ID NO: 219 - RNA, 22 nt, Homo sapiens
aaugcacccgggcaaggauucu

SEQ ID NO: 220 - RNA, 22 nt, Homo sapiens
aauccuuugucccugggugaga

SEQ ID NO: 221 - RNA, 22 nt, Homo sapiens
aaugcaccugggcaaggauuca

SEQ ID NO: 222 - RNA, 23 nt, Homo sapiens
uagcagcgggaacaguucugcag

SEQ ID NO: 223 - RNA, 22 nt, Homo sapiens
ccucccacacccaaggcuugca

SEQ ID NO: 224 - RNA, 22 nt, Homo sapiens
caugccuugaguguaggaccgu

SEQ ID NO: 225 - RNA, 22 nt, Homo sapiens
aaacauucgcggugcacuucuu

SEQ ID NO: 226 - RNA, 22 nt, Homo sapiens
gaaaucaagcgugggugagacc

SEQ ID NO: 227 - RNA, 22 nt, Homo sapiens
cacgcucaugcacacacccaca

SEQ ID NO: 228 - RNA, 23 nt, Homo sapiens
ugagugugugugugugagugugu

SEQ ID NO: 229 - RNA, 22 nt, Homo sapiens
uuaugguuugccugggacugag

SEQ ID NO: 230 - RNA, 25 nt, Homo sapiens
aggcaccagccaggcauugcucagc

SEQ ID NO: 231 - RNA, 21 nt, Homo sapiens
gaagugugccguggugugucu

SEQ ID NO: 232 - RNA, 23 nt, Homo sapiens
gcgaggaccccucggggucugac

SEQ ID NO: 233 - RNA, 20 nt, Homo sapiens
aggaauguuccuucuuugcc

SEQ ID NO: 234 - RNA, 22 nt, Homo sapiens
uccgagccugggucucccucuu

SEQ ID NO: 235 - RNA, 22 nt, Homo sapiens
guucucccaacguaagcccagc

SEQ ID NO: 236 - RNA, 21 nt, Homo sapiens
uggguuuacguugggagaacu

SEQ ID NO: 237 - RNA, 21 nt, Homo sapiens
agaccuggcccagaccucagc

SEQ ID NO: 238 - RNA, 21 nt, Homo sapiens
guggcugcacucacuuccuuc

SEQ ID NO: 239 - RNA, 21 nt, Homo sapiens
aauggcgccacuaggguugug

SEQ ID NO: 240 - RNA, 22 nt, Homo sapiens
uaugucugcugaccaucaccuu

SEQ ID NO: 241 - RNA, 22 nt, Homo sapiens
uacccauugcauaucggaguug

SEQ ID NO: 242 - RNA, 22 nt, Homo sapiens
gguggcccggccgugccugagg

SEQ ID NO: 243 - RNA, 20 nt, Homo sapiens
accaggaggcugaggccccu

SEQ ID NO: 244 - RNA, 23 nt, Homo sapiens
aggaagcccuggaggggcuggag

SEQ ID NO: 245 - RNA, 22 nt, Homo sapiens
uuugugaccugguccacuaacc

SEQ ID NO: 246 - RNA, 22 nt, Homo sapiens
acuccagccccacagccucagc

SEQ ID NO: 247 - RNA, 23 nt, Homo sapiens
ugcaccaugguugucugagcaug

SEQ ID NO: 248 - RNA, 28 nt, Homo sapiens
ucacaaugcugacacucaaacugcugac

SEQ ID NO: 249 - RNA, 26 nt, Homo sapiens
guuggaggaugaaaguacggagugau

SEQ ID NO: 250 - RNA, 23 nt, Homo sapiens
cugggaucuccggggucuugguu

SEQ ID NO: 251 - RNA, 22 nt, Homo sapiens
ugagaccucuggguucugagcu

SEQ ID NO: 252 - RNA, 22 nt, Homo sapiens
cugcccuggcccgagggaccga

SEQ ID NO: 253 - RNA, 21 nt, Homo sapiens
uccucuucucccuccucccag

SEQ ID NO: 254 - RNA, 20 nt, Homo sapiens
guagaggagauggcgcaggg

SEQ ID NO: 255 - RNA, 21 nt, Homo sapiens
gucagcggaggaaaagaaacu

SEQ ID NO: 256 - RNA, 23 nt, Homo sapiens
agguugggaucgguugcaaugcu

SEQ ID NO: 257 - RNA, 22 nt, Homo sapiens
ggguggggauuuguugcauuac

SEQ ID NO: 258 - RNA, 22 nt, Homo sapiens
uauugcacuugucccggccugu

SEQ ID NO: 259 - RNA, 22 nt, Homo sapiens
uauugcacucgucccggccucc

SEQ ID NO: 260 - RNA, 22 nt, Homo sapiens
acugcugagcuagcacuucccg

SEQ ID NO: 261 - RNA, 22 nt, Homo sapiens
acaguagagggaggaaucgcag

SEQ ID NO: 262 - RNA, 22 nt, Homo sapiens
ugcccuuaaaggugaacccagu

SEQ ID NO: 263 - RNA, 23 nt, Homo sapiens
caaagugcuguucgugcagguag